SEQUENCE LISTING (9 sequences)

SEQ ID NO: 1 - DNA, 10 bp, Artificial Sequence
  Synthetic tandem repeat unit
ccccctcccc

SEQ ID NO: 2 - DNA, 12 bp, Artificial Sequence
  Synthetic randomized barcode
  N (1)..(12): N = any nucleotide
  misc_feature (1)..(12): n is a, c, g, or t
nnnnnnnnnnnn

SEQ ID NO: 3 - DNA, 37 bp, Artificial Sequence
  Synthetic Custom Read1 Primer binding site
gcctgtccgcggaagcagtggtatcaacgcagagtac

SEQ ID NO: 4 - DNA, 16 bp, Artificial Sequence
  Synthetic insertion sequence
ttctctgttctttcat

SEQ ID NO: 5 - DNA, 16 bp, Artificial Sequence
  Synthetic insertion sequence
tctctgttctttcatg

SEQ ID NO: 6 - DNA, 15 bp, Artificial Sequence
  Synthetic deletion sequence
tttttcttcgcagga

SEQ ID NO: 7 - DNA, 15 bp, Artificial Sequence
  Synthetic deletion sequence
tcgcaggatttttct

SEQ ID NO: 8 - DNA, 19 bp, Artificial Sequence
  Synthetic forward primer for m.3243 region
cgccttcccccgtaaatga

SEQ ID NO: 9 - DNA, 19 bp, Artificial Sequence
  Synthetic reverse primer for m.3243 region
ggggcctttgcgtagttgt

(19) United States
(12) Patent Application Publication
(10) Pub. No.: US 2023/0235400 A1
(43) Pub. Date: Jul. 27, 2023

(54) METHODS OF DETECTING MITOCHONDRIAL DISEASES

(71) Applicants: The General Hospital Corporation, Boston, MA (US); Children's Medical Center Corporation, Boston, MA (US); President and Fellows of Harvard College, Cambridge, MA (US); The Broad Institute, Inc., Cambridge, MA (US); Massachusetts Institute of Technology, Cambridge, MA (US)

(72) Inventors: Vamsi Mootha, Boston, MA (US); Vijay Sankaran, Boston, MA (US); Caleb Lareau, Cambridge, MA (US); Melissa Walker, Boston, MA (US); Leif Ludwig, Cambridge, MA (US); Aviv Regev, Cambridge, MA (US)

(21) Appl. No.: 17/928,696

(22) PCT Filed: Jun. 4, 2021
(86) PCT No.: PCT/US2021/035951

§ 371 Date: Nov. 30, 2022

Related U.S. Application Data
(60) Provisional application No. 63/034,740, filed on Jun. 4, 2020.

(51) Int. Cl. C12Q 1/6883; G16B 30/00; G16B 20/00

(52) U.S. Cl. CPC C12Q 1/6883; G16B 30/00; G16B 20/00; C12Q 1/6869

(57) ABSTRACT

Described herein are methods of determining segregation dynamics of mitochondrial DNA herein. Also described herein are methods of diagnosing, prognosing, and/or monitoring a mitochondrial disease.

### CROSS-REFERENCE TO RELATED APPLICATIONS

This application claims the benefit of U.S. Provisional Application No. 63/034,740, filed Jun. 4, 2020. The entire contents of the above-identified applications are hereby fully incorporated herein by reference.

### STATEMENT REGARDING FEDERALLY SPONSORED RESEARCH

This invention was made with government support under Grant No. DK103794 awarded by the National Institutes of Health. The government has certain rights in the invention.

### SEQUENCE LISTING

This application contains a sequence listing filed in electronic form as an ASCII.txt file entitled BROD-5115WP_ST25.txt, created on Jun. 3, 2021 and having a size of 2,400 bytes (4 KB on disk). The content of the sequence listing is incorporated herein in its entirety.

### TECHNICAL FIELD

The subject matter disclosed herein is generally directed to identification and detection of diseases, such as mitochondrial diseases.

### BACKGROUND

Some of the most challenging mitochondrial disorders arise from mutations in mitochondrial DNA (mtDNA), a high copy number genome that is maternally inherited. These disorders present with marked clinical heterogeneity, in part because tissues generally contain a mixture of both wildtype and mutant mtDNA, a phenomenon called heteroplasmy. Given at least the limited understanding on the origin and nature of these diseases, there exists a need for improved treatments and preventions for these mitochondrial disorders.

Citation or identification of any document in this application is not an admission that such a document is available as prior art to the present invention.

### SUMMARY

Described in exemplary embodiments herein are methods of determining segregation dynamics of mitochondrial DNA (mtDNA) comprising: detecting mtDNA heteroplasmy and cell type and/or cell state in a cell or cell population, wherein detecting comprises, detecting, in a sample comprising the cell or cell population, a cell signature in the cell or cell population, and detecting mtDNA heteroplasmy in the cell or cell population, wherein the cell signature and/or mtDNA heteroplasmy indicates at least cell type and/or cell state.

In certain exemplary embodiments, the cell signature comprises a chromatin accessibility signature, a gene expression signature, a protein expression signature, an epigenetic state signature, a cell surface marker expression signature, a cell activity signature, a phenotypic profile, a cell landscape, or a combination thereof.

In certain exemplary embodiments, detecting the cell signature and/or detecting mtDNA heteroplasmy is/are determined by a sequencing method.

In certain exemplary embodiments, the sequencing method comprises single cell RNA sequencing and/or mitochondrial DNA single cell ATAC-seq (mtscATAC-seq).

In certain exemplary embodiments, detecting a cell signature comprises measuring a change in a distance in gene expression space between two or more cell states and/or measuring a change in a distance in accessible fragment space between two or more cell states.

In certain exemplary embodiments, gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

In certain exemplary embodiments, the distance in gene expression and/or accessible fragment space is measured by a Euclidean distance, Pearson coefficient, Spearman coefficient, or combination thereof.

In certain exemplary embodiments, detecting mtDNA heteroplasmy comprises detecting one or more mutations of the mtDNA.

In certain exemplary embodiments, at least one of the one or more mutations are pathogenic.

In certain exemplary embodiments, the at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and combinations thereof.

In certain exemplary embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof.

In certain exemplary embodiments, the cell or cell population comprises one or more circulating mononuclear cell(s) and wherein the cell signature comprises a circulating mononuclear cell signature.

In certain exemplary embodiments, the one or more cells comprise one or more peripheral blood mononuclear cells.

In certain exemplary embodiments, the one or more circulating mononuclear cells comprise lymphocyte(s), monocyte(s), dendritic cell(s) or a combination thereof.

In certain exemplary embodiments, the one or more circulating mononuclear cells comprise T cell(s), B cell(s), natural killer cell(s) or a combination thereof.

In certain exemplary embodiments, the sample is a bodily fluid, a bodily excretion, a bodily secretion, a tissue, a cell or cell population, or a combination thereof.

In certain exemplary embodiments, the sample is blood.

Also described in exemplary embodiments herein are methods of diagnosing, prognosing, and/or monitoring a mitochondrial disease comprising: detecting mitochondrial DNA (mtDNA) heteroplasmy and cell type and/or cell state in a cell or cell population, wherein detecting comprises detecting, in a sample comprising the cell or cell population, a cell signature in the cell or cell population, and detecting mtDNA heteroplasmy in the cell or cell population, wherein the cell signature and/or mtDNA heteroplasmy indicates at least cell type and/or cell state; and optionally repeating detecting mtDNA heteroplasmy and cell type and/or cell state one or more times over a period of time.

In certain exemplary embodiments, the cell signature comprises a chromatin accessibility signature, a gene expression signature, a protein expression signature, an epigenetic state signature, a cell surface marker expression signature, a cell activity signature, a phenotypic profile, a cell landscape, or a combination thereof.

In certain exemplary embodiments, detecting the signature and/or detecting mtDNA heteroplasmy is/are determined by a sequencing method.

In certain exemplary embodiments, the sequencing method comprises single cell RNA sequencing and/or mitochondrial DNA single cell ATAC-seq (mtscATAC-seq).

In certain exemplary embodiments, detecting a cell signature comprises measuring a change in a distance in gene expression or accessible fragment space between two or more cell states.

In certain exemplary embodiments, the gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

In certain exemplary embodiments, the distance in gene expression and/or accessible fragment space is measured by a Euclidean distance, Pearson coefficient, Spearman coefficient, or combination thereof.

In certain exemplary embodiments, detecting mtDNA heteroplasmy comprises detecting one or more mutations the mtDNA.

In certain exemplary embodiments, at least one of the one or more mutations are pathogenic.

In certain exemplary embodiments, the at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC (SEQ ID NO: 1)-tandem repeats at positions 305-314 and/or 956-965, deletion at positions from 8,469-13,447, 4,308-14,874, and/or 4,398-14,822, 961ins/delC, the mitochondrial common deletion (e.g., mtDNA 4,977 bp deletion), a mutation as set forth in any one or more of Tables 1-5, and combinations thereof.

In certain exemplary embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof.

In certain exemplary embodiments, the cell or cell population comprises one or more circulating mononuclear cell(s) and the cell signature comprises a circulating mononuclear cell signature.

In certain exemplary embodiments, the one or more circulating mononuclear cells comprise one or more peripheral blood mononuclear cells.

In certain exemplary embodiments, the one or more circulating mononuclear cells comprise lymphocyte(s), monocyte(s), dendritic cell(s) or a combination thereof.

In certain exemplary embodiments, the one or more circulating mononuclear cells comprise T cell(s), B cell(s), natural killer cell(s) or a combination thereof.

In certain exemplary embodiments, the sample is a bodily fluid, a bodily excretion, a bodily secretion, a tissue, a cell or cells, or a combination thereof.

In certain exemplary embodiments, the sample is blood.

In certain exemplary embodiments, the mitochondrial disease is a maternally inherited mitochondrial disease.

In certain exemplary embodiments, the mitochondrial disease is a heteroplasmic mitochondrial disease.

In certain exemplary embodiments, the mitochondrial disease is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease as set forth in any one or more of Tables 1-5, or a combination thereof.

Also described in exemplary embodiments herein are methods of treating and/or preventing a mitochondrial disease or a symptom thereof in a subject in need thereof comprising: diagnosing, prognosing, and/or monitoring a mitochondrial disease or a symptom thereof in the subject in need thereof as described elsewhere herein, wherein the sample is from the subject in need thereof, and; administering one or more agent(s) or formulations thereof to the subject in need thereof effective to treat and/or prevent the mitochondrial disease or symptom thereof.

Also described in exemplary embodiments herein are kits for diagnosing, prognosing, and/or monitoring a mitochondrial disease and/or determining segregation dynamics of mitochondrial DNA (mtDNA) comprising: a collection vessel configured to collect and/or contain a sample comprising a cell or cell population obtained from a body of a subject, wherein the sample is a bodily fluid, a bodily excretion, a bodily secretion, a tissue, a cell or cell population, or a combination thereof; instructions fixed in a tangible medium of expression that provides direction to collect the sample in the collection vessel and determine
(a) segregation dynamics of mtDNA,
(b) a diagnosis of a mitochondrial disease,
(c) a prognosis of a mitochondrial disease, or
(d) a combination thereof,
   and optionally monitor any one or more of (a)-(d) by a method comprising: detecting mitochondrial DNA (mtDNA) heteroplasmy and cell type and/or cell state in the cell or cell population, wherein detecting comprises detecting cell signature in the cell or cell population, and detecting mtDNA heteroplasmy in the cell or cell population, wherein the cell signature and/or mtDNA heteroplasmy indicates at least cell type and/or cell state; and optionally repeating detecting mtDNA heteroplasmy and cell type and/or cell state in the cell or cell population one or more times over a period of time.

In certain exemplary embodiments, the cell signature comprises a chromatin accessibility signature, gene expression signature, protein expression signature, epigenetic state signature, a cell surface marker expression signature, a cell activity signature, a phenotypic profile, a cell landscape, or a combination thereof.

In certain exemplary embodiments, detecting the cell signature and/or detecting mtDNA heteroplasmy is/are determined by a single cell sequencing method.

In certain exemplary embodiments, the single cell sequencing method comprises single cell RNA sequencing and/or mitochondrial DNA single cell ATAC-seq (mtscATAC-seq).

In certain exemplary embodiments, detecting a cell signature comprises measuring a change in a distance in gene expression space and/or accessible fragment space between two or more cell states.

In certain exemplary embodiments, the gene expression and/or accessible fragment space comprises 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

In certain exemplary embodiments, the distance in gene expression and/or accessible fragment space is measured by a Euclidean distance, Pearson coefficient, Spearman coefficient, or combination thereof.

In certain exemplary embodiments, detecting mtDNA heteroplasmy comprises detecting one or more mutations the mtDNA.

In certain exemplary embodiments, at least one of the one or more mutations are pathogenic.

In certain exemplary embodiments, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and combinations thereof.

In certain exemplary embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof.

In certain exemplary embodiments, cell or cell population comprises one or more circulating mononuclear cell(s) and the cell signature is a circulating mononuclear cell signature.

In certain exemplary embodiments, the one or more circulating mononuclear cells comprise one or more peripheral blood mononuclear cells.

In certain exemplary embodiments, the one or more circulating mononuclear cells comprise lymphocyte(s), monocyte(s), dendritic cell(s) or a combination thereof.

In certain exemplary embodiments, the one or more circulating mononuclear cells comprise T cell(s), B cell(s), natural killer cell(s) or a combination thereof.

In certain exemplary embodiments, sample is a bodily fluid, a bodily excretion, a bodily secretion, a tissue, a cell or cells, or a combination thereof.

In certain exemplary embodiments, the sample is blood.

In certain exemplary embodiments, the mitochondrial disease is a maternally inherited mitochondrial disease.

In certain exemplary embodiments, the mitochondrial disease is a heteroplasmic mitochondrial disease.

In certain exemplary embodiments, the mitochondrial disease is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease as set forth in any one or more of Tables 1-5, or a combination thereof.

In certain exemplary embodiments, the collection vessel comprises a reagent effective to prepare and/or preserve the sample.

In certain exemplary embodiments, the collection vessel comprises a reagent effective to prepare and/or preserve the sample for detecting the cell signature and/or mtDNA heteroplasmy.

In certain exemplary embodiments, the collection vessel is physically and/or chemically configured to preserve and/or prepare the sample for detecting the circulating mononuclear cell signature and/or mtDNA heteroplasmy.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—T cell-specific reduction in A3243G heteroplasmy in MELAS patients. UMAP depiction of patients P21, P9, P30 mtscATAC-seq data showing distribution of indicated major PBMC cell types (left-most panels). Histograms showing A3243G heteroplasmy fraction by indicated cell type for each of three patients with cell number N per population (HSC=hematopoietic stem cell, DC=dendritic cell, NK=natural killer) (center panels). Box plots are shown for per cell mtDNA coverage at m.3243 (second from the right) and for a proxy of mtDNA copy number (CN), i.e., the percentage of per cell reads aligning to mtDNA (right-most panel). Analyses exclude cells with a coverage at m.3243<20× or >1.5 interquartile ranges (IQRs) above the third quartile.

FIG. 2—Histogram of observed single A3243G heteroplasmy across all cell types in patient P21, restricting to cells with >100× mtDNA. 41 cells in the P21 dataset have >100× and <1.5 interquartile ranges above the third percentile coverage at m.3243.

FIG. 3—Cumulative distributions of A3243G heteroplasmy in MELAS patients. Cumulative distributions are stratified by cell type for the three indicated patient PBMCs profiled with mtscATAC-seq (DC=dendritic cell, NK=natural killer).

FIG. 4—Empirical determination of significance of the two sample Kolmogorov-Smirnov D statistic comparing T cells and all cells. The cell type label was permutated (i.e., T cell or not T cell, preserving the proportion of T cells observed in the respective patient). For each permuted dataset the two-sample K-S test statistic for the heteroplasmy CDF of “T cells” versus “all cells” under the permutation was computed. This procedure was repeated 100 times to generate a null distribution of K-S statistics, and compare to it the statistic obtained with the real data (Dobs) to the distribution of KS statistics obtained from the permuted data.

FIG. 5—Subdivision of T cell lineages reveals consistently lower percent A3243G heteroplasmy across all patients. Histograms show per cell A3243G heteroplasmy fraction in CD4+ and CD8+ T cells compared to other populations (DC=dendritic cell, NK=natural killer).

FIG. 6—Lack of correlation between A3243G heteroplasmy and mtDNA copy number in major PBMC cell types. For each patient P21, P9, and P30, per cell A3243G percent heteroplasmy (y axis) is plotted against the percentage of reads mapping to the mitochondrial genome (as a proxy of mtDNA copy number (CN) for each patient. Observed Spearman rank correlation coefficients (robs) are indicated in each panel with bootstrapped 95% confidence intervals shown in parentheses (DC=dendritic cell, NK=natural killer).

FIG. 7—Lack of correlation between A3243G heteroplasmy and mtDNA genome coverage and copy number in PBMCs. UMAPs for each indicated patient's PBMCs are presented colored by mitochondrial genomic coverage at position m.3243 (left column), percentage A3243G heteroplasmy (middle), and percentage of reads mapping to the mitochondrial genome (as a proxy of mtDNA copy number (CN), right).

FIG. 8—Patient clinical complete blood cell counts (where available). The mean value of all measured parameters is reported with standard deviation (SD) when multiple measurements were available. WBC=white blood cells, RBC=red blood cells, HGB=hemoglobin, HCT=hematocrit, PLT=platelets, MCV=mean corpuscular volume, MCH=mean corpuscular hemoglobin, MCHC=mean corpuscular hemoglobin concentration, RDW=red cell distribution width, MPV=mean platelet volume, NRBC=nucleated red blood cell, NEUTRO=neutrophils, LMYPHS=lymphocytes, MONOS=monocytes, EOS=eosinophils, BASOS=basophils, GRANULO, IMM=granulocytes, immature¬¬, k=thousand uL=microliter, g=gram, dL=deciliter, fl=femtoliter.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Heteroplasmic dynamics represent one of the most clinically and scientifically challenging and fascinating aspects of mtDNA disease. Bulk heteroplasmy measurements across tissue types and kindreds have failed to explain the origin, transmission, variability, and pathogenic mechanisms of pathologic mtDNA heteroplasmy. However, at least in some cases, longstanding observations have been made that at least in humans, bulk blood heteroplasmy is typically lowered compared to other tissues (see e.g., Grady J P et al. EMBO Mol Med 2018; De Laat et al. J Inherit Metab Dis 2012; and Maeda et al. JAMA Neurol 2016). Moreover, in some cases blood heteroplasmy has also been reported to decline with age (Grady J P et al. EMBO Mol Med 2018; De Laat et al. J Inherit Metab Dis 2012; Rahman et al. Am J Hum Genet 2002; and Pyle et al. J Med. Genet. 2007). However, the mechanisms underlying these observations remain unknown.

Single cell analysis of heteroplasmy holds the potential to be extremely powerful in studies of mtDNA heteroplasmy, but patient studies to date have been restricted to the study of one cell type at a time (primarily germline) at limited scale. Previous reports examined heteroplasmy in 82 oocytes (Brown et al. Random genetic drift determines the level of mutant mtDNA in human primary oocytes. 6tyAm J Hum Genet 2001) and 8 pancreatic beta cells (Lynn et al. Heteroplasmic ratio of the A3243G mitochondrial DNA mutation in single pancreatic beta cells. Dibetologia 2003) in a single A3243G patient each. Similarly, studies of T8993 heteroplasmy have reported restriction enzyme-based analysis in cells from single donors, including 87 oocytes (Blok et al. Skewed segregation of the mtDNA nt8993 (T→G) mutation in human oocytes. Am J Hum Genet 1997), 2 blastomeres (Steffann et al. Analysis of mtDNA variant segregation during early human embryonic development: A tool for successful NARP preimplantation diagnosis. J Med Genet 2006), and 30 lymphocytes (Gigarel et al. Single cell quantification of the 8993T>G NARP mitochondrial DNA mutation by fluorescent PCR. Mol Genet Metab 2005).

With at least these deficiencies in mind, embodiments disclosed herein provide methods of determining segregation dynamics of mitochondrial DNA (mtDNA). Determining and understanding the segregation dynamics is important to identifying and understanding mitochondrial diseases. Cells contain thousands of copies of the mitochondrial genome which are distributed within the tubular mitochondrial network that is spread across the cytosol of the cell. mtDNA replication occurs throughout the cell cycle ensuring that cells maintain a sufficient number of mtDNA copies. At replication termination the genomes must be resolved and segregated within the mitochondrial network. Defects in mtDNA replication and segregation result in various mitochondrial diseases, which ultimately result as a failure of cellular energy production. See e.g., Nicholls and Gustafsson. Trends Biochem. Sci. 2018. 43(11):869-881.

The methods of determining segregation dynamic of mtDNA can include detecting mtDNA heteroplasmy and cell type and/or cell state in a cell or cell population, wherein detecting includes detecting, in a sample comprising the cell or cell population, a cell signature in the cell or cell population, and detecting mtDNA heteroplasmy in the cell or cell population, where the cell signature and/or mtDNA heteroplasmy indicates at least cell type and/or cell state. Also provided herein are methods of diagnosing, prognosing, and/or monitoring a mitochondrial disease that can include detecting mitochondrial DNA (mtDNA) heteroplasmy and cell type and/or cell state in a cell or cell population, where detecting includes detecting, in a sample comprising the cell or cell population, a cell signature in the cell or cell population, and detecting mtDNA heteroplasmy in the cell or cell population, where the cell signature and/or mtDNA heteroplasmy indicates at least cell type and/or cell state; and optionally repeating detecting mtDNA heteroplasmy and cell type and/or cell state one or more times over a period of time. Also provided herein are methods of treating and/or preventing a mitochondrial disease or a symptom thereof in a subject in need thereof that can include diagnosing, prognosing, and/or monitoring a mitochondrial disease or a symptom thereof in the subject in need thereof as described herein, where the sample is from the subject in need thereof and administering one or more agent(s) or formulations thereof to the subject in need thereof effective to treat and/or prevent the mitochondrial disease or symptom thereof. Also provided herein are methods for diagnosing, prognosing, and/or monitoring a mitochondrial disease and/or determining segregation dynamics of mitochondrial DNA (mtDNA) including a collection vessel configured to collect and/or contain a sample comprising a cell or cell population obtained from a body of a subject, wherein the sample is a bodily fluid, a bodily excretion, a bodily secretion, a tissue, a cell or cell population, or a combination thereof; instructions fixed in a tangible medium of expression that provides direction to collect the sample in the collection vessel and determine a) segregation dynamics of mtDNA, b) a diagnosis of a mitochondrial disease, c) a prognosis of a mitochondrial disease, or d) a combination thereof, and optionally monitor any one or more of a)-d) by a method include detecting mitochondrial DNA (mtDNA) heteroplasmy and cell type and/or cell state in the cell or cell population, where detecting includes detecting cell signature in the cell or cell population, and detecting mtDNA heteroplasmy in the cell or cell population, where the cell signature and/or mtDNA heteroplasmy indicates at least cell type and/or cell state; and optionally repeating detecting mtDNA heteroplasmy and cell type and/or cell state in the cell or cell population one or more times over a period of time.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Determining Segregation Dynamics of Heteroplasmic DNA

Described herein are methods of determining segregation dynamics of mitochondrial DNA (mtDNA) that can include detecting mtDNA heteroplasmy and cell type and/or cell state in a cell or cell population, where detecting includes detecting, in a sample comprising the cell or cell population, a cell signature in the cell or cell population, and detecting mtDNA heteroplasmy in the cell or cell population, where the cell signature and/or mtDNA heteroplasmy indicates at least cell type and/or cell state.

As used herein, “cell state” is used to describe elements of a cell's identity. Cell state can be thought of as the characteristic profile or phenotype of a cell, which can be transient or permanent. Cell states can arise transiently during a process that can occur over a period of time. Temporal progression from one cell state to another can be unidirectional (e.g., during differentiation, or following an environmental stimulus) or can be in a state of vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These processes may occur transiently within a stable cell type (such as in a transient environmental response), or may lead to a new, distinct type (such as in differentiation). Wagner et al., 2016. Nat Biotechnol. 34(111): 1145-1160. As used herein, “cell type” refers to the more permanent aspects (e.g., a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

Described herein are methods to detect distinct cells and cell populations that can be identified by the unique signature of the specific cells and/or mtDNA heteroplasmy present. As used herein a signature can encompass any epigenetic profile or status, chromatin state or status, gene or genes, or protein or proteins, phenotypic profile, activity or cell landscape in a population whose occurrence is associated with a specific cell type, subtype, or cell state of a specific cell type or subtype within a population of cells. Increased or decreased expression or activity or prevalence may be compared between different cells in order to characterize or identify for instance specific cell (sub)populations. A gene signature as used herein, may thus refer to any set of up- and down-regulated genes between different cells or cell (sub)populations derived from a gene-expression profile. For example, a gene signature can be composed of a list of genes differentially expressed in a distinction of interest. It is to be understood that also when referring to proteins (e.g., differentially expressed proteins), such may fall within the definition of “gene” signature.

The signatures as defined herein (being it a gene signature, protein signature or other signature described herein) can be used to indicate the presence of a cell type, a subtype of the cell type, the state of the microenvironment of a population of cells, a particular cell type population or subpopulation, disease state, and/or the overall status of the entire cell (sub)population. Furthermore, the signature may be indicative of cells within a population of cells in vivo. The signature may also be used to suggest for instance particular therapies, or to follow up treatment, or to suggest ways to modulate cells, tissues, organs, and/or organ systems.

The presence of subtypes or cell states may be determined by subtype specific or cell state specific signatures. The presence of these specific cell (sub)types or cell states may be determined by applying the signature genes to bulk sequencing data in a sample. Not being bound by a theory, a combination of cell subtypes having a particular signature can indicate an outcome. Not being bound by a theory, the signatures can be used to deconvolute the network of cells present in a particular pathological condition. Not being bound by a theory, the presence of specific cells and cell subtypes are indicative of a particular response to treatment, such as including increased or decreased susceptibility to treatment. The cell signature can indicate the presence of one particular cell type. In one embodiment, the cell signatures are used to detect multiple cell states or hierarchies that occur in subpopulations of cells that are linked to particular pathological condition (e.g., a mitochondrial disease), or linked to a particular outcome or progression of the disease, or linked to a particular response to treatment of the disease.

In some embodiments, the cell signature is a chromatin accessibility signature, a gene expression signature, a protein expression signature, an epigenetic state signature, a cell surface marker expression signature, a cell activity signature, a phenotypic profile, a cell landscape, or a combination thereof. In some embodiments, the cell signature is uniquely associated with cell types, subtypes, states, including normal and dysfunctional and/or diseased states, and is analyzed and used to uniquely identify a particular cell state (e.g., normal or dysfunctional) and/or cell type. In some embodiments, the cell signature is associated with a disease, such as a mitochondrial disease, or a symptom thereof, including but not limited to those caused by or involving mtDNA heteroplasmy. In some embodiments, the cell signature is associated with mtDNA heteroplasmy and/or degree thereof. In some embodiments, the cell signature along with mtDNA heteroplasmy is associated with a disease, such as a mitochondrial disease or a symptom thereof. The cell signatures can be used to evaluate presence of, stage, or other characteristic or resulting phenotype of mtDNA heteroplasmy, disease resulting therefrom, and/or a symptom thereof, such as to specifically evaluate and target a disease or dysfunctional state while leaving normal (non-diseased) states intact. In some embodiments, the cell signature is a circulating mononuclear cell signature.

The terms, “cell landscape”, “cellular landscape”, are used interchangeably herein to refer to the possible and/or actual profile of cell states and/or cell types present within a defined cell population, such as a tissue, sample, organ, system, and the like. For example, in some embodiments the stromal cell landscape can include cells in various states. Remodeling of the cellular landscape can occur by various methods, such that the relative number of each cell state and/or cell type within the defined cell population is changed. This can occur, for example, by adding and/or removing cells of a specific cell state and/or type from the defined cell population and/or modulating the signatures of one or more cells such that they shift cell state and thus alter the relative number of each cell in the defined population. In some embodiments, diseases can result in remodeling a cell landscape such that the cell landscape is pathogenic or supportive of a disease state and/or disease development. In some embodiments, a diseased cell landscape is remodeled such that it is no longer diseased but is like or more like a homeostatic and/or beneficial cell landscape. Remodeling can occur by any suitable process or technique. In some embodiments, remodeling occurs as the result of exposure/administration of a compound (e.g., therapeutic agent) or system (e.g., a gene editing system) to a subject, diseased cell, diseased mitochondria, and/or diseased polynucleotides.

As used herein, “chromatin accessibility” refers to the degree to which nuclear macromolecules are able to physically contact chromatinized nuclear DNA and can be determined by the occupancy and topological organization of nucleosomes as well as other chromatin-binding factors that occlude access to DNA. Chromatin accessibility can be measured by any suitable method, including, but not limited to, sequencing methods such as ChIP-seq, ATAC-seq, DNase-seq, FAIRE-seq, MNase-seq, and others (see e.g., Tsompana and Buck. 2014. Epigenetics & Chromatin. 7(33) and Klemm S L et al. 2019. Nat. Rev. 20(4):207-220). As used herein “chromatin accessibility signature” is unique chromatin accessibility that can be used alone or in combination with other signatures to specifically identify a particular cell type, subtype, and/or state of a cell or cells within a cell population.

As used herein, “epigenetic state signature” refers to the unique epigenetic state that can be used alone or in combination with other signatures to specifically identify a particular cell type, subtype, and/or state of a cell or cells within a cell population.

As used herein, “cell activity state signature” refers to the unique cell activity or activities that can be used alone or in combination with other signatures to specifically identify a particular cell type, subtype, and/or state of a cell or cells within a cell population. As used herein, “cell activity” refers to any measurable or observable activity or functionality of a cell.

As used herein, “phenotypic profile” refers to a set of phenotypes that are characteristic of a cell type, subtype, and/or cell state and can be used alone or in combination with one or more signatures or other profiles to specifically identify a particular cell type, subtype, and/or state of a cell or cells within a cell population.

The signature according to certain embodiments of the present invention may comprise or consist of one or more genes and/or proteins, such as for instance 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of two or more genes and/or proteins, such as for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of three or more genes and/or proteins, such as for instance 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of four or more genes and/or proteins, such as for instance 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of five or more genes and/or proteins, such as for instance 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of six or more genes and/or proteins, such as for instance 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of seven or more genes and/or proteins, such as for instance 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of eight or more genes and/or proteins, such as for instance 8, 9, 10 or more. In certain embodiments, the signature may comprise or consist of nine or more genes and/or proteins, such as for instance 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more. In certain embodiments, the signature may comprise or consist of ten or more genes and/or proteins, such as for instance 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, to/or 50 or more.

In some embodiments, the cell signature can include one or more genes and/or proteins that are differentially expressed between different signatures. It is to be understood that “differentially expressed” genes/proteins include genes/proteins which are up- or down-regulated as well as genes/proteins which are turned on or off. When referring to up-or down-regulation, in certain embodiments, such up- or downregulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells. The upregulation and/or downregulation of gene or gene product, including the amount, may be included as part of the gene signature or expression profile.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥00% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

As discussed herein, differentially expressed genes/proteins may be differentially expressed on a single cell level, or may be differentially expressed on a cell population level. Preferably, the differentially expressed genes/proteins as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein of the signature, such as for instance at least to, at least three, at least four, at least five, at least six, or all genes/proteins of the signature.

Signatures may be functionally validated as being uniquely associated with a particular phenotype at the cell organelle, cell, tissue, organ, organ system, and/or organism level. Induction or suppression of a particular signature can consequentially be associated with or causally drive a particular cell organelle, cell, tissue, organ, organ system, and/or organism phenotype.

The signatures described herein can be detected, measured, or otherwise evaluated by a suitable analysis technique. In some embodiments, such techniques include a polynucleotide sequencing method, polypeptide sequencing methods, immunodetection techniques, polynucleotide hybridization-based techniques, cell activity assays, and combinations thereof. In some embodiments, the cell signature(s) can be detected by immunofluorescence, mass cytometry (CyTOF), FACS, drop-seq, RNA-seq, single-cell sequencing techniques (e.g. scRNA-seq) single cell qPCR, MERFISH (multiplex (in situ) RNA FISH), microarray and/or by in situ hybridization. Other methods including, but not limited to, absorbance assays and colorimetric assays are known in the art and can be used herein. In some embodiments, measuring expression of signature genes can include measuring protein expression levels. Protein expression levels can be measured, for example, by performing a Western blot, an ELISA or binding to an antibody array. In another aspect, measuring expression of said genes comprises measuring RNA expression levels. RNA expression levels may be measured by performing RT-PCR, Northern blot, an array hybridization, or RNA sequencing methods, Methods of detecting a signature, such as a gene signature, are described in greater detail elsewhere herein. Further details of some suitable sequencing methods are described in greater detail elsewhere herein.

In some embodiments, the signature can be obtained from cells using a single cell sequencing technique. In some embodiments the single cell sequencing technique can be or include scRNA-seq.

In some embodiments, signatures of the present invention can be discovered by analysis of cell signatures of single-cells within a population of cells from isolated samples (e.g., blood samples), thus allowing the discovery of previously unknown or unidentified cell subtypes or cell states that were previously invisible or unrecognized.

In some embodiments, identification of a specific cell type/subtype and/or state can include detecting a shift or change, such as a statistically significant shift or change, in the cell-state as indicated by a modulated (e.g., an increased or decreased distance) in the gene expression space between a first type/subtype and/or cell state to a second cell type/subtype and/or cell state. In some embodiments, the first or the second cell state is a dysfunctional or diseased cell state. In some embodiments, the dysfunction or diseased cell state is the result of bone marrow microenvironment remodeling by a cancer cell or cancer cell population. In certain embodiments, the distance is measured by a Euclidean distance, Pearson coefficient, Spearman coefficient, or combination thereof.

In some embodiments, detecting a cell signature can include or be measuring a change in a distance in gene expression space between two or more cell states and/or measuring a change in a distance in accessible fragment space between two or more cell states. In some embodiments, the gene expression and/or accessible fragment space comprises 1 to 1000 or more accessible genes and/or accessible fragments, such as 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 or more genes and/or accessible fragments. In some embodiments, the gene expression and/or accessible fragment space comprises 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

In certain embodiments, the shift in cell type and/or cell states that modulates the distance in expression (e.g., gene expression and/or protein expression) space between homeostatic cell-state and/or dysfunctional or diseased is a statistically significant shift in the gene expression distribution of the homeostatic and/or activated cell-state toward that of the dysfunctional or diseased cell state or away from the dysfunctional or diseased cell state. The statistically significant shift may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The statistical shift may include the overall transcriptional identity or the transcriptional identity of one or more genes, gene expression cassettes, or gene expression signatures of the dysfunctional or diseased cell state compared cell state (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the genes, gene expression cassettes, or gene expression signatures are statistically shifted in a gene expression distribution). A shift of 0% means that there is no difference to the homeostatic and/or dysfunctional cell state. A gene distribution may be the average or range of expression of particular genes, gene expression cassettes, or gene expression signatures in the homeostatic and/or dysfunctional or diseased cell-state (e.g., a plurality of a cell of interest from a subject may be sequenced and a distribution is determined for the expression of genes, gene expression cassettes, or gene expression signatures). In certain embodiments, the distribution is a count-based metric for the number of transcripts of each gene present in a cell. A statistical difference between the distributions indicates a shift. The one or more genes, gene expression cassettes, or gene expression signatures may be selected to compare transcriptional identity based on the one or more genes, gene expression cassettes, or gene expression signatures having the most variance as determined by methods of dimension reduction (e.g., tSNE analysis). In certain embodiments, comparing a gene expression distribution comprises comparing the initial cells with the lowest statistically significant shift as compared to the homeostatic and/or dysfunctional or diseased cell state (e.g., determining shifts when comparing only the dysfunctional or diseased cells with a shift of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% to the homeostatic cell state). In certain example embodiments, statistical shifts may be determined by defining a homeostatic, activated, and/or diseased/dysfunctional state score.

For example, a gene list of key genes enriched in a homeostatic/diseased model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the homeostatic score towards the dysfunctional or diseased score.

Other methods for assessing differences in the dysfunctional or diseased and cells may be employed. In certain embodiments, an assessment of differences in the dysfunctional or diseased and homeostatic cell epigenome and/or proteome may be used to further identify key differences in cell type and sub-types or cells. states. For example, isobaric mass tag labeling and liquid chromatography mass spectroscopy may be used to determine relative protein abundances in the ex vivo and in vivo systems. Description provided elsewhere herein further disclosure on leveraging proteome analysis within the context of the methods disclosed herein.

As discussed elsewhere herein, a collection of mRNA levels for a single cell can be called a gene expression profile (or expression signature) and is often represented mathematically by a vector in gene expression space. See e.g., Wagner et al., 2016. Nat. Biotechnol; 34(111): 1145-1160. This is a vector space that has a dimension corresponding to each gene, with the value of the ith coordinate of an expression profile vector representing the number of copies of mRNA for the ith gene. Note that real cells only occupy an integer lattice in gene expression space (because the number of copies of mRNA is an integer), but it is assumed herein that cells can move continuously through a real-valued G dimensional vector space.

As an individual cell changes the genes it expresses over time, it moves in gene expression space and describes a trajectory. As a population of cells develops and grows, a distribution on gene expression space evolves over time. When a single cell from such a population is measured with single cell RNA sequencing, a noisy estimate of the number of molecules of mRNA for each gene is obtained. The measured expression profile of this single cell is represented as a sample from a probability distribution on gene expression space. This sampling captures both (a) the randomness in the single cell RNA sequencing measurement process (due to sub-sampling reads, technical issues, etc.) and (b) the random selection of a cell from a population. This probability distribution is treated as nonparametric in the sense that it is not specified by any finite list of parameters.

A precise mathematical notion for a developmental process as a generalization of a stochastic process is provided below. A goal of the methods disclosed herein is to infer the ancestors and descendants of subpopulations evolving according to an unknown developmental, disease, and/or other physiological process and/or corresponding to a specific cell state at the beginning, end, or any point during the developmental process. While not bound by a particular theory, this may be possible over short time scales because it is reasonable to assume that cells don't change too much and therefore it can be inferred which cells go where. It will be appreciated that “developmental” when used in this context is not limited to the “growth/maturity” of an organism/cell, but rather refers to any characteristic that can change temporally and/or spatially such that the characteristic can be said to “develop” over time and/or space through a “developmental process”.

In certain example embodiments, the following definitions to define a precise notion of the developmental trajectory of an individual cell and its descendants are used. It is a continuous path in gene expression that bifurcates with every cell division.

Formally, consider a cell x(o)∈ [symbol] . Let k(t)+>0 specify the number of descendants at time t, where k(0)=1. A single cell developmental trajectory is a continuous function

$$x : [ 0 , T ) \to \underbrace{{ℝ}^{G} \times {ℝ}^{G} \times … \times {ℝ}^{G}}_{k ( t ) times} .$$

This means that x(t) is a k(t)-tuple of cells, each represented by a vector [symbol] :

x(t)=(x₁(t), . . . ,xₖ₍ₜ₎(t)).

Cells x₁(t), xₖ₍ₜ₎(t) as the descendants of x(o).

[symbol] and R^{G} are used interchangeably.

Note that the temporal dynamics of an individual cell cannot be directly measured because scRNA-Seq is a destructive measurement process: scRNA-Seq lyses cells so it is only possible to measure the expression profile of a cell at a single point in time. As a result, it is not possible to directly measure the descendants of that cell, and it is (usually) not possible to directly measure which cells share a common ancestor with ordinary scRNA-Seq. Therefore, the full trajectory of a specific cell is unobservable. However, one can learn something about the probable trajectories of individual cells by measuring snapshots from an evolving population.

Published methods typically represent the aggregate trajectory of a population of cells with a graph. While this recapitulates the branching path traveled by the descendants of an individual cell, it may over-simplify the stochastic nature of developmental processes. Individual cells have the potential to travel through different paths, but in reality any given cell travels one and only one such path. The methods disclosed herein help to describe this potential, which might not be a represented by a graph as a union of one-dimensional paths.

Instead, a developmental process is defined to be a time-varying distribution on gene expression space. The word distribution is used to refer to an object that assigns mass to regions of [symbol] . Note that a distinction is made between distribution and probability distribution, which necessarily has total mass 1. Distributions are formally defined as generalized functions (such as the delta function δ_{X}) that act on test functions. A used herein, a “distribution” is the same as a measure. One simple example of a distribution of cells is that a set of cells x₁, . . . , xₙ can be represented by the distribution

$$ℙ = {\sum }_{i = 1}^{n} {δ}_{{x}_{i}} .$$

Similarly, a set of single cell trajectories may be represented x₁(t), . . . , xₙ(t) with a distribution over trajectories. A developmental process [symbol] is a time-varying distribution on gene expression space. A developmental process generalizes the definition of stochastic process. A developmental process with total mass 1 for all time is a (continuous time) stochastic process, i.e. an ordered set of random variables with a particular dependence structure. Recall that a stochastic process is determined by its temporal dependence structure, i.e. the coupling between random variables at different time points. The coupling of a pair of random variables refers to the structure of their joint distribution. The notion of coupling for developmental processes is the same as for stochastic processes, except with general distributions replacing probability distributions.

A coupling of a pair of distributions P, Q on R^{G} is a distribution it on R^{G}×R^{G} with the property that it has P and Q as its two marginals. A coupling is also called a transport map.

As a distribution on the product space R^{G}×R^{G}, a transport map it assigns a number π(A, B) to any pair of sets A,B⊂R^{G}.

π(A,B)=ƒ_{xϵA}ƒ_{yϵB}π(x,y)dxdy.

When it is the coupling of a developmental process, this number π(A, B) represents the mass transported from A to B by the developmental or other process. This is the amount of mass coming from A and going to B. When a particular destination is note specified, the quantity π(A, ⋅) specifies the full distribution of mass coming from A. This action may be referred to as pushing A through the transport map π. More generally, we can also push a distribution μ forward through the transport map π via integration

μ [symbol] ƒπ(x,⋅)dμ(x).

The reverse operation is referred to as pulling a set B back through π. The resulting distribution π(⋅, B) encodes the mass ending up at B. Distributions μ can also be pulled back through π in a similar way:

μ [symbol] ƒπ(⋅,y)dμ(y).

This may also be referred as back-propagating the distribution μ (and to pushing μ forward as forward propagation).

Recall that a stochastic process is Markov if the future is independent of the past, given the present. Equivalently, it is fully specified by its couplings between pairs of time points. A general stochastic process can be specified by further higher order couplings. Markov developmental processes, which are defined in the same way:

A Markov developmental process Pₜ is a time-varying distribution on R^{G} that is completely specified by couplings between pairs of time points. It is an interesting question to what extent developmental processes are Markov. On gene expression space, they are likely not Markov because, for example, the history of gene expression can influence chromatin modifications, which may not themselves be reflected in the observed expression profile but could still influence the subsequent evolution of the process. However, it is possible that developmental processes could be considered Markov on some augmented space.

A definition of descendants and ancestors of subgroups of cells evolving according to a Markov developmental process is now provided. The earlier definition of descendants is extended as follows: Consider a set of cells S⊂R^{G}, which live at time t₁ are part of a population of cells evolving according to a Markov developmental process Pₜ. Let π denote the transport map for Pₜ from time t₁ to time t₂. The descendants of S at time t₂ are obtained by pushing S through the transport map π. Note that if a developmental process is not Markov, then the descendants of S are not well defined. The descendants would depend on the cells that gave rise to S, which we refer to as the ancestors of S.

Definition 6 (ancestors in a Markov developmental process). Consider a set of cells S ⊂R^{G}, which live at time t₂ and are part of a population of cells evolving according to a Markov developmental process Pₜ. Let π denote the transport map for Pₜ from time t₂ to time t₁. The ancestors of S at time t₁ are obtained by pushing S through the transport map π.

### Empirical Developmental Processes

In certain aspects, a goal of the embodiments disclosed herein is to track the evolution of a developmental process from a scRNA-Seq time course. Suppose we are given input data consisting of a sequence of sets of single cell expression profiles, collected at T different time slices of development. Mathematically, this time series of expression profiles is a sequence of sets S₁, . . . , S_{T}⊂R^{G} collected at times t₁, . . . , t_{T}∈R.

Developmental time series. A developmental time series is a sequence of samples from a developmental process Pₜ on R^{G}. This is a sequence of sets S₁, . . . , S_{N}⊂R^{G}. Each Sᵢ is a set of expression profiles in R^{G} drawn i.i.d from the probability distribution obtained by normalizing the distribution Pₜ_{ᵢ} to have total mass1. From this input data, we form an empirical version of the developmental process. Specifically, at each time point tᵢ we form the empirical probability distribution supported on the data x∈Sᵢ is formed. This is summarized in the following definition:

Empirical developmental process. An empirical developmental process \hat{P}ₜ is a time varying distribution constructed from a developmental time course S₁, . . . , S_{N}

$${\hat{ℙ}}_{{t}_{i}} = \frac{1}{\vert {S}_{i} \vert} {\sum }_{x ∈ {S}_{i}} {δ}_{x} .$$

the empirical developmental process is undefined for t∈/{t₁, . . . , t_{N}}.

The goal is to recover information about a true, unknown developmental process Pₜ from the empirical developmental process \hat{P}ₜ. The measurement process of single cell RNA-Seq destroys the coupling, and the observed empirical developmental process does not come with an informative coupling between successive time points. Over short time scales, it is reasonable to assume that cells do not change too much and therefore inferences regarding which cells go where and estimate the coupling.

This may be done with optimal transport: the transport map π that minimizes the total work required for redistributing \hat{P}ₜ_{ᵢ} to \hat{P}ₜ_{ᵢ₊₁}. is selected. One motivation for minimizing this objective, is a deep relationship between optimal transport and dynamical systems that provides a direct connection to Waddington's landscape: the optimal transport problem can formulated as a least-action advection of one distribution into another according to an unknown velocity field (see Theorem 1 in Section 6 below). At a high level, differentiation follows a velocity field on gene expression space, and the potential inducing this velocity field is in direct correspondence with Waddington's landscape¹.

Optimal Transport for scRNA-Seq Time Series

A process for how to compute probabilistic flows from a time series of single cell gene expression profiles by using optimal transport (S1) is provided. The embodiments disclosed herein show how to compute an optimal coupling of adjacent time points by solving a convex optimization problem.

Optimal transport defines a metric between probability distributions; it measures the total distance that mass must be transported to transform one distribution into another. For two measures P and Q on R^{G}, a transport plan is a measure on the product space R^{G}×R^{G} that has marginals P and Q. In probability theory, this is also called a coupling. Intuitively, a transport plan π can be interpreted as follows: if one picks a point mass at position x, then π(x, ⋅) gives the distribution over points where x might end up.

If c(x, y) denotes the cost² of transporting a unit mass from x to y, then the expected cost under a transport plan π is given by

ƒƒc(x,y)π(x,y)dxdy.

The optimal transport plan minimizes the expected cost subject to marginal constraints:

$${minimize}_{π} \int \int c ( x , y ) π ( x , y ) dxdy \\ subject to \int π ( x , \cdot ) dx = ℚ \\ \int π ( \cdot , y ) dy = ℙ .$$

Note that this is a linear program in the variable π because the objective and constraints are both linear in π. Note that the optimal objective value defines the transport distance between P and Q (it is also called the Earthmover's distance or Wasserstein distance). Unlike most other ways to compare distributions (such as KL-divergence or total variation), optimal transport takes the geometry of the underlying space into account. For example, the KL-Divergence is infinite for any two distributions with disjoint support, but the transport distance between two unit masses depends on their separation.

When the measures P and Q are supported on finite subsets of R^{G}, the transport plan is a matrix whose entries give transport probabilities and the linear program above is finite dimensional. In this context, empirical distributions are formed from the sets of samples S₁, . . . , S_{T}:

$${\hat{ℙ}}_{{t}_{i}} = \frac{1}{\vert {S}_{i} \vert} {\sum }_{x ∈ {S}_{i}} {δ}_{x} ,$$

were δ_{X} denotes the Dirac delta function centered at x∈R^{G}. These empirical distributions \hat{P}ₜ_{ᵢ} are definitely supported, and so it is possible solve the linear program[1] with P=\hat{P}ₜ_{ᵢ} and Q=\hat{P}ₜ_{ᵢ₊₁}.

However, the classical formulation [1] does not allow cells to grow (or die) during transportation (because it was designed to move piles of dirt and conserve mass). When the classical formulation is applied to a time series with two distinct subpopulations proliferating at different rates³, the transport map will artificially transport mass between the subpopulations to account for the relative proliferation. Therefore, we modify the classical formulation of optimal transport in equation [1] is modified to allow cells to grow at different rates.

Is it assumed that a cell's measured expression profile x determines its growth rate g(x). This is reasonable because many genes are involved in cell proliferation (e.g. cell cycle genes). It is further assumed g(x) is a known function (based on knowledge of gene expression) representing the exponential increase in mass per unit time, but also note that the growth rate can be allowed to be miss-specified by leveraging techniques from unbalanced transport (S2). In practice, g(x) is defined in terms of the expression levels of genes involved in cell proliferation.

Derivation of Transport with Growth

For any cell x∈Sᵢ₋₁, let r(x, y) be the fraction of x that transitions towards y. Then the amount of probability mass from x that ends up at y (after proliferation) is

r(x,y)g(x)^{Δ}^{ₜ},

where Δₜ=tᵢ₊₁−tᵢ. The total amount of mass that comes from x can be written two ways:

$${\sum }_{y ∈ {S}_{i + 1}} r ( x , y ) {g ( x )}^{{Δ}_{t}} \approx {g ( x )}^{{Δ}_{t}} d {\hat{ℙ}}_{{t}_{i}} ( x ) .$$

This gives us a first constraint. Similarly, there is also the constraint that the total mass observed at y is equal to the sum of masses coming from each x and ending up at y. In symbols,

$$d {\hat{ℙ}}_{{t}_{i + 1}} ( y ) {\sum }_{x ∈ {S}_{i}} {g ( x )}^{{Δ}_{t}} \approx {\sum }_{x ∈ {S}_{i}} r ( x , y ) {g ( x )}^{{Δ}_{t}} for each y ∈ {S}_{i + 1} .$$

The factor x∈Sᵢ^{g(x)}^{^{Δt}} on the left hand side accounts for the overall proliferation of all the cells from Sᵢ. Note that this factor is required so that the constraints are consistent: when one sums up both sides of the first constraint over x, this must equal the result of summing up both sides of the second constraint over y. Finally, for convenience these constraints are rewritten in terms of the optimization variable

π(x,y)=r(x,y)g(x)^{Δ}^{ₜ}.

Therefore, to compute the transport map between the empirical distributions of expression profiles observed at time tᵢ and tᵢ₊₁, the following linear program is set up:

$${minimize}_{π} {\sum }_{x ∈ {S}_{i}} {\sum }_{y ∈ {S}_{i + 1}} c ( x , y ) π ( x , y ) \\ subject to {\sum }_{x ∈ {S}_{i}} π ( x , y ) \approx d {\hat{ℙ}}_{{t}_{i + 1}} ( y ) {\sum }_{x ∈ {S}_{i}} {g ( x )}^{{Δ}_{t}} \\ {\sum }_{y ∈ {S}_{i + 1}} π ( x , y ) \approx d {\hat{ℙ}}_{{t}_{i}} ( x ) {g ( x )}^{{Δ}_{t}}$$

### Regularization and Algorithmic Considerations

Fast algorithms have been recently developed to solve an entropically regularized version of the transport linear program (S3). Entropic regularization means adding the entropy H(π)=E_{π} log π to the objective function, which penalizes deterministic transport plans (a purely deterministic transport plan would have only one nonzero entry in each row). Entropic regularization speeds up the computations because it makes the optimization problem strongly convex, and gradient ascent on the dual can be realized by successive diagonal matrix scalings (S3). These are very fast operations. This scaling algorithm has also been extended to work in the setting of unbalanced transport, where equality constraints are relaxed to bounds on KL-divergence (S2). This allows the growth rate function g(x) to be misspecified to some extent.

Both entropic regularization and unbalanced transport may be used. To compute the transport map between the empirical distributions of expression profiles observed at time tᵢ and tᵢ₊₁, the embodiments disclosed herein solve the following optimization problem:

$${minimize}_{π} {\sum }_{x ∈ {S}_{i}} {\sum }_{y ∈ {S}_{i + 1}} c ( x , y ) π ( x , y ) - ϵℋ ( π ) \\ subject to KL [ {\sum }_{x ∈ {S}_{i}} π ( x , y ) \vert \vert d {\hat{ℙ}}_{{t}_{i + 1}} ( y ) {\sum }_{x ∈ {S}_{i}} {g ( x )}^{{Δ}_{t}} ] \leq \frac{1}{{λ}_{1}} \\ KL [ {\sum }_{y ∈ {S}_{i + 1}} π ( x , y ) \vert \vert d {\hat{ℙ}}_{{t}_{i}} ( x ) {g ( x )}^{{Δ}_{t}} ] \leq \frac{1}{{λ}_{2}}$$

where ε, λ₁ and λ₂ are regularization parameters. This is a convex optimization problem in the matrix variable π∈R^{N}^{ᵢ}^{×N}^{₊₁}, where Nᵢ=|Sᵢ| is the number of cells sequenced at time tᵢ. It takes about 5 seconds to solve this unbalanced transport problem using the scaling algorithm of Chizat et al. 2016 (S2) on a standard laptop with Nᵢ≈5000. Note that the densities (on the discrete set Sᵢ) of the empirical distributions specified in equation [2] are simply d\hat{P}ₜ (x)=1. However, in principle one could use nonuniform empirical distributions (e.g. i Nᵢ if one wanted to include information about cell quality).

To summarize: given a sequence of expression profiles S₁, . . . , S_{T}, the optimization problem [5] for each successive pair of time points Sᵢ, Sᵢ₊₁ is solved. This gives us a sequence of transport maps.

To make this more precise, consider a single cell y∈Sᵢ. The column π(⋅, y) of the transport map π from tᵢ₋₁ to tᵢ describes the contributions to y of the cells in Sᵢ₋₁. This is the origin of y at the time point tᵢ₋₁. Similarly, the row r(y, ⋅) of the transition map from tᵢ to tᵢ₊₁ describes the probabilities y would transition to cells in Sᵢ₊₁. These are the fates of y, i.e. the descendants of y.

The origin of y further back in time may be computed via matrix multiplication: the contributions to y of cells in Sᵢ₋₂ are given by a column of the matrix

\tilde{π}_{[i-2,i]}=π_{[i-2,i-1]}π_{[i-1,i]}.

This matrix \hat{π}[i−2,i] represents the inferred transport from time point tᵢ₋₂ to tᵢ, and note it with a tilde to distinguish it from the maps computed directly from adjacent time points. Note that, in principle, the transport between any non-consecutive pairs of time points Sᵢ, Sⱼ, may be directly computed but it is not anticipated that the principle of optimal transport to be as reliable over long time gaps.

Finally, note that expression profiles can be interpolated between pairs of time points by averaging a cell's expression profile at time tᵢ with its fated expression profiles at time tᵢ₊₁

### Transport Maps Encode Regulatory Information

Transport maps can encode regulatory information, and provided herein are methods on how to set up a regression to fit a regulatory function to our sequence of transport maps. It is assumed that a cell's trajectory is cell-autonomous and, in fact, depends only on its own internal gene expression. This is wrong as it ignores paracrine signaling between cells, and we return to discuss models that include cell-cell communication at the end of this section. However, this assumption is powerful because it exposes the time-dependence of the stochastic process Pₜ as arising from pushing an initial measure through a differential equation:

x̅=f(x).

Here f is a vector field that prescribes the flow of a particle x. The biological motivation for estimating such a function f is that it encodes information about the regulatory networks that create the equations of motion in gene-expression space.

It is proposed to set up a regression to learn a regulatory function f that models the fate of a cell at time tᵢ₊₁ as a function of its expression profile at time tᵢ. For motivation that the transport maps might contain information about the underlying regulatory dynamics, we appeal to a classical theorem establishing a dynamical formulation of optimal transport.

Theorem 1 (Benamou and Brenier, 2001). The optimal objective value of the transport problem [1] is equal to the optimal objective value of the following optimization problem:

$${minimize}_{ρ , v} {\int }_{0}^{⊥} {\int }_{{ℝ}^{G}} {‖ v ( t , x ) ‖}^{2} ρ ( t , x ) dtdx \\ subject to ρ ( 0 , \cdot ) = ℙ , ρ ( 1 , \cdot ) = ℚ . \\ ∇ \cdot ( ρ v ) = \frac{\partial ρ}{\partial t}$$

In this theorem, v is a vector-valued velocity field that advects4 the distribution p from P to Q, and the objective value to be minimized is the kinetic energy of the flow (mass x squared velocity). Intuitively, the theorem shows that a transport map 7C can be seen as a point-to-point summary of a least-action continuous time flow, according to an unknown velocity field. While the optimization problem [8] can be reformulated as a convex optimization problem, and modified to allow for variable growth rates, it is inherently infinite dimensional and therefore difficult to solve numerically.

It is therefore proposed a tractable approach to learn a static regulatory function f from this sequence of transport maps. This approach involves sampling pairs of points using the couplings from optimal transport, and solving a regression to learn a regulatory function that predicts the fate of a cell at time tᵢ₊₁ as a function of its expression profile at time

### Regulatory Network Regression

For each pair of time points tᵢ,tᵢ₊₁, we consider the pair of random variables Xₜ,Xₜ jointly distributed according to r_{[t, t]}, (which we obtained from the i i+1 i i+1 transport map π_{[t}_{ᵢ}_{,t}_{_{i+1]}} by removing the effect of proliferation as in equation [3]). We set up the following optimization problem over regulatory functions f:

$${min}_{f ∈ ℱ} {𝔼}_{r} {‖ \frac{{X}_{{t}_{i}} - {X}_{{t}_{i + 1}}}{{Δ}_{t}} - f ( {X}_{{t}_{i}} ) ‖}^{2} .$$

Here F specifies a parametric function class to optimize over.

### Cell Non-Autonomous Processes

This section discusses an approach to cell-cell communication. Note that the gradient flow [8] only makes sense for cell autonomous processes. Otherwise, the rate of change in expression x is not just a function of a cell's own expression vector x(t), but also of other expression vectors from other cells. We can accommodate cell non-autonomous processes by allowing f to also depend on the full distribution Pₜ

$$\frac{dx}{dt} = f ( x , {ℙ}_{t} ) .$$

### Extensions to Continuous Time.

In this section it is discussed how this method could be improved by going beyond pairs of time points to track the continuous evolution of Pₜ. It is begun by pointing out a peculiar behavior of the method: whenever we have a time point with few sampled cells, our method is forced through an information bottleneck. As an extreme example—suppose there is a time point with only one cell. Everything would transition through that single cell, which is absurd! In this extreme case, we would be better off ignoring the time point. It is therefore proposed a smoothed approach that shares information between time slices and gracefully improves as data is added.

The continuous-time formulation is based on locally-weighted averaging, an elementary interpolation technique. Recall that given noisy function evaluations yᵢ≈f(xᵢ), one can interpolate f by averaging the yᵢ for all xᵢ close to a point of interest x:

$$f ( x ) \approx {\sum }_{i} {α}_{i} f ( {x}_{i} ) ,$$

where αᵢ are weights that give more influence to nearby points

In this setup, it is sought to interpolate a distribution-valued function Pₜ from the collections of i.i.d. samples S₁, . . . , S_{T}. We can interpolate a distribution-valued function by computing the barycenter (or centroid) of nearby time points with respect to the optimal transport metric. The transport barycenter of

$${minimize}_{ℚ} {\sum }_{i = 1}^{T} {α}_{i} {W}^{2} ( {ℙ}_{i} , ℚ ) ,$$

where W(P, Q) denotes the transport distance (or Wasserstein distance) between P and Q. The transport distance is defined by the optimal value of the transport problem [1]. The weights αᵢ can be chosen to interpolate about time point t by setting, for example,

$${minimize}_{ℚ} {\sum }_{i = 1}^{T} {α}_{i} {G}^{2} ( {\hat{ℙ}}_{{t}_{i}} , ℚ ) ,$$

where G(P, Q) denotes our modified transport distance from equation [5]. To solve this optimization problem, we can fix the support of Q to the samples observed at all time points ∪^{T}ᵢ₌₁Sᵢ. Then we can apply the scaling algorithm for unbalanced bary centers due to Chizat et al.

However, fixing the support of the barycenter ahead of time may not be completely satisfactory, and this motivates further research in the computation of transport bary centers: can we design an algorithm to solve for the barycenter Q without fixing the support in advance? Is there a dynamic formulation for bary centers analogous to the Brenier Benamou formula of Theorem 1, and can be leveraged to better learn gene regulatory networks?

Finally, this section is concluded with the observation that this continuous-time approach can provide a principled approach to sequential experimental design. Optimal time points can be identified for further data collection by examining the loss function (fit of barycenter) across time, and adding data where the fit is poor. Moreover, this continuous time approach can also be used to test the principle of optimal transport by withholding some time points and testing the quality of the interpolation against the held-out truth.

Such concepts, principles, and methods can be adapted and used with the present invention.

### Nucleic Acid Barcode, Barcode, and Unique Molecular Identifier (UMI)

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In some embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein in this context refers to a single mRNA or target nucleic acid to be sequenced. The UMI may also be used to determine the number of transcripts that gave rise to an amplified product, or in the case of target barcodes as described herein, the number of binding events. In some embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type (SEQ ID NO: 2), where each N is independently selected from any amino acid.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include incorporation of aminoallyl-labeled nucleotides, incorporation of sulfhydryl-labeled nucleotides, incorporation of allyl- or azide-containing nucleotides, and many other methods described in Bioconjugate Techniques (2nd Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

A desirable locus for DNA barcoding can be standardized so that large databases of sequences for that locus can be developed. Most of the taxa of interest have loci that are sequenceable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

Unique Molecular Identifiers are short (usually 4-10 bp) random barcodes added to transcripts during reverse-transcription. They enable sequencing reads to be assigned to individual transcript molecules and thus the removal of amplification noise and biases from RNA-seq data. Since the number of unique barcodes (4N, N-length of UMI) is much smaller than the total number of molecules per cell (˜106), each barcode will typically be assigned to multiple transcripts. Hence, to identify unique molecules both barcode and mapping location (transcript) must be used. UMI-sequencing typically consists of paired-end reads where one read from each pair captures the cell and UMI barcodes while the other read consists of exonic sequence from the transcript. UMI-sequencing typically consists of paired-end reads where one read from each pair captures the cell and UMI barcodes while the other read consists of exonic sequence from the transcript.

In some embodiments, the nucleic acids of the library are flanked by switching mechanism at 5′ end of RNA templates (SMART). SMART is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. (see, e.g., Zhu et al., 2001, Biotechniques. 30 (4): 892-7.

After processing the reads from a UMI experiment, the following conventions are often used: 1. The UMI is added to the read name of the other paired read. 2. Reads are sorted into separate files by cell barcode ° For extremely large, shallow datasets, a cell barcode may be added to the read name as well to reduce the number of files. A cell barcode indicates the cell from which mRNA is captured (e.g., Drop-Seq or Seq-Well).

### Sequencing Methods

As previously discussed in some embodiments, the cell signature is detected using a sequencing method. Many suitable sequencing methods and techniques are known in the art and are within the scope of this disclosure. Suitable sequencing methods for the cell signature include DNA sequencing techniques, RNA sequencing techniques, epigenetic status sequencing techniques (e.g., bisulfite sequencing), and polypeptide sequencing techniques.

Basic DNA sequencing methods suitable for use in some embodiments include those based on chemical degradation, primer extension/chain termination-based methods (e.g., Sanger sequencing), and shot-gun sequencing/analysis and others. High-throughput (both short-read and long-read) sequencing methods suitable for use in some embodiments include stepwise or “base-by-base” based methods, pyrosequencing, single molecule real-time sequencing, ion semiconductor sequencing, sequencing by synthesis, colony sequencing (used in Illumina's Hi-Seq sequencing machines), combinatorial probe anchor synthesis, sequencing by ligation, nanopore sequencing, genapsys sequencing, polony sequencing, nanoball sequencing, and massively parallel signature sequencing (MPSS), sequencing by hybridization and the like. Other suitable sequencing methods include, but are not limited to, microfluidic-based sequencing, microscopy based sequencing techniques (e.g., transmission electron microscopy DNA sequencing), RNAP (RNA polymerase)-based sequencing, and tunneling current-based sequencing. Suitable sequencing methods include single cell sequencing methods.

Sequencing Methods with Library Construction

In some embodiments, the sequencing method involves generation of a sequencing library. In some embodiments, the sequencing method includes constructing a sequencing library. The sequencing library can include a plurality of nucleic acids, where one or more of the nucleic acids can including a gene or polynucleotide of interest. In some embodiments, the library can be constructed such that each nucleic acid in the library can have a UMI and optionally a cell barcode. The libraries can be constructed preferably from any single cell sequencing technique, in some preferred embodiments, an mRNA sequencing protocol, in some embodiments, SMART-Seq. Any single cell sequencing protocol can be used, as described elsewhere herein, to construct the library. In some preferred embodiments, the protocol provides 3′ barcoded nucleic acids that are subjected to further steps in the method embodiments disclosed herein. Additional library construction methods are described elsewhere herein.

In some embodiments, an RNA library can be generated. In some embodiments, such as those using RNA-seq or single-cell RNA-seq an RNA library or single-cell RNA library can be generated. As used herein, RNA-seq methods refer to high-throughput single-cell RNA-sequencing protocols. RNA-seq includes, but is not limited to, Drop-seq, Seq-Well, InDrop and 1Cell Bio. RNA-seq methods also include, but are not limited to, smart-seq2, TruSeq, CEL-Seq, STRT, ChIRP-Seq, GRO-Seq, CLIP-Seq, Quartz-Seq, or any other similar method known in the art (see, e.g., “Sequencing Methods Review” Illumina® Technology, https://www.illumina. com/content/dam/illumina-marketing/documents/products/research reviews/sequencing-methods-review.pdf. See e.g., Wagner et al., 2016. Nat Biotechnol. 34(111): 1145-1160.

Generation of a sequencing library can include amplification of each nucleic acid in the library to create PCR products and can be utilize to derive polynucleotide information from a library. PCR-based and other amplification techniques can be utilized to amplify the library of nucleic acids. For PCR-based amplification techniques, primers can be utilized to drive amplification.

In some embodiments, any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In specific embodiments, the amplification reaction mixture may further comprise primers, capable of hybridizing to a target nucleic acid strand. The term “hybridization” refers to binding of an oligonucleotide primer to a region of the single-stranded nucleic acid template under the conditions in which primer binds only specifically to its complementary sequence on one of the template strands, not other regions in the template. The specificity of hybridization may be influenced by the length of the oligonucleotide primer, the temperature in which the hybridization reaction is performed, the ionic strength, and the pH. The term “primer” refers to a single stranded nucleic acid capable of binding to a single stranded region on a target nucleic acid to facilitate polymerase dependent replication of the target nucleic acid strand. Nucleic acid(s) that are “complementary” or “complement(s)” are those that are capable of base-pairing according to the standard Watson-Crick, Hoogsteen or reverse Hoogsteen binding complementarity rules.

“PCR” (polymerase chain reaction) refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

PCR encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture (see, e.g., Bernard et al., Anal. Biochem., 273:221-228, 1999 (two-color real-time PCR)). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Techniques for quantitative PCR are well-known to those of ordinary skill in the art, as exemplified in the following references: Freeman et al. (Biotechniques, 26:112-126, 1999; Becker-Andre et al. (Nucleic Acids Research, 17:9437-9447, 1989; Zimmerman et al. (Biotechniques, 21:268-279, 1996; Diviacco et al. (Gene, 122:3013-3020, 1992; Becker-Andre et al., (Nucleic Acids Research, 17:9437-9446, 1989); and the like.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually, primers are extended by a DNA polymerase. Primers usually have a length in the range of between 3 to 36 nucleotides, from 5 to 24 nucleotides, or from 14 to 36 nucleotides. In certain aspects, primers are universal primers or non-universal primers. Pairs of primers can flank a sequence of interest or a set of sequences of interest. Primers and probes can be degenerate in sequence. In certain aspects, primers bind adjacent to the target sequence, whether it is the sequence to be captured for analysis, or a tag that it to be copied.

In specific embodiments, the amplification reaction mixture may further comprise a first primer and optionally second primer. The first and second primer may comprise a portion that is complementary to a first portion of the target nucleic acid and a second primer comprising a portion that is complementary to a second portion of the target nucleic acid. The first and second primer may be referred to as a primer pair. In some embodiments, the first or second primer may comprise an RNA polymerase promoter.

In specific embodiments, the amplification reaction mixture may further comprise a polymerase. Subsequent to melting and hybridization with a primer, the nucleic acid is subjected to a polymerization step. A DNA polymerase is selected if the nucleic acid to be amplified is DNA. When the initial target is RNA, a reverse transcriptase may first be used to copy the RNA target into a cDNA molecule and the cDNA is then further amplified by a selected DNA polymerase. The DNA polymerase acts on the target nucleic acid to extend the primers hybridized to the nucleic acid templates in the presence of four dNTPs to form primer extension products complementary to the nucleotide sequence on the nucleic acid template.

In some embodiments, the library construction can include the step of enrichment. Nucleic acid enrichment reduces the complexity of a large nucleic acid sample, such as a genomic DNA sample, cDNA library or mRNA library, to facilitate further processing and genetic analysis. In certain example embodiments, the enrichment step is optional. In some embodiments, enrichment can be biotin-based or other purification-based enrichment of an amplified nucleic acid, such as a first PCR product. Specific enrichment example embodiments are described in greater detail elsewhere herein.

In some embodiments, the library construction can include a second amplification. In some embodiments, the second amplification can be a PCR-based amplification. Other amplification methods can also be used instead. Such methods are described elsewhere herein.

In some embodiments, a PCR-amplification based approach to derive genetic information from single-cell RNA-seq libraries. The method generally involves two PCR steps and size selection. Initially, a library is constructed wherein each sequence comprises a SMART sequence at the 5′ end and the 3′ end, a genetic region of interest at the 5′ end and a UMI and Cell BC at the 3′ end, e.g., 5′ SMART-genetic region of interest-UMI-Cell BC-SMART 3′.

A first PCR product is generated by amplifying sequences with a biotinylated 5′ primer comprising a binding site for a second PCR product and a sequence complementary to a specific gene of interest and a 3′ SMART primer complementary to the SMART sequence at the 3′ end of the nucleic acid to generate a first PCR product. The binding site for the second PCR product may be a partial Illumina sequencing primer binding site or an oligomer for sequencing kit, such as a NEBNext® oligos for Illumina® sequencing (see, e.g., https://www.neb.com/applications/library-preparation-for-next-generation-sequencing/illumina-library-preparation/products).

The 5′ primer comprising the binding site for the second PCR product to amplify the first PCR product may further comprise a sequence to bind a flow cell, a sequence allowing multiple sequencing libraries to be sequenced simultaneously and/or a sequence providing an additional primer binding site. The sequence to bind a flow cell may be a P7 sequence and the flow cell may be an Illumina® flowcell.

In another embodiment, the SMART primer complementary to the SMART sequence at the 3′ end of the nucleic acid to amplify the first PCR product may further comprise a sequence to allow fragments to bind a flowcell. The sequence to allow fragments to bind a flowcell may be a P5 sequence.

Regardless of the library construction method, submitted libraries may consist of a sequence of interest flanked on either side by adapter constructs. On each end, these adapter constructs may have flow cell binding sites, P5 and P7, which allow the library fragment to attach to the flow cell surface. The P5 and P7 regions of single-stranded library fragments anneal to their complementary oligos on the flowcell surface. The flow cell oligos act as primers and a strand complementary to the library fragment is synthesized. The original strand is washed away, leaving behind fragment copies that are covalently bonded to the flowcell surface in a mixture of orientations. 1,000 copies of each fragment are generated by bridge amplification, creating clusters. For simplification, the diagram shows only one copy (out of 1,000) in each cluster, and only two clusters (out of 30-50 million). The P5 region is cleaved, resulting in clusters containing only fragments which are attached by the P7 region. This ensures that all copies are sequenced in the same direction. The sequencing primer anneals to the P5 end of the fragment, and begins the sequencing by synthesis process. Index reads are only performed when a sample is barcoded. When Read 1 is finished, everything from Read 1 is removed and an index primer is added, which anneals at the P7 end of the fragment and sequences the barcode. Everything is stripped from the template, which forms clusters by bridge amplification as in Read 1. This leaves behind fragment copies that are covalently bonded to the flowcell surface in a mixture of orientations. This time, P7 is cut instead of P5, resulting in clusters containing only fragments which are attached by the P5 region. This ensures that all copies are sequences in the same direction (opposite Read 1). The sequencing primer anneals to the P7 region and sequences the other end of the template.

In another embodiment, the sequence allowing multiple sequencing libraries to be sequenced simultaneously may be an INDEX sequence. The INDEX allows multiple sequencing libraries to be sequenced simultaneously (and demultiplexed using Illumina's bcl2fastq command). See, e.g., https://support.illumina.com/downloads/illumina-customer-sequence-letter.html for exemplary INDEX sequences.

In another embodiment, the 5′ primer comprising the binding site for the second PCR product to amplify the first PCR product may further comprise a NEXTERA sequence. See, e.g., https://support.illumina.com/downloads/illumina-customer-sequence-letter.html and U.S. Pat. Nos. 5,965,443, and 6,437,109 and European Patent No. 0927258, for exemplary NEXTERA sequences.

In another embodiment, the sequence providing an additional primer binding site may be a custom read1 primer binding site (CR1P) for sequencing. CR1P is a Custom Read1 Primer binding site that is used for Drop-Seq and Seq-Well library sequencing. CR1P may comprise the sequence: GCCTGTCCGCGGAAGCAGTGGTATCAACGCAGAGTAC (SEQ ID NO: 3) (see e.g., Gierahn et al., Nature Methods 14, 395-398 (2017).

Biotin-NEXT-GENE-for: Biotinylation enables purification of the desired product following the first PCR reaction. NEXT creates a binding site for the second PCR product as well as a partial primer binding site for standard Illumina sequencing kits. NEXT may be any sequence that allows targeted enrichment and then select addition of sequencing handles. GENE is a sequence complementary to the WTA, designed to amplify a specific region of interest (usually an exon).

SMART-rev: The SMART sequence is used in Drop-seq and Seq-Well to generate WTA libraries. Because the polyT-unique molecular identifier-unique cellular barcode (polyT-UMI-CB) sequence is followed by the SMART sequence, and the template switching oligo (TSO) also contains the SMART sequence, WTA libraries have the SMART sequence as a PCR binding site on both the 5′ and the 3′ end.

P7-INDEX-NEXTERA: The P7 sequence allows fragments to bind the Illumina flowcell. The INDEX allows multiple sequencing libraries to be sequenced simultaneously (and demultiplexed using Illumina's bcl2fastq command). The NEXTERA sequence provides a primer binding site for Illumina's standard Read2 sequencing primer mix.

SMART-CR1P-P5: The SMART sequence is the same as in SMART-rev. CR1P is a Custom Read1 Primer binding site that is used for Drop-Seq and Seq-Well library sequencing. The P5 sequence allows fragments to bind the Illumina flowcell. Note that the primer design can be easily modified for compatibility with additional single-cell RNA-seq technologies (SMART) or sequencing technologies (NEXTERA, CR1P).

The method also provides for biotin enrichment of the first PCR product. Biotinylation of the primer to amplify the gene, region or mutation of interest from the library allows for the purification of the PCR product of interest. Because the libraries are flanked with SMART sequences on both ends, the vast majority of the first PCR product would be amplification of the entire library. Without the biotinylated primer, enrichment of the gene, region or mutation of interest would be insufficient to efficiently and confidently call genetic mutations. Biotin enrichment may be accomplished by streptavidin binding of the biotinylated first PCR product. The streptavidin bead kilobaseBINDER kit (Thermo Fisher Cat #60101) allows for isolation of large biotinylated DNA fragments.

Gene specific primers may be mixed for simultaneous detection of multiple mutations. Libraries may also be mixed for simultaneous detection of mutations in multiple samples. However, mixed primers sometimes may not detect multiple mutations in the same gene as only the shortest fragment will be detected.

The present method may be adapted to identify any gene, region or mutation of interest and to identify cells containing specific genes, regions or mutations, deletions, insertions, indels, or translocations of interest.

A gene or groups of genes of interest may be, for example, one or more genes that are part of or make up a homeostatic stromal cell gene expression signature, a dysfunctional stromal cell gene expression signature, or a combination thereof. The gene or groups of genes of interest may be, for example, a hematological disease-related gene of interest. Hematological diseases of interest are described in greater detail elsewhere herein.

In some embodiments, sequence adapters can be used. As used herein, sequence adapters or sequencing adapters or adapters include primers that may include additional sequences involved in for example, but not limited to, flowcell binding, cluster generation, library generation, sequencing primers, sequences for Seq-Well, and/or custom read sequencing primers. Universal primer recognition sequences

The present invention may encompass incorporation of SMART sequences into the library. Switching mechanism at 5′ end of RNA template (SMART) is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. (see, e.g., Zhu et al., 2001, Biotechniques. 30 (4): 892-7.

A pooled set of nucleic acids that are tagged refer to a plurality of nucleic acid molecules that results from incorporating an identifiable sequence tag into a pool of sample-tagged nucleic acids, by any of various methods. In some embodiments, the tag serves instead as a minimal sequence adapter for adding nucleic acids onto sample-tagged nucleic acids, rendering the pool compatible with a particular DNA sequencing platform or amplification strategy.

In some embodiments, a 3′ barcoded single cell RNA library can be generated. The 3′ barcoded single cell RNA library includes a plurality of nucleic acids, each nucleic acid including a gene of interest, a unique molecular identifier (UMI) and a cell barcode (cell BC). The cell barcode is located on the 3′ end of the transcript. As the single cell RNA library comprises a cell barcode on the 3′ end of the transcripts, at least a subset of the library from the 3′ barcoded single cell RNA library contains a transcript of interest at least 1 kb away from the 3′ end of the transcript. The 5′ side of transcripts are typically underrepresented in standard 3′ barcoded libraries.

In a preferred embodiment, each nucleic acid sequence is flanked by switching mechanism at 5′ end of RNA template (SMART) sequences at the 5′ end and 3′ end, that is, in this embodiment, an exemplary nucleic acid in the library would be 5′ SMART-genetic region of interest-UMI-Cell BC-SMART 3′.

Multiple technologies have been described that massively parallelize the generation of single cell RNA seq libraries that can be used in the present disclosure. As used herein, RNA-seq methods refer to high-throughput single-cell RNA-sequencing protocols. RNA-seq includes, but is not limited to, Drop-seq, Seq-Well, InDrop and 1Cell Bio. RNA-seq methods also include, but are not limited to, smart-seq2, TruSeq, CEL-Seq, STRT, ChIRP-Seq, GRO-Seq, CLIP-Seq, Quartz-Seq, or any other similar method known in the art (see, e.g., “Sequencing Methods Review” Illumina® Technology, Sequencing Methods Review available at illumina.com.

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In some embodiments, Drop-sequence methods or Drop-seq are contemplated for the present invention and can be used. Cells come in different types, sub-types and activity states, which are classify based on their shape, location, function, or molecular profiles, such as the set of RNAs that they express. RNA profiling is in principle particularly informative, as cells express thousands of different RNAs. Approaches that measure for example the level of every type of RNA have until recently been applied to “homogenized” samples—in which the contents of all the cells are mixed together. Methods to profile the RNA content of tens and hundreds of thousands of individual human cells have been recently developed, including from brain tissues, quickly and inexpensively. To do so, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from. See, e.g., methods of Macosko et al., 2015, Cell 161, 1202-1214 and Klein et al., 2015, Cell 161, 1187-1201 are contemplated for the present invention.

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets or microwells. The ability to conduct reactions in microdroplets depends on being able to merge different sample fluids and different microdroplets. See, e.g., US Patent Publication No. 20120219947. See also international patent application serial no. PCT/US2014/058637 for disclosure regarding a microfluidic laboratory on a chip.

Droplet/microwell microfluidics offers significant advantages for performing high-throughput screens and sensitive assays. Droplets allow sample volumes to be significantly reduced, leading to concomitant reductions in cost. Manipulation and measurement at kilohertz speeds enable up to 108 discrete biological entities (including, but not limited to, individual cells or organelles) to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

Drop-Sequence methods and apparatus provides a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells.

InDrop™, also known as in-drop seq, involves a high-throughput droplet-microfluidic approach for barcoding the RNA from thousands of individual cells for subsequent analysis by next-generation sequencing (see, e.g., Klein et al., Cell 161(5), pp 1187-1201, 21 May 2015). Specifically, in in-drop seq, one may use a high diversity library of barcoded primers to uniquely tag all DNA that originated from the same single cell. Alternatively, one may perform all steps in drop.

Well-based biological analysis or Seq-Well is also contemplated for the present invention. The well-based biological analysis platform, also referred to as Seq-well, facilitates the creation of barcoded single-cell sequencing libraries from thousands of single cells using a device that contains 100,000 40-micron wells. Importantly, single beads can be loaded into each microwell with a low frequency of duplicates due to size exclusion (average bead diameter 35 μm). By using a microwell array, loading efficiency is greatly increased compared to drop-seq, which requires poisson loading of beads to avoid duplication at the expense of increased cell input requirements. Seq-well, however, is capable of capturing nearly 100% of cells applied to the surface of the device.

Seq-well is a methodology which allows attachment of a porous membrane to a container in conditions which are benign to living cells. Combined with arrays of picoliter-scale volume containers made, for example, in PDMS, the platform provides the creation of hundreds of thousands of isolated dialysis chambers which can be used for many different applications. The platform also provides single cell lysis procedures for single cell RNA-seq, whole genome amplification or proteome capture; highly multiplexed single cell nucleic acid preparation (about 100× increase over current approaches); highly parallel growth of clonal bacterial populations thus providing synthetic biology applications as well as basic recombinant protein expression; selection of bacterial that have increased secretion of a recombinant product possible product could also be small molecule metabolite which could have considerable utility in chemical industry and biofuels; retention of cells during multiple microengraving events; long term capture of secreted products from single cells; and screening of cellular events. Principles of the present methodology allow for addition and subtraction of materials from the containers, which has not previously been available on the present scale in other modalities.

Seq-Well also enables stable attachment (through multiple established chemistries) of porous membranes to PDMS nanowell devices in conditions that do not affect cells. Based on requirements for downstream assays, amines are functionalized to the PDMS device and oxidized to the membrane with plasma. With regard to general cell culture uses, the PDMS is amine functionalized by air plasma treatment followed by submersion in an aqueous solution of poly(lysine) followed by baking at 80° C. For processes that require robust denaturing conditions, the amine must be covalently linked to the surface. This is accomplished by treating the PDMS with air plasma, followed by submersion in an ethanol solution of amine-silane, followed by baking at 80° C., followed by submersion in 0.2% phenylene diisothiocyanate (PDITC) DMF/pyridine solution, followed by baking, followed by submersion in chitosan or poly(lysine) solution. For functionalization of the membrane for protein capture, membrane can be amine-silanized using vapor deposition and then treated in solution with NHS-biotin or NHS-maleimide to turn the amine groups into the crosslinking species.

After functionalization, the device is loaded with cells (bacterial, mammalian or yeast) in compatible buffers. The cell-laden device is then brought in contact with the functionalized membrane using a clamping device. A plain glass slide is placed on top of the membrane in the clamp to provide force for bringing the two surfaces together. After an hour incubation, as one hour is a preferred time span, the clamp is opened and the glass slide is removed. The device can then be submerged in any aqueous buffer for days without the membrane detaching, enabling repetitive measurements of the cells without any cell loss. The covalently-linked membrane is stable in many harsh buffers including guanidine hydrochloride which can be used to robustly lyse cells. If the pore size of the membrane is small, the products from the lysed cells will be retained in each well. The lysing buffer can be washed out and replaced with a different buffer which allows binding of biomolecules to probes preloaded in the wells. The membrane can then be removed, enabling addition of enzymes to reverse transcribe or amplify nucleic acids captured in the wells after lysis. Importantly, the chemistry enables removal of one membrane and replacement with a membrane with a different pore size to enable integration of multiple activities on the same array.

As discussed, while the platform has been optimized for the generation of individually barcoded single-cell sequencing libraries following confinement of cells and mRNA capture beads (Macosko, et al. Cell. 2015 May 21; 161(5): 1202-1214), it is capable of multiple levels of data acquisition. The platform is compatible with other assays and measurements performed with the same array. For example, profiling of human antibody responses by integrated single-cell analysis is discussed with regard to measuring levels of cell surface proteins (Ogunniyi, A. O., B. A. Thomas, T. J. Politano, N. Varadarajan, E. Landais, P. Poignard, B. D. Walker, D. S. Kwon, and J. C. Love, “Profiling Human Antibody Responses by Integrated Single-Cell Analysis” Vaccine, 32(24), 2866-2873.) The authors demonstrate a complete characterization of the antigen-specific B cells induced during infections or following vaccination, which enables and informs one of skill in the art how interventions shape protective humoral responses. Specifically, this disclosure combines single-cell profiling with on-chip image cytometry, microengraving, and single-cell RT-PCR.

The invention provides a method for creating a single-cell sequencing library comprising: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells; and preparing and sequencing a single composite RNA-Seq library.

The invention provides a method for preparing uniquely barcoded mRNA capture microbeads, which has a unique barcode and diameter suitable for microfluidic devices comprising: 1) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides of length two or more bases; 2) repeating this process a large number of times, at least two, and optimally more than twelve, such that, in the latter, there are more than 16 million unique barcodes on the surface of each bead in the pool. (See http://www.ncbi.nlm.nih.gov/pmc/articles/PMC206447).

In another embodiment, the invention encompasses making beads specific to the panel of desired mutations or mutations plus mRNA and a capture of both. In one embodiment, one or more mutation hot spots may be near the 3′ end.

Generally, the invention provides a method for preparing a large number of beads, particles, microbeads, nanoparticles, or the like with unique nucleic acid barcodes comprising performing polynucleotide synthesis on the surface of the beads in a pool-and-split fashion such that in each cycle of synthesis the beads are split into subsets that are subjected to different chemical reactions; and then repeating this split-pool process in two or more cycles, to produce a combinatorially large number of distinct nucleic acid barcodes. Invention further provides performing a polynucleotide synthesis wherein the synthesis may be any type of synthesis known to one of skill in the art for “building” polynucleotide sequences in a step-wise fashion. Examples include, but are not limited to, reverse direction synthesis with phosphoramidite chemistry or forward direction synthesis with phosphoramidite chemistry. Previous and well-known methods synthesize the oligonucleotides separately then “glue” the entire desired sequence onto the bead enzymatically. Applicants present a complexed bead and a novel process for producing these beads where nucleotides are chemically built onto the bead material in a high-throughput manner. Moreover, Applicants generally describe delivering a “packet” of beads which allows one to deliver millions of sequences into separate compartments and then screen all at once.

The invention further provides an apparatus for creating a single-cell sequencing library via a microfluidic system, comprising: an oil-surfactant inlet comprising a filter and a carrier fluid channel, wherein said carrier fluid channel further comprises a resistor; an inlet for an analyte comprising a filter and a carrier fluid channel, wherein said carrier fluid channel further comprises a resistor; an inlet for mRNA capture microbeads and lysis reagent comprising a filter and a carrier fluid channel, wherein said carrier fluid channel further comprises a resistor; said carrier fluid channels have a carrier fluid flowing therein at an adjustable or predetermined flow rate; wherein each said carrier fluid channels merge at a junction; and said junction being connected to a mixer, which contains an outlet for drops.

A mixture comprising a plurality of microbeads adorned with combinations of the following elements: bead-specific oligonucleotide barcodes created by the discussed methods; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). In an embodiment, the individual oligonucleotide molecules on the surface of any individual microbead contain all three of these elements, and the third element includes both oligo-dT and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 1251, 3H, and 1311), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 di sulfonate; N-(4-anilino-l-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

The invention discussed herein enables high throughput and high-resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. Disclosed embodiments provide 10⁴ to 10⁵ single cells in droplets which can be processed and analyzed in a single run.

To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination.

Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets.

Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be affected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another embodiment, the expansion and/or contraction of the liquid reservoir may be combined with other flow-controlling devices and methods, e.g., as discussed herein. Non-limiting examples of devices able to cause the expansion and/or contraction of a liquid reservoir include pistons.

Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example, if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform.

Droplets formed in oil without surfactant are not stable to permit coalescence, so surfactants must be dissolved in the oil that is used as the continuous phase for the emulsion library. Surfactant molecules are amphiphilic-part of the molecule is oil soluble, and part of the molecule is water soluble. When a water-oil interface is formed at the nozzle of a microfluidic chip for example in the inlet module discussed herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification.

A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as discussed in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

A bead-based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids.

Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell.

Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

The droplets comprised within the emulsion libraries of the present invention may be contained within an immiscible oil which may comprise at least one fluorosurfactant. In some embodiments, the fluorosurfactant comprised within immiscible fluorocarbon oil is a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays discussed herein. Fluids (e.g., aqueous fluids, immiscible oils, etc.) and other surfactants that may be utilized in the droplet libraries of the present invention are discussed in greater detail herein.

The present invention provides an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library.

For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In one example, the cells may comprise cancer cells of a tissue biopsy, and each cell type is encapsulated to be screened for genomic data or against different drug therapies. Another example is that 1011 or 1015 different type of bacteria; each having a different plasmid spliced therein, are encapsulated. One example is a bacterial library where each library element grows into a clonal population that secretes a variant on an enzyme.

In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a LacZ plasmid DNA was encapsulated at a concentration of 20 fM after two hours of incubation such that there was about one gene in 40 droplets, where 10 μm droplets were made at 10 kHz per second. Formation of these libraries rely on limiting dilutions.

Methods of the invention involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety.

In certain embodiments, the carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional patent application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional patent application entitled “Systems and Methods for Droplet Tagging” filed Sep. 21, 2012.

Applications of the disclosed device may include use for the dynamic generation of molecular barcodes (e.g., DNA oligonucleotides, fluorophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost-effective manner. Disclosed embodiments provide a high throughput and high-resolution delivery of reagents to individual emulsion droplets that may contain cells, nucleic acids, proteins, etc. through the use of monodisperse aqueous droplets that are generated one by one in a microfluidic chip as a water-in-oil emulsion. Hence, the invention proves advantageous over prior art systems by being able to dynamically track individual cells and droplet treatments/combinations during life cycle experiments. Additional advantages of the disclosed invention provide an ability to create a library of emulsion droplets on demand with the further capability of manipulating the droplets through the disclosed process(es). Disclosed embodiments may, thereby, provide dynamic tracking of the droplets and create a history of droplet deployment and application in a single cell-based environment.

Droplet generation and deployment is produced via a dynamic indexing strategy and in a controlled fashion in accordance with disclosed embodiments of the present invention. Disclosed embodiments of the microfluidic device discussed herein provides the capability of microdroplets that be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays.

The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In certain embodiments, tagmentation is used to introduce adaptor sequences to genomic DNA in regions of accessible chromatin (e.g., between individual nucleosomes) (see, e.g., US20160208323A1; US20160060691A1; WO2017156336A1; and Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7). In certain embodiments, tagmentation is applied to bulk samples or to single cells in discrete volumes.

The 3′ barcoded libraries can be used in the methods as described herein to provide enriched libraries containing transcripts of interest that are not as abundant or accessible in the original single cell RNAseq libraries. Other Seq-Well embodiments that may be used with the current invention are described in PCT Publication WO2019/084058.

Optionally Treating with USER Enzyme and Amplifying

In some embodiments, the primers for amplifying in in a first PCR amplification comprise USER sequences, and further comprising treating the first PCR product with USER enzyme, thereby generating a circularized product.

The steps include cleaving the dU residue by addition of a uracil-specific excision reagent (“USER®”) enzyme/T4 ligase to generate long complementary sticky ends to mediate efficient circularization and ligation, which now places the barcode and the 5′ edge of the transcript sequence set in the primer extension in close proximity, thereby bringing the cell barcode within 100 bases of any desired sequence in the transcript.

Following treating with USER enzyme, the step of amplifying the circularized product in a second polymerase chain reaction with one or more primers, wherein the one or primers comprise a library barcode and/or additional sequencing adapters can be conducted.

In some embodiments, the method can then include more than one PCR steps with transcript specific primers, that can include adaptor sequences, and preferably uses nested PCR reactions where the final PCR reaction sets the 3′ edge of the transcript sequence of the final sequencing construct. The final sequencing library can be utilized in several ways, including sequencing of the transcript sequence, or at some desired location in the transcript sequence.

Circularization without Enrichment

In one embodiment, the methods disclosed herein provide a protocol that eliminates need for enrichment in a scalable process. An exemplary embodiment can provide for amplification of all variable regions of a T-cell receptor. The methods described herein can be advantageously be used for the amplification of regions not well characterized in RNA seq libraries. The steps include providing an RNAseq library, in some preferred embodiments, a SeqWell library. The starting library comprises a plurality of nucleic acids with each nucleic acid comprising a gene, a unique molecular identifier (UMI) and a cell barcode (cell BC) flanked by universal sequences.

In an embodiment, the method comprises conducting primer extension on a nucleic acid in the library with one or more 5′ primers with each primer comprising a sequence complementary to a desired transcript and the universal sequence of the nucleic acid, thereby replicating one or more desired transcripts and setting a 5′ edge of one or more desired transcript sequences in one or more final sequencing constructs; amplifying the replicated one or more desired transcript sequences with universal primers having complementary sequences on 5′ ends of the universal primers followed by a deoxy-uracil residue to form an amplicon; and ligating the amplicons by reacting the amplicons with a uracil-specific excision reagent enzyme, thereby cleaving the amplicon at the deoxy-uracil residues resulting in sticky ends that mediate circularization.

Additional steps of amplifying by PCR may be performed. In these instances, primers complementary to a transcript of interest. In some preferred embodiments, at least two PCR steps are performed in a nested PCR using two sets of transcript specific primers complementary to a transcript of interest. As described previously, the primers may comprise adaptor sequences. In one embodiment, at least one set of the two sets of transcript specific primers comprise adaptor sequences, thereby yielding a final sequencing library of final sequencing constructs. In an embodiment, the last PCR step sets a 3′ edge of the transcript sequence of the final construct. In some embodiments, the sequencing step utilizes primers complementary to the 3′ set and 5′ set edges of the final sequencing construct. The sequencing step can utilize a primer binding to a desired location in the final sequencing construct to drive a sequencing read at the desired location in the final sequencing construct, as described elsewhere herein.

The embodiments disclosed herein method works particularly well for libraries where a subset of the transcripts of interest are more than 1 kb away from the cell barcode. Particularly, variable regions of T-cell receptors can be used in the current methods. Accordingly, the transcript of interest can be in a T cell or a B cell, in some embodiments, in a T cell receptor, a B cell receptor or a CAR-T cell. Advantageously, the embodiment can comprise use of a pool of primers that, in an embodiment targeting variable regions, may target all variable regions. The sequencing method may also determine SNPs in the single cell.

### Determining Genotype

Determining the genotype of the cell may be accomplished by identifying the UMI and cell BC, thereby distinguishing the cells by genotype, or expressed DNA sequences, such as mutations, translocations, insertions/deletions (indels), etc. In one embodiment, the nucleic acids comprise a tag that is a molecule that can be affinity selected such as, but not limited to, a small protein, peptide, nucleic acid. Advantageously, the tag is a biotin tag. The enriched libraries provided by the methods may be further distinguished or manipulated, including by subjecting to sequencing.

In addition to next-generation sequencing, long read/third-generation sequencing is also contemplated for use in the presently disclosed subject matter. Third-generation sequencing reads nucleotide sequences at the single molecule level. In some embodiments, third-generation sequencing is used when long reads are desired, and can be used, in some instances, instead of next-generation sequencing technologies in desired applications. In particular embodiments, nanopore sequencing or single molecule real time sequencing (SMRT) is used for third-generation sequencing. Nanopore technology libraries are generated by end-repair and sequencing adapter ligation, and, as such, allows for versatility in the sequencing adapters utilized in the PCR reaction. Accordingly, in some instances, when nanopore sequencing is utilized, the ‘sequencing adapters’ in the first PCR reaction is any adapter that allows for a second PCR with common primers. Exemplary nanopore technology that can be used for long reads can be found, for example, using Oxford Nanopore technology, available at nanoporetech.com. Long-read sequencing can also utilize SMRT sequencing which enables single-molecule resolution through the use of nucleotides uniquely labeled with a fluorophore, and observing a single DNA polymerase molecule while synthesizing a complementary DNA in a replication reaction to allow for single molecule resolution. tallows production of a natural DNA strand using the labeled nucleotides. In some instances, when third-generation sequencing will be used, additional amplification can be performed to generate sufficient material.

### Distinguishing Cells by Genotype

A method of distinguishing cells by genotype may, in some embodiments comprise constructing a library as discussed herein that comprises a plurality of nucleic acids wherein each nucleic acid comprises a gene, a unique molecular identifier (UMI) and a cell barcode (cell BC) flanked by sequencing adapters at the 5′ and 3′ end. In particular embodiments, each nucleic acid comprises the orientation: 5′-sequencing adapter-cell barcode-UMI-UUUUUUU-mRNA-3′. Amplifying each nucleic acid in the library to create a whole transcriptome amplified (WTA) RNA by reverse transcription can be performed with a primer comprising a sequence adapter to provide a reverse transcribed product. The steps provide amplifying the reverse transcribed product by PCR amplification with primers that bind both sequence adapters and adding a library barcode and optionally additional sequence adapters to generate a first PCR product. The genotype of the cell can be performed as discussed elsewhere, including identifying the UMI and library barcode, thereby distinguishing the cells by genotype.

### Reverse Transcribing

In some embodiments, such as determining a cell signature or constructing a library, reverse transcribing can be included. In some embodiments, reverse transcription can include amplification of a reverse transcribed product. In specific embodiments, the amplification reaction mixture may further comprise a polymerase. Subsequent to melting and hybridization with a primer, the nucleic acid is subjected to a polymerization step. A DNA polymerase is selected if the nucleic acid to be amplified is DNA. When the initial target is RNA, a reverse transcriptase may first be used to copy the RNA target into a cDNA molecule and the cDNA is then further amplified by a selected DNA polymerase. The DNA polymerase acts on the target nucleic acid to extend the primers hybridized to the nucleic acid templates in the presence of four dNTPs to form primer extension products complementary to the nucleotide sequence on the nucleic acid template.

### RNA-Seq/Single Cell Sequencing

As described above, in some embodiments, gene expression can be determined using an RNA-seq-based method. In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-6′73, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### MS Methods

The cell signature can, in some embodiments, be identified by detecting biomarker by a mass spectrometry method. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

In some embodiments, a method of detecting cell signature can include performing an immunoassay. Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

In some embodiments, a method of detecting cell signature can include performing an hybridization assay. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B. V. (1993) and Kricka, “Nonisotopic DNA Probe Techniques”, Academic Press, San Diego, Calif. (1992).

Detecting mtDNA Heteroplasmy

As previously described, detecting the cell signature and/or detecting mtDNA heteroplasmy. mtDNA heteroplasmy can be evaluated, detected, and/or measured by any suitable method. In some embodiments, detecting mtDNA heteroplasmy can include isolating and optionally enriching mtDNA from a cell or cell population, tissue, or other biological sample containing mtDNA. In some embodiments, detecting DNA can include a polynucleotide sequencing method. In some embodiments, detecting mtDNA heteroplasmy can include an RNA sequencing method. In some embodiments, detecting mtDNA heteroplasmy can include a DNA sequencing method. In some embodiments, detecting mtDNA heteroplasmy can include a direct sequencing method of mtDNA. In some embodiments, detecting mtDNA heteroplasmy can include an indirect sequencing method of mtDNA. In this context and as used herein, “direct sequencing” refers to methods that sequence mtDNA directly through mtDNA isolated and/or enriched from total cellular DNA. In this context and as used herein, “indirect sequencing” can refers to methods to obtain mitochondrial DNA sequencings as by-products of other types of high-throughput sequencing methods. Direct and indirect methods both have advantages. One of ordinary skill in the art will appreciate the different features and advantages of methods and choose accordingly.

In addition to any methods described elsewhere herein, suitable methods of isolating and/or enriching mtDNA will be appreciated by one of ordinary skill in the art and can include, for example, any of those as set forth in Koref et al. Mitochondrion. 2019. 46:302-306 (see e.g. Methods and Supplementary materials at e.g. “mtDNA Enrichment”) or via a commercially available enrichment kits (e.g. those described and used in the methods of Ancora M. 2017 and Marquis et al., 2017). In some embodiments, enrichment can be accomplished by PCR amplification-based method. In some embodiments, isolation and/or enrichment of mtDNA can be accomplished by generating several overlapping PCR amplicons (typically 100-2000 base-pairs long) (see e.g. Payne et al. Nat. Genet. 2011. 43(8): 806-810 and Payne et al. Methods Mol. Biol. 2015; 1264:67-76). In some embodiments, isolation and/or enrichment of mtDNA can be accomplished using long-range PCR (typically producing one or two overlapping large amplicons) (see e.g. Kang et al. 2016. Nature. 540 (270-+); Rygiel et al. 2016. Nucleic Acids Res, 44:5313-5329; and van der Walt et al., 2012. Eur. J. Hum. Genet. 20:650-656). In some embodiments, isolation and/or enrichment of mtDNA can be accomplished by generating the mtDNA genome as one large amplicon (see e.g. Zhang et al. 2012. Clin. Chem. 58:1322-1331 and Cui et al., Genet Med. 2013 May; 15(5):388-94). These commercially available kits typically rely on multiple displacement amplification that produce a series of overlapping fragments. Example kits include, but are not limited to, those by Qiagen SAbiosciences (e.g. RePLI-g Mitochondrial DNA Kit) and Integrated DNA Technologies (a solution phase capture based-kit utilizing IDT's xGen Lockdown probes). In some embodiments, isolation and/or enrichment of mtDNA can include density gradient separation (e.g. ultra-centrifugation in CsCL density gradients and others). In some embodiments, isolation/enrichment can be accomplished using a hybridization-based technique (e.g. a microarray hybridization enrichment method as exemplified in Vasta et al., Genome Med. 2009 Oct. 23; 1(10):100 and Guo at al. Mutat Res. 2012 May 15; 744(2):154-60), primer capturing as exemplified in He et al., Nature. 2010 Mar. 25; 464(7288):610-4 and Sosa et al. PLoS Comput Biol. 2012; 8(10):e1002737).

In some embodiments, the mtDNA can be extracted from other types of high-throughput sequencing data such as exome and whole genome sequencing data. In exome data, a significant amount of reads can align to the mitochondrial genome (around about 1-5%), even if not the intended target (see e.g. Samuels et al., Trends Genet. 2013 October; 29(10):593-9; Larmen et al. Proc Natl Acad Sci USA. 2012 Aug. 28; 109(35):14087-91; Picardi and Pesole. Nat Methods. 2012 May 30; 9(6):523-4). The average coverage of the mitochondrial genome from exome sequencing is about 100 (Picardi and Pesole. 2012), although this can vary upon tissue type examined due to differences between mitochondrial copy number in different tissue/cell types.

In some embodiments, mtDNA or enriched mtDNA, can be sequenced using any suitable DNA sequencing method. Basic DNA sequencing methods suitable for use in some embodiments include those based on chemical degradation, primer extension/chain termination-based methods (e.g. Sanger sequencing), and shot-gun sequencing/analysis and others. High-throughput (both short-read and long-read) sequencing methods suitable for use in some embodiments include stepwise or “base-by-base” based methods, pyrosequencing, single molecule real-time sequencing, ion semiconductor sequencing, sequencing by synthesis, colony sequencing (used in Illumina's Hi-Seq sequencing machines), combinatorial probe anchor synthesis, sequencing by ligation, nanopore sequencing, genapsys sequencing, polony sequencing, nanoball sequencing, ATAC-Seq, DNAse-Seq, FAIRE-Seq, and massively parallel signature sequencing (MPSS), sequencing by hybridization and the like. Other suitable sequencing methods include, but are not limited to, microfluidic-based sequencing, microscopy based sequencing techniques (e.g. transmission electron microscopy DNA sequencing), RNAP (RNA polymerase)-based sequencing, and tunneling current-based sequencing. Suitable sequencing methods include single cell sequencing methods.

Suitable RNA sequencing methods can be used to evaluate mtDNA. Suitable RNA sequencing methods include, but are not limited to, Sanger processing of Expressed Sequence Tag libraries, chemical tag-based methods (e.g. serial analysis of gene expression) and basic or next generation sequencing of cDNA (notably RNA-Seq). In some embodiments, the RNA sequencing method can be a single cell RNA sequencing technique (e.g. single-cell RNA-seq). In some embodiments, the next generation sequencing methods performed in connection with an RNA-Seq method can be “base-by-base” based methods, pyrosequencing, single molecule real-time sequencing, ion semiconductor sequencing, sequencing by synthesis, colony sequencing (used in Illumina's Hi-Seq sequencing machines), combinatorial probe anchor synthesis, sequencing by ligation, nanopore sequencing, genapsys sequencing, polony sequencing, nanoball sequencing, ATAC-Seq, DNAse-Seq, FAIRE-Seq, and massively parallel signature sequencing (MPSS), sequencing by hybridization and the like. In some embodiments, the sequencing method comprises single cell RNA sequencing and/or mitochondrial DNA single cell ATAC-seq (mtscATAC-seq). Other suitable sequencing methods to detect mtDNA heteroplasmy are described elsewhere herein.

mtDNA sequencing data can be analyzed by any suitable method, which will be appreciated by one of ordinary skill in the art. In some embodiments, the mtDNA sequence generated can be compared to a suitable reference sequence, including but not limited to, the revised Cambridge Reference Sequence (rCRS), the sequence given GenBank Accession No. NM_012920.1 (see e.g., Koref et al. Mitochondrion. 2019. 46:302-306; Ancora M. Complete sequence of human mitochondrial DNA obtained by combining multiple displacement amplification and next-generation sequencing on a single oocyte. Mitochondrial DNA A. 2017; 28:180-181; Dolle, C. et al. Defective mitochondrial DNA homeostasis in the substantia nigra in Parkinson disease. Nature Communications7, doi:Artn 13548 10.1038/Ncomms13548 (2016); Kang E. J. Mitochondrial replacement in human oocytes carrying pathogenic mitochondrial DNA mutations. Nature. 2016; 540 (270-+); Kang E. J. Age-related accumulation of somatic mitochondrial DNA mutations in adult-derived human iPSCs. Cell Stem Cell. 2016; 18:625-636; Marquis, J. et al. MitoRS, a method for high throughput, sensitive, and accurate detection of mitochondrial DNA heteroplasmy. Bmc Genomics18, doi:Artn 326 10.1186/S12864-017-3695-5 (2017); Payne B. A., Cree L., Chinnery P. F. Single-cell analysis of mitochondrial DNA. Methods Mol. Biol. 2015; 1264:67-76; Rygiel K. A. Complex mitochondrial DNA rearrangements in individual cells from patients with sporadic inclusion body myositis. Nucleic Acids Res. 2016; 44:5313-5329; van der Walt E. M. Characterization of mtDNA variation in a cohort of south African paediatric patients with mitochondrial disease. Eur. J. Hum. Genet. 2012; 20:650-656; and Yamada M. Genetic drift can compromise mitochondrial replacement by nuclear transfer in human oocytes. Cell Stem Cell. 2016; 18:749-754).

### Mutations

In some embodiments, detecting mtDNA heteroplasmy includes detecting one or more mutations the mtDNA. In some embodiments, at least one of the one or more mutations are pathogenic.

In some embodiments, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 1) repeats at positions 305-314 and/or 956-965, deletion at positions from 8,469-13,447, 4,308-14,874, and/or 4,398-14,822, 96 lins/delC, the mitochondrial common deletion (e.g. mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations. In some embodiments, the mutation can be a mutation shown in any of Tables 1-5 or a combination thereof.

TABLE 1
Exemplary mtDNA mutations.

| A | B | C | D | E | F | G | H | I | J | K | L |
|---|---|---|---|---|---|---|---|---|---|---|---|
| 582 | MT-TF | Mitochondrial myopathy | T582C | tRNA Phe | − | + | Reported | 32.90% | 0% | 0 | 2 |
| | | | | | | | | | (0%) | (0) | |
| 583 | MT-TF | MELAS/MM & EXIT | G583A | tRNA Phe | − | + | Cfrm | Pathogenic | 0% | 0 | 3 |
| | | | | | | | | | (0%) | (0) | |
| 586 | MT-TF | Extrapyramidal disorder with | G586A | tRNA Phe | − | + | Reported | 89.70% | 0% | 0 | 2 |
| | | akinesia-rigidity, psychosis | | | | | | | (0%) | (0) | |
| | | and SNHL | | | | | | | | | |
| 593 | MT-TF | Nonsyndromic hearing loss | T593C | tRNA Phe | + | − | Reported | 9.80% | 0.4% | 205 | 1 |
| | | | | | | | | | (0%) | (0) | |
| 602 | MT-TF | Axial myopathy with | C602T | tRNA Phe | − | + | Reported | 85.90% | 0% | 0 | 2 |
| | | encephalopathy | | | | | | | (0%) | (0) | |
| 606 | MT-TF | Myoglobinuria | A606G | tRNA Phe | + | + | Unclear | 64.90% | 0% | 18 | 3 |
| | | | | | | | | | (0%) | (0) | |
| 608 | MT-TF | Tubulo-interstitial nephritis | A608G | tRNA Phe | + | − | Reported | 65.00% | 0% | 0 | 2 |
| | | | | | | | | | (0%) | (0) | |
| 611 | MT-TF | ERRF | G611A | tRNA Phe | − | + | Reported | 51.20% | 0% | 0 | 3 |
| | | | | | | | | | (0%) | (0) | |
| 616 | MT-TF | Maternally inherited epilepsy/ | T616C | tRNA Phe | + | + | Cfrm | Pathogenic | 0% | 1 | 2 |
| | | kidney disease | | | | | | | (0%) | (0) | |
| 616 | MT-TF | Maternally inherited epilepsy | T616G | tRNA Phe | + | + | Reported | 95.60% | 0% | 1 | 1 |
| | | | | | | | | | (0%) | (0) | |
| 617 | MT-TF | Carotid artery stenosis | G617A | tRNA Phe | − | + | Reported | 21.70% | 0% | 0 | 1 |
| | | | | | | | | | (0%) | (0) | |
| 618 | MT-TF | MM | T618C | tRNA Phe | − | + | Reported | 65.80% | 0% | 0 | 1 |
| | | | | | | | | | (0%) | (0) | |
| 618 | MT-TF | Ptosis CPEO MM & EXIT | T618G | tRNA Phe | − | + | Reported | 77.50% | 0% | 0 | 1 |
| | | | | | | | | | (0%) | (0) | |
| 622 | MT-TF | EXIT & Deafness | G622A | tRNA Phe | − | + | Reported | 41.50% | 0% | 0 | 2 |
| | | | | | | | | | (0%) | (0) | |
| 625 | MT-TF | SNHL & Epilepsy | G625A | tRNA Phe | − | + | Reported | 81.30% | 0% | 0 | 1 |
| | | | | | | | | | (0%) | (0) | |
| 628 | MT-TF | DEAF | C628T | tRNA Phe | − | + | Reported | 34.80% | 0% | 3 | 1 |
| | | | | | | | | | (0%) | (0) | |
| 636 | MT-TF | DEAF | A636G | tRNA Phe | + | − | Reported | 1.30% | 0% | 18 | 3 |
| | | | | | | | | | (0%) | (0) | |
| 641 | MT-TF | Epileptic Encephalopathy | A641T | tRNA Phe | − | + | Reported | 69.00% | 0% | 0 | 1 |
| | | | | | | | | | (0%) | (0) | |
| 642 | MT-TF | Ataxia, PEO, deafness | T642C | tRNA Phe | − | + | Reported | 67.60% | 0% | 0 | 1 |
| | | | | | | | | | (0%) | (0) | |
| 652 | MT-RNR1 | Atherosclerosis risk | G652del | 12S rRNA | − | + | Reported | N/A | 0% | 0 | 2 |
| | | | | | | | | | (0%) | (0) | |
| 652 | MT-RNR1 | Atherosclerosis study | G652GG | 12S rRNA | − | − | Reported | N/A | 0% | 0 | 1 |
| | | | | | | | | | (0%) | (0) | |
| 663 | MT-RNR1 | Coronary atherosclerosis risk | A663G | 12S rRNA | + | − | Reported | N/A | 2.8% | 1404 | 1 |
| | | | | | | | | | (0%) | (0) | |
| 669 | MT-RNR1 | DEAF | T669C | 12S rRNA | + | − | Reported | N/A | 0.2% | 87 | 4 |
| | | | | | | | | | (0%) | (0) | |
| 721 | MT-RNR1 | Possibly LVNC-associated | T721C | 12S rRNA | + | − | Reported | N/A | 0.2% | 125 | 1 |
| | | | | | | | | | (0%) | (0) | |
| 735 | MT-RNR1 | DEAF | A735G | 12S rRNA | − | − | Reported | N/A | 0.1% | 23 | 1 |
| | | | | | | | | | (0%) | (0) | |
| 745 | MT-RNR1 | DEAF-associated | A745G | 12S rRNA | + | − | Reported | N/A | 0.1% | 32 | 1 |
| | | | | | | | | | (0%) | (0) | |
| 750 | MT-RNR1 | SZ-associated | A750A | 12S rRNA | + | − | Reported | N/A | 1.7% | 864 | 3 |
| | | | | | | | | | (0%) | (0) | |
| 792 | MT-RNR1 | Increased risk of | C792T | 12S rRNA | + | − | Reported | N/A | 0% | 4 | 1 |
| | | nonsyndromic deafness | | | | | | | (0%) | (0) | |
| 801 | MT-RNR1 | DEAF-associated | A801G | 12S rRNA | + | − | Reported | N/A | 0% | 6 | 1 |
| | | | | | | | | | (0%) | (0) | |
| 827 | MT-RNR1 | DEAF | A827G | 12S rRNA | + | − | Conflicting | N/A | 2.5% | 1276 | 16 |
| | | | | | | | reports | | (0%) | (0) | |
| 839 | MT-RNR1 | DEAF-associated | A839G | 12S rRNA | + | − | Reported | N/A | 0% | 6 | 1 |
| | | | | | | | | | (0%) | (0) | |
| 850 | MT-RNR1 | Possibly LVNC-associated | T850C | 12S rRNA | + | − | Reported | N/A | 0.2% | 123 | 1 |
| | | | | | | | | | (0%) | (0) | |
| 856 | MT-RNR1 | LHON helper/AD/DEAF- | A856G | 12S rRNA | + | − | Reported | N/A | 0% | 19 | 3 |
| | | associated | | | | | | | (0%) | (0) | |
| 869 | MT-RNR1 | found in 1 HCM patient | C869T | 12S rRNA | + | − | Reported | N/A | 0.1% | 70 | 1 |
| | | | | | | | | | (0%) | (0) | |
| 921 | MT-RNR1 | Possibly LVNC-associated | T921C | 12S rRNA | + | − | Reported | N/A | 0.8% | 397 | 2 |
| | | | | | | | | | (0%) | (0) | |
| 960 | MT-RNR1 | Possibly DEAF-associated | C960del | 12S rRNA | + | − | Reported | N/A | 0% | 0 | 1 |
| | | | | | | | | | (0%) | (0) | |
| 960 | MT-RNR1 | Possibly DEAF-associated | C960CC | 12S rRNA | + | − | Reported | N/A | 0.6% | 282 | 4 |
| | | | | | | | | | (0%) | (0) | |
| 961 | MT-RNR1 | DEAF, possibly LVNC- | T961C | 12S rRNA | + | − | Unclear | N/A | 0.9% | 442 | 7 |
| | | associated | | | | | | | (0%) | (0) | |
| 961 | MT-RNR1 | DEAF/AD-associated/ | T961delT+/- | 12S rRNA | + | + | Unclear | N/A | 0% | 0 | 21 |
| | | intellectual disability | C(n)ins | | | | | | (0%) | (0) | |
| 961 | MT-RNR1 | Possibly DEAF-associated | T961G | 12S rRNA | + | − | Unclear | N/A | 0.4% | 189 | 5 |
| | | | | | | | | | (0%) | (0) | |
| 961 | MT-RNR1 | DEAF | T961TC | 12S rRNA | + | − | Unclear | N/A | 0% | 0 | 13 |
| | | | | | | | | | (0%) | (0) | |
| 988 | MT-RNR1 | Possible DEAF risk factor | G988A | 12S rRNA | − | − | Reported | N/A | 0.1% | 39 | 1 |
| | | | | | | | | | (0%) | (0) | |
| 990 | MT-RNR1 | DEAF | T990C | 12S rRNA | + | − | Reported | N/A | 0.1% | 33 | 1 |
| | | | | | | | | | (0%) | (0) | |
| 1005 | MT-RNR1 | DEAF | T1005C | 12S rRNA | + | − | Unclear | N/A | 0.5% | 230 | 5 |
| | | | | | | | | | (0%) | (0) | |
| 1027 | MT-RNR1 | DEAF-associated | A1027G | 12S rRNA | + | − | Reported | N/A | 0% | 14 | 1 |
| | | | | | | | | | (0%) | (0) | |
| 1095 | MT-RNR1 | SNHL | T1095C | 12S rRNA | + | + | Unclear | N/A | 0.1% | 62 | 15 |
| | | | | | | | | | (0%) | (0) | |
| 1116 | MT-RNR1 | DEAF | A1116G | 12S rRNA | + | − | Reported | N/A | 0% | 10 | 2 |
| | | | | | | | | | (0%) | (0) | |
| 1180 | MT-RNR1 | Possibly DEAF-associated | T1180G | 12S rRNA | + | − | Reported | N/A | 0% | 0 | 2 |
| | | | | | | | | | (0%) | (0) | |
| 1192 | MT-RNR1 | DEAF-associated | C1192A | 12S rRNA | + | − | Reported | N/A | 0% | 8 | 2 |
| | | | | | | | | | (0%) | (0) | |
| 1192 | MT-RNR1 | DEAF-associated | C1192T | 12S rRNA | + | − | Reported | N/A | 0% | 12 | 1 |
| | | | | | | | | | (0%) | (0) | |
| 1226 | MT-RNR1 | Possibly DEAF-associated | C1226G | 12S rRNA | + | − | Reported | N/A | 0% | 0 | 2 |
| | | | | | | | | | (0%) | (0) | |
| 1291 | MT-RNR1 | DEAF | T1291C | 12S rRNA | + | − | Unclear | N/A | 0.1% | 53 | 3 |
| | | | | | | | | | (0%) | (0) | |
| 1310 | MT-RNR1 | DEAF-associated | C1310T | 12S rRNA | + | − | Reported | N/A | 0.1% | 37 | 1 |
| | | | | | | | | | (0%) | (0) | |
| 1331 | MT-RNR1 | DEAF-associated | A1331G | 12S rRNA | + | − | Reported | N/A | 0% | 10 | 1 |
| | | | | | | | | | (0%) | (0) | |
| 1349 | MT-RNR1 | DEAF | T1349G | 12S rRNA | − | + | Reported | N/A | 0% | 0 | 1 |
| | | | | | | | | | (0%) | (0) | |
| 1374 | MT-RNR1 | DEAF-associated | A1374G | 12S rRNA | + | − | Reported | N/A | 0% | 1 | 2 |
| | | | | | | | | | (0%) | (0) | |
| 1391 | MT-RNR1 | found in 1 HCM patient | T1391C | 12S rRNA | + | − | Reported | N/A | 0.3% | 132 | 1 |
| | | | | | | | | | (0%) | (0) | |
| 1420 | MT-RNR1 | DEAF | T1420G | 12S rRNA | + | + | Reported | N/A | 0% | 0 | 1 |
| | | | | | | | | | (0%) | (0) | |
| 1438 | MT-RNR1 | SZ-associated | A1438A | 12S rRNA | + | − | Reported | N/A | 5.2% | 2602 | 3 |
| | | | | | | | | | (0%) | (0) | |
| 1452 | MT-RNR1 | DEAF-associated | T1452C | 12S rRNA | + | − | Reported | N/A | 0.1% | 48 | 1 |
| | | | | | | | | | (0%) | (0) | |
| 1453 | MT-RNR1 | Possible DEAF risk factor | A1453G | 12SrRNA | − | − | Reported | N/A | 0.2% | 107 | 1 |
| | | | | | | | | | (0%) | (0) | |
| 1492 | MT-RNR1 | DEAF | A1492C | 12S rRNA | − | + | Reported | N/A | 0% | 0 | 1 |
| | | | | | | | | | (0%) | (0) | |
| 1494 | MT-RNR1 | DEAF | C1494T | 12S rRNA | + | − | Cfrm | N/A | 0% | 4 | 29 |
| | | | | | | | | | (0%) | (0) | |
| 1517 | MT-RNR1 | DEAF | A1517C | 12S rRNA | − | + | Reported | N/A | 0% | 0 | 1 |
| | | | | | | | | | (0%) | (0) | |
| 1537 | MT-RNR1 | DEAF; intellectual disability | C1537T | 12S rRNA | + | − | Reported | N/A | 0% | 0 | 1 |
| | | | | | | | | | (0%) | (0) | |
| 1544 | MT-RNR1 | DEAF | A1544T | 12S rRNA | + | − | Reported | N/A | 0% | 0 | 2 |
| | | | | | | | | | (0%) | (0) | |
| 1546 | MT-RNR1 | DEAF | A1546T | 12S rRNA | + | − | Reported | N/A | 0% | 0 | 1 |
| | | | | | | | | | (0%) | (0) | |
| 1554 | MT-RNR1 | DEAF | G1554A | 12S rRNA | + | − | Reported | N/A | 0% | 0 | 1 |
| | | | | | | | | | (0%) | (0) | |
| 1555 | MT-RNR1 | DEAF; autism spectrum | A1555G | 12S rRNA | + | − | Cfrm | N/A | 0.1% | 74 | 138 |
| | | intellectual disability; | | | | | | | (0%) | (0) | |
| | | possibly antiatherosclerotic | | | | | | | | | |
| 1556 | MT-RNR1 | found in 1 HCM patient | C1556T | 12S rRNA | + | − | Reported | N/A | 0% | 4 | 1 |
| | | | | | | | | | (0%) | (0) | |
| 1575 | MT-RNR1 | DEAF | T1575G | 12S rRNA | + | − | Reported | N/A | 0% | 0 | 1 |
| | | | | | | | | | (0%) | (0) | |
| 1577 | MT-RNR1 | DEAF | T1577G | 12S rRNA | − | + | Reported | N/A | 0% | 0 | 1 |
| | | | | | | | | | (0%) | (0) | |
| 1606 | MT-TV | AMDF | G1606A | tRNA Val | − | + | Cfrm | Pathogenic | 0% | 0 | 4 |
| | | | | | | | | | (0%) | (0) | |
| 1607 | MT-TV | Suspected mito disease | T1607C | tRNA Val | + | + | Reported | [symbol] | 0% | 10 | 1 |
| | | | | | | | | | (0%) | (0) | |
| 1616 | MT-TV | MPLAS | A1616G | tRNA Val | − | − | Reported | 36.70% | 0% | 0 | 1 |
| | | | | | | | | | (0%) | (0) | |
| 1624 | MT-TV | Leigh Syndrome | C1624T | tRNA Val | + | − | Reported | 68.70% | 0% | 0 | 4 |
| | | | | | | | | | (0%) | (0) | |
| 1630 | MT-TV | MNGIE-like disease/ | A1630G | tRNA Val | − | + | Cfrm | Pathogenic | 0% | 0 | 3 |
| | | MELAS | | | | | | | (0%) | (0) | |
| 1642 | MT-TV | MELAS | G1642A | tRNA Val | − | + | Reported | 74.30% | 0% | 0 | 2 |
| | | | | | | | | | (0%) | (0) | |
| 1643 | MT-TV | Late infantile onset fatal mito | A1643G | tRNA Val | + | + | Reported | 42.00% | 0% | 1 | 1 |
| | | disease | | | | | | | (0%) | (1) | |
| 1644 | MT-TV | LS/HCM/MELAS | G1644A | tRNA Val | − | + | Cfrm | Pathogenic | 0% | 0 | 4 |
| | | | | | | | | | (0%) | (0) | |
| 1644 | MT-TV | Adult Leigh Syndrome | G1644T | tRNA Val | − | + | Reported | 48.40% | 0% | 0 | 1 |
| | | | | | | | | | (0%) | (0) | |
| 1659 | MT-TV | Movement Disorder | T1659C | tRNA Val | − | + | Reported | 69.60% | 0% | 0 | 4 |
| | | | | | | | | | (0%) | (0) | |
| 2158 | MT-RNR2 | Reduced risk PD | T2158C | 16S rRNA | − | − | Reported | N/A | 0.4% | 200 | 2 |
| | | | | | | | | | (0%) | (0) | |
| 2336 | MT-RNR2 | Hypertrophic cardiomyopathy | T2336C | 16S rRNA | + | − | Reported | N/A | 0% | 0 | 2 |
| | | | | | | | | | (0%) | (0) | |
| 2352 | MT-RNR2 | Possibly LVNC-associated | T2352C | 16S rRNA | + | − | Reported | N/A | 2.6% | 1281 | 3 |
| | | | | | | | | | (0%) | (0) | |
| 2361 | MT-RNR2 | Possibly LVNC-associated | G2361A | 16S rRNA | + | − | Reported | N/A | 0.3% | 135 | 1 |
| | | | | | | | | | (0%) | (0) | |
| 2639 | MT-RNR2 | Rare mutation in a single | C2639A | 16S rRNA | + | − | Reported | N/A | 0% | 1 | 1 |
| | | POAG patient | | | | | | | (0%) | (0) | |
| 2706 | MT-RNR2 | Increased risk of T2DM in | A2706A | 16S rRNA | + | − | Reported | N/A | 21% | 10515 | 1 |
| | | haplogroup H | | | | | | | (0%) | (0) | |
| 2755 | MT-RNR2 | Possibly LVNC-associated | A2755G | 16S rRNA | + | − | Reported | N/A | 0.5% | 262 | 2 |
| | | | | | | | | | (0%) | (0) | |
| 2835 | MT-RNR2 | Rett Syndrome | C2835T | 16S rRNA | − | + | Reported | N/A | 0.1% | 58 | 2 |
| | | | | | | | | | (0%) | (0) | |
| 3010 | MT-RNR2 | Cyclic Vomiting Syndrome | G3010A | 16S rRNA | + | − | Reported | N/A | 14.4% | 7223 | 6 |
| | | with Migraine | | | | | | | (0%) | (0) | |
| 3090 | MT-RNR2 | Myopathy | G3090A | 16S rRNA | − | + | Reported | N/A | 0% | 2 | 1 |
| | | | | | | | | | (0%) | (0) | |
| 3093 | MT-RNR2 | MELAS | C3093G | 16S rRNA | − | + | Reported | N/A | 0% | 0 | 2 |
| | | | | | | | | | (0%) | (0) | |
| 3111 | MT-RNR2 | Migraine | A3111T | 16S rRNA | + | − | Reported | N/A | 0% | 6 | 1 |
| | | | | | | | | | (0%) | (0) | |
| 3196 | MT-RNR2 | ADPD | G3196A | 16S rRNA | + | + | Reported | N/A | 0% | 13 | 3 |
| | | | | | | | | | (0%) | (0) | |
| 3236 | MT-TL1 | Sporadic bilateral optic | A3236G | tRNA Leu | − | − | Reported | 37.80% | 0% | 2 | 2 |
| | | neuropathy | | (UUR) | | | | | (0%) | (0) | |
| 3242 | MT-TL1 | MM/HCM + renal tubular | G3242A | tRNA Leu | + | + | Reported | 18.50% | 0% | 0 | 5 |
| | | dysfunction | | (UUR) | | | | | (0%) | (0) | |
| 3243 | MT-TL1 | MELAS/LS/DMDF/ | A3243G | tRNA Leu | − | + | Cfrm | Pathogenic | 0% | 9 | 392 |
| | | MIDD/SNHL/CPEO/MM/ | | (UUR) | | | | | (0%) | (0) | |
| | | FSGS/ASD/ | | | | | | | | | |
| | | Cardiac + multi-organ | | | | | | | | | |
| | | dysfunction | | | | | | | | | |
| 3243 | MT-TL1 | MM/MELAS/SNHL/ | A3243T | tRNA Leu | − | + | Cfrm | Pathogenic | 0% | 0 | 6 |
| | | CPEO | | (UUR) | | | | | (0%) | (0) | |
| 3244 | MT-TL1 | MELAS | G3244A | tRNA Leu | − | + | Reported | 41.66% | 0% | 6 | 4 |
| | | | | (UUR) | | | | | (0%) | (0) | |
| 3249 | MT-TL1 | KSS | G3249A | tRNA | − | + | Reported | 39.30% | 0% | 0 | 3 |
| | | | | Leu(UUR) | | | | | (0%) | (0) | |
| 3250 | MT-TL1 | MM/CPEO | T3250C | tRNA Leu | − | + | Reported | 33.40% | 0% | 0 | 11 |
| | | | | (UUR) | | | | | (0%) | (0) | |
| 3251 | MT-TL1 | MM/MELAS with chorea- | A3251G | tRNA Leu | − | + | Reported | 43.50% | 0% | 0 | 4 |
| | | ballism | | (UUR) | | | | | (0%) | (0) | |
| 3252 | MT-TL1 | MELAS | A3252G | tRNA Leu | − | + | Reported | 29.40% | 0% | 0 | 4 |
| | | | | (UUR) | | | | | (0%) | (0) | |
| 3252 | MT-TL1 | EXIT | A3252T | tRNA Leu | − | + | Reported | 39.40% | 0% | 0 | 1 |
| | | | | (UUR) | | | | | (0%) | (0) | |
| 3253 | MT-TL1 | Maternally inherited | T3253C | tRNA Leu | + | − | Reported | 0.40% | 0% | 6 | 3 |
| | | hypertension | | (UUR) | | | | | (0%) | (0) | |
| 3254 | MT-TL1 | Gestational Diabetes (GDM) | C3254A | tRNA Leu | − | + | Reported | [symbol] | 0.1% | 26 | 1 |
| | | | | (UUR) | | | | | (0%) | (0) | |
| 3254 | MT-TL1 | MM | C3254G | tRNA Leu | − | + | Reported | 60.80% | 0% | 0 | 3 |
| | | | | (UUR) | | | | | (0%) | (0) | |
| 3254 | MT-TL1 | CPEO/poss. hypertension | C3254T | tRNA Leu | + | − | Reported | 25.30% | 0% | 17 | 5 |
| | | factor | | (UUR) | | | | | (0%) | (0) | |
| 3255 | MT-TL1 | MERRF/KSS overlap | G3255A | tRNA Leu | − | + | Reported | 75.80% | 0% | 0 | 3 |
| | | | | (UUR) | | | | | (0%) | (0) | |
| 3256 | MT-TL1 | MELAS; possible | C3256T | tRNA Leu | − | + | Cfrm | Pathogenic | 0% | 0 | 18 |
| | | atherosclerosis risk | | (UUR) | | | | | (0%) | (0) | |
| 3258 | MT-TL1 | MELAS/Myopathy | T3258C | tRNA Leu | − | + | Cfrm | Pathogenic | 0% | 1 | 5 |
| | | | | (UUR) | | | | | (0%) | (0) | |
| 3260 | MT-TL1 | MMC/MELAS | A3260G | tRNA Leu | − | + | Cfrm | Pathogenic | 0% | 0 | 10 |
| | | | | (UUR) | | | | | (0%) | (0) | |
| 3264 | MT-TL1 | DM | T3264C | tRNA Leu | − | + | Reported | 47.30% | 0% | 0 | 3 |
| | | | | (UUR) | | | | | (0%) | (0) | |
| 3271 | MT-TL1 | PEM/retinal dystrophy in | T3271del | tRNA Leu | − | + | Cfrm | Pathogenic | 0% | 0 | 3 |
| | | MELAS | | (UUR) | | | | | (0%) | (0) | |
| 3271 | MT-TL1 | MELAS/DM | T3271C | tRNA Leu | − | + | Cfrm | Pathogenic | 0% | 0 | 25 |
| | | | | (UUR) | | | | | (0%) | (0) | |
| 3273 | MT-TL1 | Ocular myopathy | T3273C | tRNA Leu | − | + | Reported | 71.20% | 0% | 0 | 3 |
| | | | | (UUR) | | | | | (0%) | (0) | |
| 3274 | MT-TL1 | Neuropsychiatric syndrome + | A3274G | tRNA Leu | − | + | Reported | 77.10% | 0% | 0 | 2 |
| | | cataract | | (UUR) | | | | | (0%) | (0) | |
| 3275 | MT-TL1 | LHON | C3275A | tRNA Leu | + | − | Reported | 2.20% | 0% | 1 | 3 |
| | | | | (UUR) | | | | | (0%) | (0) | |
| 3275 | MT-TL1 | Metabolic syndrome and | C3275T | tRNA Leu | + | − | Reported | 2.20% | 0% | 2 | 2 |
| | | polycystic ovary syndrome | | (UUR) | | | | | (0%) | (0) | |
| 3277 | MT-TL1 | Poss. hypertension factor | G3277A | tRNA Leu | + | − | Reported | 2.90% | 0.1% | 32 | 1 |
| | | | | (UUR) | | | | | (0%) | (0) | |
| 3278 | MT-TL1 | Poss. hypertension factor | T3278C | tRNA Leu | + | − | Reported | 13.10% | 0% | 14 | 1 |
| | | | | (UUR) | | | | | (0%) | (0) | |
| 3280 | MT-TL1 | Myopathy | A3280G | tRNA Leu | − | + | Cfrm | Pathogenic | 0% | 0 | 6 |
| | | | | (UUR) | | | | | (0%) | (0) | |
| 3283 | MT-TL1 | Late onset ocular myopathy | G3283A | tRNA Leu | − | + | Reported | 58.70% | 0% | 0 | 1 |
| | | | | (UUR) | | | | | (0%) | (0) | |
| 3287 | MT-TL1 | Encephalomyopathy | C3287A | tRNA Leu | − | + | Reported | 38.30% | 0% | 0 | 2 |
| | | | | (UUR) | | | | | (0%) | (0) | |
| 3288 | MT-TL1 | Myopathy | A3288G | tRNA Leu | − | + | Reported | 36.10% | 0% | 0 | 3 |
| | | | | (UUR) | | | | | (0%) | (0) | |
| 3290 | MT-TL1 | Poss. hypertension factor | T3290C | tRNA Leu | + | − | Reported | 1.40% | 0.2% | 121 | 2 |
| | | | | (UUR) | | | | | (0%) | (0) | |
| 3291 | MT-TL1 | MELAS/Myopathy/ | T3291C | tRNA Leu | − | + | Cfrm | Pathogenic | 0% | 0 | 14 |
| | | Deafness + Cognitive | | (UUR) | | | | | (0%) | (0) | |
| | | Impairment | | | | | | | | | |
| 3302 | MT-TL1 | MM | A3302G | tRNA Leu | − | + | Cfrm | Pathogenic | 0% | 0 | 10 |
| | | | | (UUR) | | | | | (0%) | (0) | |
| 3303 | MT-TL1 | MMC | C3303T | tRNA Leu | + | + | Cfrm | Pathogenic | 0% | 0 | 12 |
| | | | | (UUR) | | | | | (0%) | (0) | |
| 4263 | MT-TI | Maternally inherited essential | A4263G | tRNA Ile | + | − | Reported | 67.80% | 0% | 4 | 4 |
| | | hypertension | | | | | | | (0%) | (0) | |
| 4267 | MT-TI | MM/CPEO | A4267G | tRNA Ile | − | + | Reported | 71.10% | 0% | 0 | 4 |
| | | | | | | | | | (0%) | (0) | |
| 4269 | MT-TI | FICP | A4269G | tRNA Ile | − | + | Reported | 82.80% | 0% | 0 | 9 |
| | | | | | | | | | (0%) | (0) | |
| 4274 | MT-TI | CPEO/Motor Neuron | T4274C | tRNA Ile | − | + | Reported | 85.50% | 0% | 0 | 5 |
| | | Disease | | | | | | | (0%) | (0) | |
| 4277 | MT-TI | HCM/Poss. hypertension | T4277C | tRNA Ile | + | − | Reported | 8.90% | 0% | 18 | 2 |
| | | factor | | | | | | | (0%) | (0) | |
| 4279 | MT-TI | Myoclonic epilepsy | A4279G | tRNA Ile | − | + | Reported | 54.90% | 0% | 0 | 1 |
| | | | | | | | | | (0%) | (0) | |
| 4281 | MT-TI | Recurrent Myoglobinuria | A4281G | tRNA Ile | − | + | Reported | 87.90% | 0% | 1 | 1 |
| | | | | | | | | | (0%) | (0) | |
| 4282 | MT-TI | CPEO Plus | G4282A | tRNA Ile | − | + | Reported | 82.30% | 0% | 0 | 1 |
| | | | | | | | | | (0%) | (0) | |
| 4284 | MT-TI | Varied familial presentation/ | G4284A | tRNA Ile | − | + | Reported | 35.30% | 0% | 2 | 6 |
| | | spastic paraparesis | | | | | | | (0%) | (0) | |
| 4285 | MT-TI | CPEO | T4285C | tRNA Ile | − | + | Reported | 84.%80 | 0% | 0 | 5 |
| | | | | | | | | | (0%) | (0) | |
| 4289 | MT-TI | Retinopathy + diabetes + | T4289C | tRNA Ile | − | + | Reported | 84.30% | 0% | 0 | 1 |
| | | dysphagia + cerebral atrophy | | | | | | | (0%) | (0) | |
| 4290 | MT-TI | Progressive Encephalopathy/ | T4290C | tRNA Ile | + | + | Reported | 47.70% | 0% | 0 | 4 |
| | | PEO, myopathy | | | | | | | (0%) | (0) | |
| 4291 | MT-TI | Hypomagnesemic Metabolic | T4291C | tRNA Ile | + | − | Reported | 31.80% | 0% | 0 | 1 |
| | | Syndrome | | | | | | | (0%) | (0) | |
| 4295 | MT-TI | MHCM/Maternally inherited | A4295G | tRNA Ile | + | + | Reported | 44.00% | 0.2% | 95 | 11 |
| | | hypertension/Maternally | | | | | | | (0%) | (0) | |
| | | inherited deafness | | | | | | | | | |
| 4296 | MT-TI | Leigh Syndrome | G4296A | tRNA Ile | − | + | Reported | 46.60% | 0% | 0 | 3 |
| | | | | | | | | | (0%) | (0) | |
| 4298 | MT-TI | CPEO/MS | G4298A | tRNA Ile | − | + | Cfrm | Pathogenic | 0% | 0 | 9 |
| | | | | | | | | | (0%) | (0) | |
| 4300 | MT-TI | MICM | A4300G | tRNA Ile | + | + | Cfrm | Pathogenic | 0% | 0 | 9 |
| | | | | | | | | | (0%) | (0) | |
| 4302 | MT-TI | CPEO | A4302G | tRNA Ile | − | + | Reported | 42.00% | 0% | 0 | 1 |
| | | | | | | | | | (0%) | (0) | |
| 4308 | MT-TI | CPEO | G4308A | tRNA Ile | − | + | Cfrm | Pathogenic | 0% | 0 | 2 |
| | | | | | | | | | (0%) | (0) | |
| 4309 | MT-TI | CPEO | G4309A | tRNA Ile | − | + | Reported | 64.10% | 0% | 1 | 3 |
| | | | | | | | | | (0%) | (0) | |
| 4314 | MT-TI | Poss. hypertension factor | T4314C | tRNA Ile | + | − | Reported | 1.70% | 0.1% | 42 | 1 |
| | | | | | | | | | (0%) | (0) | |
| 4316 | MT-TI | HCM with hearing loss/ | A4316G | tRNA Ile | + | + | Reported | 37.10% | 0.1% | 35 | 2 |
| | | poss. hypertension factor | | | | | | | (0%) | (0) | |
| 4317 | MT-TI | FICP/poss. Hypertension/ | A4317G | tRNA Ile | + | − | Reported | 2.10% | 0.1% | 38 | 11 |
| | | DEAF factor | | | | | | | (0%) | (0) | |
| 4317 | MT-TI | Ptosis, deafness, stroke-like | A4317del | tRNA Ile | − | − | Reported | 2.10% | 0% | 0 | 1 |
| | | episodes | | | | | | | (0%) | (0) | |
| 4320 | MT-TI | Mitochondrial | C4320T | tRNA Ile | − | + | Reported | 25.60% | 0% | 4 | 4 |
| | | Encephalocardiomyopathy | | | | | | | (0%) | (0) | |
| 4322 | MT-TI | Idiopathic Dilated | C4322CC | tRNA Ile | − | + | Reported | — | 0% | 3 | 1 |
| | | Cardiomopathy | | | | | | | (0%) | (0) | |
| 4322 | MT-TI | mtDNA deletion and | C4322del | tRNA Ile | + | − | Reported | 88.10% | 0% | 0 | 1 |
| | | depletion with dilated | | | | | | | (0%) | (0) | |
| | | cardiomyopathy | | | | | | | | | |
| 4332 | MT-TO | Encephalopathy/MELAS | G4332A | tRNA Gln | − | + | Cfrm | Pathogenic | 0% | 0 | 4 |
| | | | | | | | | | (0%) | (0) | |
| 4336 | MT-TO | ADPD/Hearing Loss & | T4336C | tRNA Gln | + | + | Unclear | 37.30% | 0.8% | 410 | 26 |
| | | Migraine/autism spectrum/ | | | | | | | (0%) | (0) | |
| | | intellectual disability | | | | | | | | | |
| 4343 | MT-TO | Poss. hypertension factor | A4343G | tRNA Gln | + | − | Reported | 5.10% | 0.1% | 53 | 1 |
| | | | | | | | | | (0%) | (0) | |
| 4345 | MT-TO | Poss. hypertension factor | C4345T | tRNA Gln | + | − | Reported | 13.20% | 0% | 2 | 1 |
| | | | | | | | | | (0%) | (0) | |
| 4353 | MT-TO | Poss. hypertension factor | T4353C | tRNA Gln | + | − | Reported | 31.60% | 0% | 23 | 1 |
| | | | | | | | | | (0%) | (0) | |
| 4363 | MT-TO | Metabolic syndrome and | T4363C | tRNA Gln | + | − | Reported | 9.56% | 0.1% | 45 | 5 |
| | | polycystic ovary syndrome/ | | | | | | | (0%) | (0) | |
| | | possibly associated w DEAF + | | | | | | | | | |
| | | RP + dev delay/ | | | | | | | | | |
| | | hypertension | | | | | | | | | |
| 4369 | MT-TO | Myopathy | A4369AA | tRNA Gln | − | + | Reported | — | 0% | 0 | 2 |
| | | | | | | | | | (0%) | (0) | |
| 4372 | MT-TO | Suspected mito disease | C4372T | tRNA Gln | − | + | Reported | 71.30% | 0% | 0 | 1 |
| | | | | | | | | | (0%) | (0) | |
| 4373 | MT-TO | Possibly LVNC-associated | T4373C | tRNA Gln | + | − | Reported | 79.10% | 0% | 8 | 1 |
| | | | | | | | | | (0%) | (0) | |
| 4381 | MT-TO | LHON | A4381G | tRNA Gln | + | − | Reported | 15.30% | 0% | 4 | 1 |
| | | | | | | | | | (0%) | (0) | |
| 4386 | MT-TO | Heart disease/myopathy/ | T4386C | tRNA Gln | + | − | Conflicting | 6.90% | 0.3% | 167 | 3 |
| | | hypertension | | | | | reports | | (0%) | (0) | |
| 4387 | MT-TO | Poss. hypertension factor | C4387A | tRNA Gln | + | − | Reported | 12.80% | 0% | 0 | 1 |
| | | | | | | | | | (0%) | (0) | |
| 4388 | MT-TO | Poss. hypertension factor, | A4388G | tRNA Gln | + | − | Reported | 0.10% | 0.1% | 64 | 2 |
| | | intellectual disability | | | | | | | (0%) | (0) | |
| 4392 | MT-TO | Poss. hypertension factor | C4392T | tRNA Gln | + | − | Reported | 15.70% | 0% | 18 | 1 |
| | | | | | | | | | (0%) | (0) | |
| 4395 | MT-TO | Poss. hypertension factor | A4395G | tRNA Gln | + | − | Reported | 0.20% | 0% | 24 | 1 |
| | | | | | | | | | (0%) | (0) | |
| 4401 | MT-NC2 | Hypertension + Ventricular | A4401G | NC2 Gln-Met | + | − | Reported | N/A | 0% | 3 | 3 |
| | | Hypertrophy | | spacer | | | | | (0%) | (0) | |
| 4403 | MT-TM | Mitochondrial myopathy | G4403A | tRNA Met | − | + | Reported | 84.80% | 0% | 0 | 1 |
| | | | | | | | | | (0%) | (0) | |
| 4409 | MT-TM | Mitochondrial myopathy | T4409C | tRNA Met | − | + | Reported | 46.50% | 0% | 0 | 5 |
| | | | | | | | | | (0%) | (0) | |
| 4410 | MT-TM | Poss. hypertension factor | C4410A | tRNA Met | + | − | Reported | 32.90% | 0% | 0 | 1 |
| | | | | | | | | | (0%) | (0) | |
| 4412 | MT-TM | Seizures with myopathy & | G4412A | tRNA Met | − | + | Reported | 76.50% | 0% | 0 | 1 |
| | | retinopathy | | | | | | | (0%) | (0) | |
| 4415 | MT-TM | EXIT & APS2 | A4415G | tRNA Met | − | + | Reported | 44.10% | 0% | 0 | 1 |
| | | | | | | | | | (0%) | (0) | |
| 4435 | MT-TM | LHON modulator/ | A4435G | tRNA Met | + | − | Reported | 13.80% | 0.1% | 52 | 2 |
| | | hypertension; autism | | | | | | | (0%) | (0) | |
| | | spectrum; intellectual | | | | | | | | | |
| | | disability | | | | | | | | | |
| 4437 | MT-TM | Hypotonia, seizure, muscle | C4437T | tRNA Met | + | − | Reported | 67.20% | 0% | 1 | 2 |
| | | weakness, lactic acidosis, | | | | | | | (0%) | (0) | |
| | | hearing loss | | | | | | | | | |
| 4440 | MT-TM | Mitochondrial myopathy | G4440A | tRNA Met | − | + | Reported | 58.20% | 0% | 0 | 3 |
| | | | | | | | | | (0%) | (0) | |
| 4450 | MT-TM | Myopathy/MELAS/Leigh | G4450A | tRNA Met | − | + | Cfrm | Pathogenic | 0% | 0 | 4 |
| | | Syndrome | | | | | | | (0%) | (0) | |
| 4456 | MT-TM | Poss. hypertension factor | C4456T | tRNA Met | − | + | Reported | 32.00% | 0% | 7 | 1 |
| | | | | | | | | | (0%) | (0) | |
| 4467 | MT-TM | Maternally inherited | C4467A | tRNA Met | − | + | Reported | 75.60% | 0% | 0 | 1 |
| | | hypertension | | | | | | | (0%) | (0) | |
| 5512 | MT-TW | Maternally inherited | A5512G | tRNA Trp | + | − | Reported | 38.60% | 0% | 8 | 1 |
| | | hypertension | | | | | | | (0%) | (0) | |
| 5513 | MT-TW | Mitochondrial | G5513A | tRNA Trp | − | + | Reported | 32.60% | 0% | 1 | 1 |
| | | encephalomyopathy with RP | | | | | | | (0%) | (0) | |
| 5514 | MT-TW | Neonatal onset mito disease | A5514G | tRNA Trp | + | − | Reported | 19.70% | 0.1% | 42 | 1 |
| | | | | | | | | | (0%) | (0) | |
| 5521 | MT-TW | Mitochondrial myopathy | G5521A | tRNA Trp | − | + | Cfrm | Pathogenic | 0% | 0 | 5 |
| | | | | | | | | | (0%) | (0) | |
| 5522 | MT-TW | Mitochondrial myopathy | G5522A | tRNA Trp | − | + | Reported | 83.00% | 0% | 0 | 2 |
| | | | | | | | | | (0%) | (0) | |
| 5523 | MT-TW | Leigh Syndrome | T5523G | tRNA Trp | − | + | Reported | 80.90% | 0% | 0 | 1 |
| | | | | | | | | | (0%) | (0) | |
| 5532 | MT-TW | Gastrointestinal Syndrome | G5532A | tRNA Trp | − | + | Reported | 19.40% | 0% | 1 | 3 |
| | | | | | | | | | (0%) | (0) | |
| 5537 | MT-TW | Leigh Syndrome | A5537insT | tRNA Trp | − | + | Cfrm | — | 0% | 0 | 5 |
| | | | | | | | | | (0%) | (0) | |
| 5538 | MT-TW | Encophalomyopathy | G5538A | tRNA Trp | − | + | Reported | 76.70% | 0% | 0 | 1 |
| | | | | | | | | | (0%) | (0) | |
| 5540 | MT-TW | Encephalomyopathy/DEAF | G5540A | tRNA Trp | − | + | Reported | 73.70% | 0% | 0 | 3 |
| | | | | | | | | | (0%) | (0) | |
| 5541 | MT-TW | MELAS + stroke-like episodes | C5541T | tRNA Trp | − | + | Reported | 84.30% | 0% | 0 | 1 |
| | | and cortical blindness + MRI | | | | | | | (0%) | (0) | |
| | | shows occipital lobe infarct | | | | | | | | | |
| 5543 | MT-TW | Mitochondrial myopathy | T5543C | tRNA Trp | − | + | Reported | 47.30% | 0% | 0 | 5 |
| | | | | | | | | | (0%) | (0) | |
| 5545 | MT-TW | HCM severe multisystem | C5545T | tRNA Trp | − | + | Reported | 53.00% | 0% | 0 | 1 |
| | | disorder | | | | | | | (0%) | (0) | |
| 5549 | MT-TW | DEMCHO | G5549A | tRNA Trp | − | + | Reported | 83.30% | 0% | 0 | 1 |
| | | | | | | | | | (0%) | (0) | |
| 5556 | MT-TW | Mito encephalomyopathy | G5556C | tRNA Trp | − | + | Reported | 44.50% | 0% | 0 | 1 |
| | | | | | | | | | (0%) | (0) | |
| 5556 | MT-TW | Combined OXPHOS defects | G5556A | tRNA Trp | − | + | Reported | 44.50% | 0% | 0 | 2 |
| | | | | | | | | | (0%) | (0) | |
| 5559 | MT-TW | Leigh Syndrome | A5559G | tRNA Trp | − | + | Reported | 70.10% | 0% | 0 | 1 |
| | | | | | | | | | (0%) | (0) | |
| 5567 | MT-TW | Myopathy | T5567C | tRNA Trp | − | + | Reported | 32.70% | 0.1% | 50 | 2 |
| | | | | | | | | | (0%) | (0) | |
| 5568 | MT-TW | DEAF | A5568G | tRNA Trp | + | − | Reported | 9.70% | 0% | 9 | 1 |
| | | | | | | | | | (0%) | (0) | |
| 5587 | MT-TA | LHON/possible DEAF | T5587C | tRNA Ala | + | + | Reported | 12.10% | 0.1% | 34 | 4 |
| | | modifier/dilated | | | | | | | (0%) | (0) | |
| | | cardiomyopathy/ | | | | | | | | | |
| | | hypertension | | | | | | | | | |
| 5591 | MT-TA | Myopathy | G5591A | tRNA Ala | − | + | Reported | 68.40% | 0% | 0 | 3 |
| | | | | | | | | | (0%) | (0) | |
| 5592 | MT-TA | Coronary Heart Disease | A5592G | tRNA Ala | + | − | Reported | 0.1% | 0.1% | 27 | 2 |
| | | | | | | | | | (0%) | (0) | |
| 5610 | MT-TA | Myopathy | G5610A | tRNA Ala | − | + | Reported | 38.70% | 0% | 0 | 1 |
| | | | | | | | | | (0%) | (0) | |
| 5613 | MT-TA | CPEO | T5613C | tRNA Ala | − | + | Reported | 59.30% | 0% | 0 | 1 |
| | | | | | | | | | (0%) | (0) | |
| 5628 | MT-TA | CPEO/DEAF enhancer/ | T5628C | tRNA Ala | − | + | Reported | 78.90% | 0.2% | 97 | 4 |
| | | gout | | | | | | | (0%) | (0) | |
| 5631 | MT-TA | Myopathy | G5631A | tRNA Ala | − | + | Reported | 43.40% | 0% | 1 | 2 |
| | | | | | | | | | (0%) | (0) | |
| 5636 | MT-TA | PEO | T5636C | tRNA Ala | − | + | Reported | 73.50% | 0% | 0 | 1 |
| | | | | | | | | | (0%) | (0) | |
| 5650 | MT-TA | Myopathy | G5650A | tRNA Ala | − | + | Cfrm | Pathogenic | 0% | 1 | 7 |
| | | | | | | | | | (0%) | (0) | |
| 5652 | MT-TA | Dilated Cardiomyopathy | C5652G | tRNA Ala | + | − | Reported | 69.90% | 0% | 0 | 1 |
| | | | | | | | | | (0%) | (0) | |
| 5655 | MT-TA | DEAF enhancer/ | T5655C | tRNA Ala | + | − | Reported | 26.70% | 0.6% | 324 | 3 |
| | | Hypertension risk | | | | | | | (0%) | (0) | |
| 5658 | MT-TA | Mitochondrial myopathy | T5658C | tRNA Asn | − | + | Reported | 94.30% | 0% | 0 | 1 |
| | | | | | | | | | (0%) | (0) | |
| 5667 | MT-TA | Ptosis | G5667A | tRNA Asn | − | − | Reported | 44.60% | 0% | 0 | 1 |
| | | | | | | | | | (0%) | (0) | |
| 5690 | MT-TN | CPEO + ptosis + proximal | A5690G | tRNA Asn | − | + | Cfrm | Pathogenic | 0% | 0 | 2 |
| | | myopathy | | | | | | | (0%) | (0) | |
| 5692 | MT-TN | CPEO/MM | T5692C | tRNA Asn | − | + | Reported | 46.60% | 0% | 0 | 4 |
| | | | | | | | | | (0%) | (0) | |
| 5693 | MT-TN | Encephalomyopathy | T5693C | tRNA Asn | + | − | Reported | 31.20% | 0% | 0 | 1 |
| | | | | | | | | | (0%) | (0) | |
| 5698 | MT-TN | CPEO/MM | G5698A | tRNA Asn | − | + | Reported | 47.70% | 0% | 1 | 4 |
| | | | | | | | | | (0%) | (0) | |
| 5703 | MT-TN | CPEO/MM | G5703A | tRNA Asn | − | + | Cfrm | Pathogenic | 0% | 0 | 2 |
| | | | | | | | | | (0%) | (0) | |
| 5709 | MT-TN | Ophthalmoparesis + | T5709C | tRNA Asn | − | + | Reported | 49.80% | 0% | 0 | 1 |
| | | respiratory impairment | | | | | | | (0%) | (0) | |
| 5728 | MT-TN | Multiorgan failure/myopathy | T5728C | tRNA Asn | − | + | Cfrm | Pathogenic | 0% | 1 | 3 |
| | | | | | | | | | (0%) | (0) | |
| 5780 | MT-TC | SNHL | G5780A | tRNA Cys | − | + | Reported | 35.50% | 0% | 15 | 1 |
| | | | | | | | | | (0%) | (0) | |
| 5783 | MT-TC | Myopathy/deafness/gout | G5783A | tRNA Cys | − | + | Reported | 66.90% | 0.1% | 43 | 2 |
| | | | | | | | | | (0%) | (0) | |
| 5802 | MT-TC | DEAF1555 increased | T5802C | tRNA Cys | + | − | Reported | 58.90% | 0% | 1 | 2 |
| | | penetrance | | | | | | | (0%) | (0) | |
| 5814 | MT-TC | Encephalopathy/gout | T5814C | tRNA Cys | − | + | L2b marker | 38.80% | 0.3% | 146 | 10 |
| | | | | | | | | | (0%) | (0) | |
| 5816 | MT-TC | Progressive Dystonia | A5816G | tRNA Cys | + | − | Reported | 59.00% | 0% | 0 | 3 |
| | | | | | | | | | (0%) | (0) | |
| 5821 | MT-TC | DEAF helper mut. | G5821A | tRNA Cys | + | − | Reported | 20.90% | 0.7% | 341 | 4 |
| | | | | | | | | | (0%) | (0) | |
| 5843 | MT-TY | FSGS/Mitochondrial | A5843G | tRNA Tyr | + | − | Reported | 8.40% | 0.4% | 207 | 1 |
| | | Cytopathy | | | | | | | (0%) | (0) | |
| 5874 | MT-TY | EXIT | T5874G | tRNA Tyr | − | + | Reported | 38.90% | 0% | 0 | 1 |
| | | | | | | | | | (0%) | (0) | |
| 7445 | MT-TS1 | DEAF | A7445C | tRNA Ser | + | − | Reported | — | 0% | 13 | 5 |
| | precursor | | | (UCN) | | | | | (0%) | (0) | |
| | 1 | | | precursor | | | | | | | |
| 7445 | MT-TS1 | SNHL | A7445G | tRNA Ser | + | + | Cfrm | — | 0% | 1 | 32 |
| | precursor | | | (UCN) | | | | | (0%) | (0) | |
| | 1 | | | precursor | | | | | | | |
| 7445 | MT-TS1 | SNHL | A7445T | tRNA Ser | + | − | Reported | | 0% | 3 | 1 |
| | precursor | | | (UCN) | | | | | (0%) | (0) | |
| | 1 | | | precursor | | | | | | | |
| 7451 | MT-TS1 | CPEO + ptosis | A7451T | tRNA Ser | − | + | Reported | 80.70% | 0% | 0 | 1 |
| | | | | (UCN) | | | | | (0%) | (0) | |
| | | | | precursor | | | | | | | |
| 7453 | MT-TS1 | Fatal neonatal lactic acidosis | G7453A | tRNA Ser | + | − | Reported | 68.00% | 0% | 0 | 2 |
| | | | | (UCN) | | | | | (0%) | (0) | |
| 7456 | MT-TS1 | | A7456G | tRNA Ser | + | − | Unclear | 16.00% | 0% | 1 | 1 |
| | | | | (UCN) | | | | | (0%) | (0) | |
| 7458 | MT-TS1 | | G7458A | tRNA Ser | − | + | Reported | 86.00% | 0% | 0 | 1 |
| | | | | (UCN) | | | | | (0%) | (0) | |
| 7462 | MT-TS1 | | C7462T | tRNA Ser | + | − | Reported | 11.20% | 0% | 6 | 1 |
| | | | | (UCN) | | | | | (0%) | (0) | |
| 7471 | MT-TS1 | PEM/AMDF/Motor neuron | C7471CC | tRNA Ser | + | + | Cfrm | — | 0% | 2 | 28 |
| | | disease-like | | (UCN) | | | | | (0%) | (0) | |
| 7472 | MT-TS1 | PEM/AMDF/Motor neuron | A7472CA | tRNA Ser | + | + | See | — | 0% | 0 | 1 |
| | | disease-like | | (UCN) | | | 7471insC | | (0%) | (0) | |
| 7472 | MT-TS1 | MM/DMDF modulator | A7472C | tRNA Ser | + | − | Reported | 3.20% | 0% | 9 | 3 |
| | | | | (UCN) | | | | | (0%) | (0) | |
| 7480 | MT-TS1 | MM | T7480G | tRNA Ser | − | + | Reported | 46.60% | 0% | 0 | 3 |
| | | | | (UCN) | | | | | (0%) | (0) | |
| 7486 | MT-TS1 | CPEO | G7486A | tRNA Ser | − | + | Reported | 50.50% | 0% | 0 | 1 |
| | | | | (UCN) | | | | | (0%) | (0) | |
| 7492 | MT-TS1 | Hypertension | C7492T | tRNA Ser | + | − | Reported | 0.10% | 0% | 8 | 1 |
| | | | | (UCN) | | | | | (0%) | (0) | |
| 7497 | MT-TS1 | MM/EXIT | G7497A | tRNA Ser | + | + | Cfrm | Pathogenic | 0% | 1 | 7 |
| | | | | (UCN) | | | | | (0%) | (0) | |
| 7501 | MT-TS1 | Cardiovascular disease; renal | T7501A | tRNA Ser | − | − | Reported | 1.90% | 0% | 1 | 3 |
| | | disease patient | | (UCN) | | | | | (0%) | (0) | |
| 7505 | MT-TS1 | Maternally inherited hearing | T7505C | tRNA Ser | + | − | Reported | 58.60% | 0% | 0 | 2 |
| | | loss | | (UCN) | | | | | (0%) | (0) | |
| 7506 | MT-TS1 | PEO with hearing loss | G7506A | tRNA Ser | − | + | Reported | 81.40% | 0% | 0 | 1 |
| | | | | (UCN) | | | | | (0%) | (0) | |
| 7510 | MT-TS1 | SNHL | T7510C | tRNA Ser | − | + | Cfrm | Pathogenic | 0% | 1 | 13 |
| | | | | (UCN) | | | | | (0%) | (0) | |
| 7511 | MT-TS1 | SNHL/Deafness | T7511C | tRNA Ser | + | + | Cfrm | Pathogenic | 0% | 2 | 20 |
| | | | | (UCN) | | | | | (0%) | (0) | |
| 7512 | MT-TS1 | PEM/MERME | T7512C | tRNA Ser | + | + | Reported | 64.20% | 0% | 0 | 10 |
| | | | | (UCN) | | | | | (0%) | (0) | |
| 7520 | MT-TD | Sporadic bilateral optic | G7520A | tRNA Asp | − | − | Reported | 54.90% | 0% | 0 | 1 |
| | | neuropathy | | | | | | | (0%) | (0) | |
| 7526 | MT-TD | Mitochondrial myopathy | A7526G | tRNA Asp | − | + | Reported | 50.40% | 0% | 0 | 1 |
| | | | | | | | | | (0%) | (0) | |
| 7539 | MT-TD | Multisystemic mitochondrial | C7539T | tRNA Asp | − | + | Reported | 93.70% | 0% | 0 | 1 |
| | | disorder | | | | | | | (0%) | (0) | |
| 7543 | MT-TD | MEPR | A7543G | tRNA Asp | − | + | Reported | 67.30% | 0.1% | 47 | 1 |
| | | | | | | | | | (0%) | (0) | |
| 7551 | MT-TD | DEAF increased penetrance | A7551G | tRNA Asp | + | − | Reported | 28.90% | 0% | 2 | 2 |
| | | (1555G helper) | | | | | | | (0%) | (0) | |
| 7554 | MT-TD | Myopathy + ataxia + | G7554A | tRNA Asp | − | + | Reported | 71.20% | 0% | 1 | 1 |
| | | nystagmus + | | | | | | | (0%) | (0) | |
| | | migraines + lactic acidosis | | | | | | | | | |
| 8296 | MT-TK | DMDF/MERRF/HCM/ | A8296G | tRNA Lys | + | + | Reported | 72.30% | 0.1% | 37 | 17 |
| | | epilepsy | | | | | | | (0%) | (0) | |
| 8299 | MT-TK | PEO + respiratory impairment | G8299A | tRNA Lys | − | + | Reported | 63.80% | 0% | 0 | 1 |
| | | | | | | | | | (0%) | (0) | |
| 8302 | MT-TK | Encephalopathy | A8302T | tRNA Lys | + | − | Unclear | 15.20% | 0% | 0 | 1 |
| | | | | | | | | | (0%) | (0) | |
| 8304 | MT-TK | Epilepsy + ataxia + visual | G8304A | tRNA Lys | − | + | Reported | 89.70% | 0% | 0 | 1 |
| | | disturbance + deafness | | | | | | | (0%) | (0) | |
| 8305 | MT-TK | Mitochondrial myopathy | C8305T | tRNA Lys | − | + | Reported | 74.50% | 0% | 0 | 3 |
| | | | | | | | | | (0%) | (0) | |
| 8306 | MT-TK | Severe adult-onset | T8306C | tRNA Lys | − | + | Cfrm | Pathogenic | 0% | 0 | 3 |
| | | multisymptom myopathy/ | | | | | | | (0%) | (0) | |
| | | Myoclonic epilepsy | | | | | | | | | |
| 8311 | MT-TK | Poss. hypertension factor | T8311C | tRNA Lys | + | − | Reported | 6.80% | 0.1% | 56 | 1 |
| | | | | | | | | | (0%) | (0) | |
| 8313 | MT-TK | MNGIE/Progressive mito | G8313A | tRNA Lys | − | + | Cfrm | Pathogenic | 0% | 1 | 6 |
| | | cytopathy | | | | | | | (0%) | (0) | |
| 8316 | MT-TK | MELAS | T8316C | tRNA Lys | − | + | Reported | 80.20% | 0% | 0 | 3 |
| | | | | | | | | | (0%) | (0) | |
| 8319 | MT-TK | Kearns-Sayre syndrome | A8319G | tRNA Lys | − | + | Reported | 69.60% | 0% | 0 | 1 |
| | | | | | | | | | (0%) | (0) | |
| 8326 | MT-TK | Mitochondrial Cytopathy | A8326G | tRNA Lys | − | + | Reported | 46.20% | 0% | 0 | 3 |
| | | | | | | | | | (0%) | (0) | |
| 8328 | MT-TK | Mito Encephalopathy/EXIT | G8328A | tRNA Lys | − | + | Reported | 83.30% | 0% | 0 | 5 |
| | | with myopathy and ptosis | | | | | | | (0%) | (0) | |
| 8332 | MT-TK | Dystonia and stroke-like | A8332G | tRNA Lys | + | − | Reported | 62.80% | 0% | 0 | 1 |
| | | episodes | | | | | | | (0%) | (0) | |
| 8337 | MT-TK | Poss. hypertension factor | T8337C | tRNA Lys | + | − | Reported | 6.80% | 0.3% | 175 | 1 |
| | | | | | | | | | (0%) | (0) | |
| 8340 | MT-TK | Myopathy/Exercise | G8340A | tRNA Lys | − | + | Cfrm | Pathogenic | 0% | 0 | 7 |
| | | Intolerance/Eye | | | | | | | (0%) | (0) | |
| | | disease + SNHL | | | | | | | | | |
| 8342 | MT-TK | PEO and Myoclonus | G8342A | tRNA Lys | − | + | Reported | 77.20% | 0% | 0 | 4 |
| | | | | | | | | | (0%) | (0) | |
| 8343 | MT-TK | Metabolic syndrome and | A8343G | tRNA Lys | + | − | Reported | 4.70% | 0.1% | 53 | 3 |
| | | polycystic ovary syndrome/ | | | | | | | (0%) | (0) | |
| | | possible PD risk factor | | | | | | | | | |
| 8344 | MT-TK | MERRF; Other-LD/ | A8344G | tRNA Lys | − | + | Cfrm | Pathogenic | 0% | 4 | 124 |
| | | Depressive mood disorder/ | | | | | | | (0%) | (0) | |
| | | leukoencephalopathy/HiCM | | | | | | | | | |
| 8347 | MT-TK | Poss. hypertension factor | A8347G | tRNA Lys | + | − | Reported | 2.60% | 0% | 19 | 2 |
| | | | | | | | | | (0%) | (0) | |
| 8348 | MT-TK | Cardiomyopathy/SNHL/ | A8348G | tRNA Lys | + | + | Reported | 33.80% | 0.2% | 118 | 8 |
| | | poss. hypertension factor | | | | | | | (0%) | (0) | |
| 8355 | MT-TK | Myopathy | T8355C | tRNA Lys | − | + | Reported | 67.20% | 0% | 0 | 2 |
| | | | | | | | | | (0%) | (0) | |
| 8356 | MT-TK | MERRF | T8356C | tRNA Lys | − | + | Cfrm | Pathogenic | 0% | 0 | 10 |
| | | | | | | | | | (0%) | (0) | |
| 8357 | MT-TK | Multiple symmetric | T8357C | tRNA Lys | − | + | Reported | 59.10% | 0% | 0 | 1 |
| | | lipomatosis | | | | | | | (0%) | (0) | |
| 8361 | MT-TK | MERRF | G8361A | tRNA Lys | − | + | Reported | 64.80% | 0% | 0 | 3 |
| | | | | | | | | | (0%) | (0) | |
| 8362 | MT-TK | Myopathy | T8362G | tRNA Lys | − | + | Reported | 93.00% | 0% | 0 | 5 |
| | | | | | | | | | (0%) | (0) | |
| 8363 | MT-TK | MICM + DEAF/MERRF/ | G8363A | tRNA Lys | − | + | Cfrm | Pathogenic | 0% | 0 | 20 |
| | | Autism/LS/ | | | | | | | (0%) | (0) | |
| | | Ataxia + Lipomas | | | | | | | | | |
| 9997 | MT-TG | MHCM | T9997C | tRNA Gly | − | + | Reported | 80.30% | 0% | 1 | 5 |
| | | | | | | | | | (0%) | (0) | |
| 10003 | MT-TG | Hypertension | T10003C | tRNA Gly | − | − | Reported | 0.40% | 0% | 8 | 1 |
| | | | | | | | | | (0%) | (0) | |
| 10006 | MT-TG | CIPO/Encephalopathy | A10006G | tRNA Gly | + | − | Unclear | 19.30% | 0% | 9 | 4 |
| | | | | | | | | | (0%) | (0) | |
| 10010 | MT-TG | PEM | T10010C | tRNA Gly | − | + | Cfrm | Pathogenic | 0% | 0 | 2 |
| | | | | | | | | | (0%) | (0) | |
| 10014 | MT-TG | Myopathy | G10014A | tRNA Gly | + | − | Unclear | 60.90% | 0% | 1 | 1 |
| | | | | | | | | | (0%) | (0) | |
| 10044 | MT-TG | SIDS | A10044G | tRNA Gly | − | + | Unclear | 34.70% | 0.3% | 135 | 8 |
| | | | | | | | | | (0%) | (0) | |
| 10406 | MT-TR | Mitochondrial myopathy | G10406A | tRNA Arg | − | + | Reported | 72.30% | 0% | 0 | 2 |
| | | | | | | | | | (0%) | (0) | |
| 10411 | MT-TR | Dilated Cardiomyopathy | A10411T | tRNA Arg | + | − | Reported | 26.40% | 0% | 0 | 1 |
| | | | | | | | | | (0%) | (0) | |
| 10415 | MT-TR | Dilated Cardiomyopathy | T10415C | tRNA Arg | + | − | Reported | 76.50% | 0% | 0 | 1 |
| | | | | | | | | | (0%) | (0) | |
| 10437 | MT-TR | Mitochondrial myopathy | G10437A | tRNA Arg | − | + | Reported | 51.70% | 0% | 0 | 1 |
| | | | | | | | | | (0%) | (0) | |
| 10438 | MT-TR | Progressive Encephalopathy | A10438G | tRNA Arg | − | + | Reported | 46.20% | 0% | 0 | 1 |
| | | | | | | | | | (0%) | (0) | |
| 10450 | MT-TR | Combined OXPHOS defects | A10450G | tRNA Arg | − | + | Reported | 69.60% | 0% | 0 | 1 |
| | | & severe multisystem | | | | | | | (0%) | (0) | |
| | | disorder | | | | | | | | | |
| 10454 | MT-TR | DEAF helper mut. | T10454C | tRNA Arg | + | − | Reported | 4.80% | 0.4% | 181 | 3 |
| | | | | | | | | | (0%) | (0) | |
| 12146 | MT-TH | MELAS | A12146G | tRNA His | + | + | Reported | 61.60% | 0% | 0 | 1 |
| | | | | | | | | | (0%) | (0) | |
| 12147 | MT-TH | MERRF-MELAS/ | G12147A | tRNA His | − | + | Cfrm | Pathogenic | 0% | 0 | 1 |
| | | Encephalopathy | | | | | | | (0%) | (0) | |
| 12148 | MT-TH | Developmental delay, optic | T12148C | tRNA His | − | + | Reported | 74.70% | 0% | 1 | 1 |
| | | atrophy, cataract, hearing | | | | | | | (0%) | (0) | |
| | | loss, myopathy | | | | | | | | | |
| 12183 | MT-TH | RP + DEAF | G12183A | tRNA His | − | + | Reported | 70.30% | 0% | 1 | 1 |
| | | | | | | | | | (0%) | (0%) | |
| 12187 | MT-TH | Asthenozoospermia | C12187A | tRNA His | + | − | Reported | 15.40% | 0% | 0 | 1 |
| | | | | | | | | | (0%) | (0%) | |
| 12192 | MT-TH | MICM | G12192A | tRNA His | + | − | Reported | 4.50% | 0.2% | 112 | 2 |
| | | | | | | | | | (0%) | (0) | |
| 12201 | MT-TH | Maternally inherited non- | T12201C | tRNA His | − | + | Reported | 66.70% | 0% | 1 | 1 |
| | | syndromic deafness | | | | | | | (0%) | (0) | |
| 12206 | MT-TH | MELAS-like | C12206T | tRNA His | − | + | Reported | 44.20% | 0% | 0 | 1 |
| | | encephalopathy + bilateral | | | | | | | (0%) | (0) | |
| | | optic atrophy | | | | | | | | | |
| 12207 | MT-TS2 | Myopathy/Encephalopathy | G12207A | tRNA Ser | − | + | Reported | 76.40% | 0% | 0 | 3 |
| | | | | (AGY) | | | | | (0%) | (0) | |
| 12224 | MT-TS2 | DEAF helper mut. | C12224T | tRNA Ser | + | − | Reported | 30.40% | 0% | 4 | 1 |
| | | | | (AGY) | | | | | (0%) | (0) | |
| 12236 | MT-TS2 | DEAF | G12236A | tRNA Ser | + | − | Reported | 2.20% | 0.7% | 373 | 4 |
| | | | | (AGY) | | | | | (0%) | (0) | |
| 12246 | MT-TS2 | CIPO | C12246A | tRNA Ser | − | − | Reported | 3.20% | 0% | 1 | 2 |
| | | | | (AGY) | | | | | (0%) | (0) | |
| 12258 | MT-TS2 | DMDF/RP + SNHL | C12258A | tRNA Ser | − | + | Cfrm | Pathogenic | 0% | 1 | 7 |
| | | | | (AGY) | | | | | (0%) | (0) | |
| 12261 | MT-TS2 | Myopathy + epilepsy + retinal | T12261C | tRNA Ser | − | + | Reported | 65.30% | 0% | 0 | 1 |
| | | degeneration + DEAF | | (AGY) | | | | | (0%) | (0) | |
| 12262 | MT-TS2 | Progressive | C12262A | tRNA Ser | − | + | Reported | 84.50% | 0% | 0 | 1 |
| | | MM + Deafness + Seizures | | (AGY) | | | | | (0%) | (0) | |
| 12264 | MT-TS2 | Multisystem Disease with | C12264T | tRNA Ser | + | + | Reported | 79.30% | 0% | 0 | 2 |
| | | Cataracts/ | | (AGY) | | | | | (0%) | (0) | |
| | | Myopathy + epilepsy + | | | | | | | | | |
| | | DEAF + atypical autism | | | | | | | | | |
| 12276 | MT-TL2 | CPEO | G12276A | tRNA Leu | − | + | Cfrm | Pathogenic | 0% | 1 | 3 |
| | | | | (CUN) | | | | | (0%) | (0) | |
| 12280 | MT-TL2 | Hypertension | A12280G | tRNA Leu | + | − | Reported | 6.50% | 0.1% | 72 | 1 |
| | | | | (CUN) | | | | | (0%) | (0) | |
| 12283 | MT-TL2 | CPEO | G12283A | tRNA Leu | − | + | Reported | 43.20% | 0% | 1 | 2 |
| | | | | (CUN) | | | | | (0%) | (0) | |
| 12293 | MT-TL2 | Axial mitochondrial | G12293A | tRNA Leu | − | + | Reported | 66.90% | 0% | 0 | 1 |
| | | myopathy | | (CUN) | | | | | (0%) | (0) | |
| 12294 | MT-TL2 | CPEO/ | G12294A | tRNA Leu | − | + | Cfrm | Pathogenic | 0% | 0 | 2 |
| | | EXIT + Ophthalmoplegia | | (CUN) | | | | | (0%) | (0) | |
| 12297 | MT-TL2 | Dilated Cardiomyopathy/LS/ | T12297C | tRNA Leu | + | + | Reported | 47.30% | 0.1% | 29 | 5 |
| | | Failure to Thrive & LA | | (CUN) | | | | | (0%) | (0) | |
| 12299 | MT-TL2 | MELAS | A12299C | tRNA Leu | − | + | Reported | 53.00% | 0% | 0 | 1 |
| | | | | (CUN) | | | | | (0%) | (0) | |
| 12300 | MT-TL2 | 3243 suppressor mutant | G12300A | tRNA Leu | − | + | Reported | 51.70% | 0% | 0 | 4 |
| | | | | (CUN) | | | | | (0%) | (0) | |
| 12308 | MT-TL2 | CPEO/Stroke/CM/Breast | A12308G | tRNA Leu | + | + | Reported | 42.00% | 12.4% | 6215 | 12 |
| | | & Renal & Prostate Cancer | | (CUN) | | | | | (0%) | (0) | |
| | | Risk/Altered brain pH/sCJD | | | | | | | | | |
| 12311 | MT-TL2 | CPEO | T12311C | tRNA Leu | + | + | Reported | 34.40% | 0.1% | 57 | 3 |
| | | | | (CUN) | | | | | (0%) | (0) | |
| 12313 | MT-TL2 | FSHD | T12313C | tRNA Leu | − | + | Reported | 73.20% | 0% | 0 | 1 |
| | | | | (CUN) | | | | | (0%) | (0) | |
| 12315 | MT-TL2 | CPEO/KSS/possible | G12315A | tRNA Leu | − | + | Cfrm | Pathogenic | 0% | 0 | 13 |
| | | carotid atherosclerosis risk, | | (CUN) | | | | | (0%) | (0) | |
| | | trend toward myocardial | | | | | | | | | |
| | | infarction risk | | | | | | | | | |
| 12316 | MT-TL2 | CPEO | G12316A | tRNA Leu | − | + | Cfrm | Pathogenic | 0% | 0 | 2 |
| | | | | (CUN) | | | | | (0%) | (0) | |
| 12317 | MT-TL2 | CPEO + ptosis + myopathy + | T12317C | tRNA Leu | − | + | Reported | 41.30% | 0% | 1 | 1 |
| | | exercise intolerance + diabetes | | (CUN) | | | | | (0%) | (0) | |
| 12320 | MT-TL2 | MM | A12320G | tRNA Leu | − | + | Reported | 37.30% | 0% | 0 | 7 |
| | | | | (CUN) | | | | | (0%) | (0) | |
| 14674 | MT-TE | Reversible COX deficiency | T14674C | tRNA Glu | + | − | Cfrm | Pathogenic | 0% | 7 | 6 |
| | | myopathy | | | | | | | (0%) | (0) | |
| 14674 | MT-TE | Reversible COX deficiency | T14674G | tRNA Glu | + | − | Reported | 29.46% | 0% | 0 | 1 |
| | | myopathy | | | | | | | (0%) | (0) | |
| 14680 | MT-TE | Mitochondrial | C14680A | tRNA Glu | − | + | Reported | 35.50% | 0% | 0 | 1 |
| | | encephalomyopathy | | | | | | | (0%) | (0) | |
| 14685 | MT-TE | Cataracts w spastic | G14685A | tRNA Glu | − | + | Reported | 77.40% | 0% | 0 | 1 |
| | | paraparesis & ataxia | | | | | | | (0%) | (0) | |
| 14687 | MT-TE | Mito myopathy w respiratory | A14687G | tRNA Glu | + | − | Reported | 7.00% | 0.6% | 299 | 3 |
| | | failure; intellectual disability | | | | | | | (0%) | (0) | |
| 14692 | MT-TE | LHON helper/Maternally | A14692G | tRNA Glu | + | − | Reported | 2.40% | 0% | 19 | 3 |
| | | inherited diabetes & deafness | | | | | | | (0%) | (0) | |
| 14693 | MT-TE | MELAS/LHON/DEAF/ | A14693G | tRNA Glu | + | + | Reported | 39.50% | 0.5% | 262 | 12 |
| | | hypertension helper | | | | | | | (0%) | (0) | |
| 14696 | MT-TE | Progressive Encephalopathy | A14696G | tRNA Glu | − | + | Reported | 22.00% | 0.1% | 46 | 1 |
| | | | | | | | | | (0%) | (0) | |
| 14709 | MT-TE | MM + DMDF/ | T14709C | tRNA Glu | + | + | Cfrm | Pathogenic | 0% | 1 | 22 |
| | | Encephalomyopathy/ | | | | | | | (0%) | (0) | |
| | | Dementia + diabetes + | | | | | | | | | |
| | | ophthalmoplegia | | | | | | | | | |
| 14710 | MT-TE | Encephalomyopathy + | G14710A | tRNA Glu | − | + | Cfrm | Pathogenic | 0% | 0 | 5 |
| | | Retinopathy | | | | | | | (0%) | (0) | |
| 14721 | MT-TE | Isolated complex I deficiency | G14721A | tRNA Glu | − | + | Reported | 82.50% | 0% | 0 | 1 |
| | | | | | | | | | (0%) | (0) | |
| 14723 | MT-TE | CPEO + Myopathy | T14723C | tRNA Glu | − | + | Reported | 23.50% | 0% | 0 | 2 |
| | | | | | | | | | (0%) | (0) | |
| 14724 | MT-TE | Mito Leukoencephalopathy | G14724A | tRNA Glu | − | + | Reported | 88.80% | 0% | 0 | 3 |
| | | | | | | | | | (0%) | (0) | |
| 14728 | MT-TE | Late-onset mitochondrial | T14728C | tRNA Glu | − | + | Reported | 48.50% | 0% | 0 | 1 |
| | | encephalomyopathy | | | | | | | (0%) | (0) | |
| 14739 | MT-TE | EXET | G14739A | tRNA Glu | − | + | Reported | 62.10% | 0% | 0 | 2 |
| | | | | | | | | | (0%) | (0) | |
| 15894 | MT-TT | Gout | G15894A | tRNA Thr | + | − | Reported | 28.20% | 0.1% | 29 | 1 |
| | | | | | | | | | (0%) | (0) | |
| 15908 | MT-TT | DEAF helper mut. | T15908C | tRNA Thr | + | − | Reported | 28.00% | 0.3% | 127 | 2 |
| | | | | | | | | | (0%) | (0) | |
| 15909 | MT-TT | Hypertesion | A15909G | tRNA Thr | + | − | Reported | 25.90% | 0% | 7 | 2 |
| | | | | | | | | | (0%) | (0) | |
| 15915 | MT-TT | Encephalomyopathy | G15915A | tRNA Thr | − | + | Reported | 73.70% | 0% | 1 | 2 |
| | | | | | | | | | (0%) | (0) | |
| 15923 | MT-TT | LIMM/MERRF/mito | A15923G | tRNA Thr | − | + | Reported | 46.00% | 0% | 0 | 5 |
| | | disease | | | | | | | (0%) | (0) | |
| 15924 | MT-TT | LIMM | A15924G | tRNA Thr | − | − | Reported | 22.70% | 3.5% | 1764 | 6 |
| | | | | | | | | | (0%) | (0) | |
| 15927 | MT-TT | LHON/Multiple Sclerosis/ | G15927A | tRNA Thr | + | − | Reported | 16.20% | 0.9% | 430 | 12 |
| | | DEAF 1555 increased | | | | | | | (0%) | (0) | |
| | | penetrance/CHD | | | | | | | | | |
| 15928 | MT-TT | Multiple Sclerosis/idiopathic | G15928A | tRNA Thr | + | − | Reported | 20.20% | 4.9% | 2447 | 7 |
| | | repeat miscarriage/AD | | | | | | | (0%) | (0) | |
| | | protection | | | | | | | | | |
| 15933 | MT-TT | Suspected mito disease | G15933A | tRNA Thr | + | − | Reported | 66.80% | 0% | 0 | 1 |
| | | | | | | | | | (0%) | (0) | |
| 15942 | MT-TT | Possibly LVNC-associated | T15942C | tRNA Thr | + | − | Reported | 28.60% | 0.8% | 408 | 1 |
| | | | | | | | | | (0%) | (0) | |
| 15944 | MT-TT | MM | T15944del | tRNA Thr | + | − | Conflicting | 19.90% | 1.5% | 754 | 2 |
| | | | | | | | reports | | (0%) | (0) | |
| 15950 | MT-TT | Dopaminergic nerve cell | G15950A | tRNA Thr | + | − | Reported | 54.50% | 0% | 1 | 1 |
| | | death (PD) | | | | | | | (0%) | (0) | |
| 15951 | MT-TT | LHON/LHON modulator | A15951G | tRNA Thr | + | − | Conflicting | 23.70% | 0.8% | 381 | 6 |
| | | | | | | | reports | | (0%) | (0) | |
| 15965 | MT-TP | Dopaminergic nerve cell | A15965G | tRNA Pro | + | − | Reported | 2.10% | 0% | 2 | 1 |
| | | death (PD) | | | | | | | (0%) | (0) | |
| 15967 | MT-TT | MERRF-like disease | G15967A | tRNA Pro | − | + | Reported | 78.90% | 0% | 0 | 2 |
| | | | | | | | | | (0%) | (0) | |
| 15975 | MT-TT | Ataxia + RP + deafness | C15975T | tRNA Pro | − | + | Reported | 78.30% | 0% | 0 | 1 |
| | | | | | | | | | (0%) | (0) | |
| 15990 | MT-TT | MM | C15990T | tRNA Pro | − | + | Reported | 51.70% | 0% | 0 | 4 |
| | | | | | | | | | (0%) | (0) | |
| 15995 | MT-TT | Mitochondrial cytopathy | G15995A | tRNA Pro | − | + | Reported | 80.00% | 0% | 0 | 2 |
| | | | | | | | | | (0%) | (0) | |
| 15998 | MT-TT | Mitochondrial myopathy | A15998T | tRNA Pro | − | + | Reported | 57.50% | 0% | 0 | 1 |
| | | | | | | | | | (0%) | (0) | |
| 16002 | MT-TT | Mitochondrial cytopathy | T16002C | tRNA Pro | − | + | Reported | 75.80% | 0% | 0 | 1 |
| | | | | | | | | | (0%) | (0) | |
| 16015 | MT-TT | Mitochondrial myopathy | T16015C | tRNA Pro | − | + | Reported | 50.40% | 0% | 0 | 1 |
| | | | | | | | | | (0%) | (0) | |
| 16018 | MT-TT | Dilated cardiomyopathy (15 | T16018TTCT | tRNA Pro | − | + | Reported | — | 0% | 0 | 1 |
| | | bp dup), alternate notation | CTGTTCTT | | | | | | (0%) | (0) | |
| | | | TCAT(SEQ | | | | | | | | |
| | | | ID NO: 4) | | | | | | | | |
| 16021 | MT-TT | Mitochondrial myopathy | 16021_16022 | tRNA Pro | − | + | Reported | — | 0% | 0 | 1 |
| | | | delCT | | | | | | (0%) | (0) | |
| 16023 | MT-TT | Migraine + pigmentary | G16023A | tRNA Pro | − | + | Reported | 83.70% | 0% | 0 | 1 |
| | | retinopathy + deafness + | | | | | | | (0%) | (0) | |
| | | leukariosis | | | | | | | | | |
| 16032 | MT-TT | Dilated cardiomyopathy (15 | T16032TTCT | tRNA Pro | − | + | Reported | — | 0% | 1 | 1 |
| | | bp dup) | CTGTTCTT | | | | | | (0%) | (0) | |
| | | | TCAT (SEQ | | | | | | | | |
| | | | ID NO: 4) | | | | | | | | |
| 16033 | MT-TP | Dilated cardiomyopathy (15 | G16033TCT | tRNA Pro | − | + | Reported | — | 0% | 0 | 1 |
| | | bp dup), alternate notation | CTGTTCTT | | | | | | (0%) | (0) | |
| | | | TCATG(SEQ | | | | | | | | |
| | | | ID NO: 5) | | | | | | | | |

Column Heading Key: A: Position; B: Locus; C: Disease; D: Allele; E: RNA; F: Homoplasmy; G: Heteroplasmy; H: Status; I: MitoTip; J: GB Freq FL (CR); K: GB Seqs FL (CR); L: Reference [symbol]
indicates data missing or illegible when filed

TABLE 2
MITOMAP: Mitochondrial DNA Base Substitution Diseases:
rRNA/tRNA Mutations with Cfrm Status

| A | B | C | D | E | F | G | H | I | J | K | L |
|---|---|---|---|---|---|---|---|---|---|---|---|
| 583 | MT-TF | MELAS/MM & | G583A | tRNA | − | + | Cfrm | Pathogenic | 0.0% | 0 (0) | 3 |
| | | EXIT | | Phe | | | | | (0.0%) | | |
| 616 | MT-TF | Maternally inherited | T616C | tRNA | + | + | Cfrm | Pathogenic | 0.0% | 1 (0) | 2 |
| | | epilepsy/kidney disease | | Phe | | | | | (0.0%) | | |
| 1494 | MT-RNR1 | DEAF | C1494T | 12S | + | − | Cfrm | N/A | 0.0% | 4 (0) | 29 |
| | | | | rRNA | | | | | (0.0%) | | |
| 1555 | MT-RNR1 | DEAF; autism | A1555G | 12S | + | − | Cfrm | N/A | 0.1% | 74 (0) | 138 |
| | | spectrum intellectual | | rRNA | | | | | (0.0%) | | |
| | | disability; possibly | | | | | | | | | |
| | | antiatherosclerotic | | | | | | | | | |
| 1606 | MT-TV | AMDF | G1606A | tRNA | − | + | Cfrm | Pathogenic | 0.0% | 0 (0) | 4 |
| | | | | Val | | | | | (0.0%) | | |
| 1630 | MT-TV | MNGIE-like disease/ | A1630G | tRNA | − | + | Cfrm | Pathogenic | 0.0% | 0 (0) | 1 |
| | | MELAS | | Val | | | | | (0.0%) | | |
| 1644 | MT-TV | LS/HCM/MELAS | G1644A | tRNA | − | + | Cfrm | Pathogenic | 0.0% | 0 (0) | 4 |
| | | | | Val | | | | | (0.0%) | | |
| 3243 | MT-TL1 | MELAS/LS/ | A3243G | tRNA | − | + | Cfrm | Pathogenic | 0.0% | 9 (0) | 392 |
| | | DMDF/MIDD/ | | Leu | | | | | (0.0%) | | |
| | | SNHL/CPEO/MM/ | | (UUR) | | | | | | | |
| | | FSGS/ASD/ | | | | | | | | | |
| | | Cardiac + multi-organ | | | | | | | | | |
| | | dysfunction | | | | | | | | | |
| 3243 | MT-TL1 | MM/MELAS/ | A3243T | tRNA | − | + | Cfrm | Pathogenic | 0.0% | 0 (0) | 6 |
| | | SNHL/CPEO | | Leu | | | | | (0.0%) | | |
| | | | | (UUR) | | | | | | | |
| 3256 | MT-TL1 | MELAS; possible | C3256T | tRNA | − | + | Cfrm | Pathogenic | 0.0% | 0 (0) | 18 |
| | | atherosclerosis risk | | Leu | | | | | (0.0%) | | |
| | | | | (UUR) | | | | | | | |
| 3258 | MT-TL1 | MELAS/Myopathy | T3258C | tRNA | − | + | Cfrm | Pathogenic | 0.0% | 1 (0) | 5 |
| | | | | Leu | | | | | (0.0%) | | |
| | | | | (UUR) | | | | | | | |
| 3260 | MT-TL1 | MMC/MELAS | A3260G | tRNA | − | + | Cfrm | Pathogenic | 0.0% | 0 (0) | 10 |
| | | | | Leu | | | | | (0.0%) | | |
| | | | | (UUR) | | | | | | | |
| 3271 | MT-TL1 | PEM/retinal | T3271d | tRNA | − | + | Cfrm | Pathogenic | 0.0% | 0 (0) | 3 |
| | | dystrophy in | el | Leu | | | | | (0.0%) | | |
| | | MELAS | | (UUR) | | | | | | | |
| 3271 | MT-TL1 | MELAS/DM | T3271C | tRNA | − | + | Cfrm | Pathogenic | 0.0% | 0 (0) | 25 |
| | | | | Leu | | | | | (0.0%) | | |
| | | | | (UUR) | | | | | | | |
| 3280 | MT-TL1 | Myopathy | A3280G | tRNA | − | + | Cfrm | Pathogenic | 0.0% | 0 (0) | 5 |
| | | | | Leu | | | | | (0.0%) | | |
| | | | | (UUR) | | | | | | | |
| 3291 | MT-TL1 | MELAS/Myopathy/ | T3291C | tRNA | − | + | Cfrm | Pathogenic | 0.0% | 0 (0) | 14 |
| | | Deafness + Cognitive | | Leu | | | | | (0.0%) | | |
| | | Impairment | | (UUR) | | | | | | | |
| 3302 | MT-TL1 | MM | A3302G | tRNA | − | + | Cfrm | Pathogenic | 0.0% | 0 (0) | 10 |
| | | | | Leu | | | | | (0.0%) | | |
| | | | | (UUR) | | | | | | | |
| 3303 | MT-TL1 | MMC | C3303T | tRNA | + | + | Cfrm | Pathogenic | 0.0% | 0 (0) | 12 |
| | | | | Leu | | | | | (0.0%) | | |
| | | | | (UUR) | | | | | | | |
| 4298 | MT-TI | CPEO/MS | G4298A | tRNA | − | + | Cfrm | Pathogenic | 0.0% | 0 (0) | 9 |
| | | | | Ile | | | | | (0.0%) | | |
| 4300 | MT-TI | MICM | A4300G | tRNA | + | + | Cfrm | Pathogenic | 0.0% | 0 (0) | 9 |
| | | | | Ile | | | | | (0.0%) | | |
| 4308 | MT-TI | CPEO | G4308A | tRNA | − | + | Cfrm | Pathogenic | 0.0% | 0 (0) | 2 |
| | | | | Ile | | | | | (0.0%) | | |
| 4332 | MT-TO | Encephalopathy/ | G4332A | tRNA | − | + | Cfrm | Pathogenic | 0.0% | 0 (0) | 6 |
| | | MELAS | | Gln | | | | | (0.0%) | | |
| 4450 | MT-TM | Myopathy/MELAS/ | G4450A | tRNA | − | + | Cfrm | Pathogenic | 0.0% | 0 (0) | 4 |
| | | Leigh Syndrome | | Met | | | | | (0.0%) | | |
| 5521 | MT-TW | Mitochondrial | G5521A | tRNA | − | + | Cfrm | Pathogenic | 0.0% | 0 (0) | 5 |
| | | myopathy | | Trp | | | | | (0.0%) | | |
| 5537 | MT-TW | Leigh Syndrome | A5537i | tRNA | − | + | Cfrm | — | 0.0% | 0 (0) | 5 |
| | | | nsT | Trp | | | | | (0.0%) | | |
| 5650 | MT-TA | Myopathy | G5650A | tRNA | − | + | Cfrm | Pathogenic | 0.0% | 1 (0) | 7 |
| | | | | Ala | | | | | (0.0%) | | |
| 5690 | MT-TN | CPEO + ptosis + | A5690G | tRNA | − | + | Cfrm | Pathogenic | 0.0% | 0 (0) | 7 |
| | | proximal myopathy | | Asn | | | | | (0.0%) | | |
| 5703 | MT-TN | CPEO/MM | G5703A | tRNA | − | + | Cfrm | Pathogenic | 0.0% | 0 (0) | 7 |
| | | | | Asn | | | | | (0.0%) | | |
| 5728 | MT-TN | Multiorgan failure/ | T5728C | tRNA | − | + | Cfrm | Pathogenic | 0.0% | 1 (0) | 3 |
| | | myopathy | | Asn | | | | | (0.0%) | | |
| 7445 | MT-TS1 | SNHL | A7445G | tRNA | + | + | Cfrm | — | 0.0% | 1 (0) | 32 |
| | precursor | | | Ser | | | | | (0.0%) | | |
| | | | | (UCN) | | | | | | | |
| | | | | precursor | | | | | | | |
| 7471 | MT-TS1 | PEM/AMDF/ | C7471C | tRNA | + | + | Cfrm | — | 0.0% | 7 (0) | 28 |
| | | Motor neuron | C | Ser | | | | | (0.0%) | | |
| | | disease-like | | (UCN) | | | | | | | |
| 7497 | MT-TS1 | MM/EXIT | G7497A | tRNA | + | + | Cfrm | Pathogenic | 0.0% | 1 (0) | 7 |
| | | | | Ser | | | | | (0.0%) | | |
| | | | | (UCN) | | | | | | | |
| 7510 | MT-TS1 | SNHL | T7510C | tRNA | − | + | Cfrm | Pathogenic | 0.0% | 1 (0) | 13 |
| | | | | Ser | | | | | (0.0%) | | |
| | | | | (UCN) | | | | | | | |
| 7511 | MT-TS1 | SNHL/Deafness | T7511C | tRNA | + | + | Cfrm | Pathogenic | 0.0% | 2 (0) | 20 |
| | | | | Ser | | | | | (0.0%) | | |
| | | | | (UCN) | | | | | | | |
| 8306 | MT-TK | Severe adult-onset | T8306C | tRNA | − | + | Cfrm | Pathogenic | 0.0% | 0 (0) | 3 |
| | | multisymptom | | Lys | | | | | (0.0%) | | |
| | | myopathy/ | | | | | | | | | |
| | | Myoclonic epilepsy | | | | | | | | | |
| 8313 | MT-TK | MNGIE/ | G8313A | tRNA | − | + | Cfrm | Pathogenic | 0.0% | 1 (0) | 6 |
| | | Progressive | | Lys | | | | | (0.0%) | | |
| | | mitocytopathy | | | | | | | | | |
| 8340 | MT-TK | Myopathy/Exercise | G8340A | tRNA | − | + | Cfrm | Pathogenic | 0.0% | 0 (0) | 7 |
| | | Intolerance/Eye | | Lys | | | | | (0.0%) | | |
| | | disease + SNHL | | | | | | | | | |
| 8344 | MT-TK | MERRF; Other - LD/ | A8344G | tRNA | − | + | Cfrm | Pathogenic | 0.0% | 4 (0) | 124 |
| | | Depressive mood | | Lys | | | | | (0.0%) | | |
| | | disorder/ | | | | | | | | | |
| | | leukoencephalopathy/ | | | | | | | | | |
| | | HiCM | | | | | | | | | |
| 8356 | MT-TK | MEPRF | T8356C | tRNA | − | + | Cfrm | Pathogenic | 0.0% | 0 (0) | 10 |
| | | | | Lys | | | | | (0.0%) | | |
| 8363 | MT-TK | MICM + DEAF/ | G8363A | tRNA | − | + | Cfrm | Pathogenic | 0.0% | 0 (0) | 70 |
| | | MERRF/Autism/ | | Lys | | | | | (0.0%) | | |
| | | LS/ | | | | | | | | | |
| | | Ataxia + Lipomas | | | | | | | | | |
| 10010 | MT-TO | PEM | T10010 | tRNA | − | + | Cfrm | Pathogenic | 0.0% | 0 (0) | 9 |
| | | | C | Gly | | | | | (0.0%) | | |
| 12147 | MT-TH | MERRF-MELAS/ | G12147 | tRNA | − | + | Cfrm | Pathogenic | 0.0% | 0 (0) | 5 |
| | | Encephalopathy | A | His | | | | | (0.0%) | | |
| 12258 | MT-TS2 | DMDF/RP +S NHL | C12258 | tRNA | − | + | Cfrm | Pathogenic | 0.0% | 1 (0) | 7 |
| | | | A | Ser | | | | | (0.0%) | | |
| | | | | (AGY) | | | | | | | |
| 12276 | MT-TL2 | CPEO | G12276 | tRNA | − | + | Cfrm | Pathogenic | 0.0% | 1 (0) | 1 |
| | | | A | Leu | | | | | (0.0%) | | |
| | | | | (CUN) | | | | | | | |
| 12294 | MT-TL2 | CPEO/ | G12294 | tRNA | − | + | Cfrm | Pathogenic | 0.0% | 0 (0) | 2 |
| | | EXIT + Ophthalmoplegia | A | Leu | | | | | (0.0%) | | |
| | | | | (CUN) | | | | | | | |
| 12315 | 335.2 | CPEO/KSS/ | G12315 | tRNA | − | + | Cfrm | Pathogenic | 0.0% | 0 (0) | 18 |
| | | possible carotid | A | Leu | | | | | (0.0%) | | |
| | | atherosclerosis risk, | | (CUN) | | | | | | | |
| | | trend toward | | | | | | | | | |
| | | myocardial | | | | | | | | | |
| | | infarction risk | | | | | | | | | |
| 12316 | MT-TL2 | CPEO | G12316 | tRNA | − | + | Cfrm | Pathogenic | 0.0% | 0 (0) | 2 |
| | | | A | Leu | | | | | (0.0%) | | |
| | | | | (CUN) | | | | | | | |
| 14674 | MT-TB | Reversible COX | T14674 | tRNA | + | − | Cfrm | Pathogenic | 0.0% | 7 (0) | 6 |
| | | deficiency myopathy | C | Glu | | | | | (0.0%) | | |
| 14709 | MT-TB | MM + DMDF/ | T14709 | tRNA | + | + | Cfrm | Pathogenic. | 0.0% | 1 (0) | 22 |
| | | Encephalomyopathy/ | C | Glu | | | | | (0.0%) | | |
| | | Dementia + diabetes + | | | | | | | | | |
| | | ophthalmoplegia | | | | | | | | | |
| 14710 | MT-TB | Encephalomyopathy + | G14710 | tRNA | − | + | Cfrm | Pathogenic | 0.0% | 0 (0) | 5 |
| | | Retinopathy | A | Glu | | | | | (0.0%) | | |

Column Heading Key: A: Position; B: Locus; C: Disease; D: Allele; E: RNA; F: Homoplasmy; G: Heteroplasmy; H: Status; I: MitoTip; J: GB Freq FL (CR); K: GB Seqs FL (CR); L: Reference

TABLE 3
MITOMAP: Reported Mitochondrial DNA Base Substitution Diseases: Coding and Control Region Point Mutations

| A | B | C | D | E | F | G | H | I | J | K | L |
|---|---|---|---|---|---|---|---|---|---|---|---|
| 114 | MT- | BD-associated | C114T | C-T | noncoding | + | − | Reported | 0.4% | 216 | 1 |
| | CR | | | | | | | | (0.1%) | (268) | |
| 146 | MT- | Absence of | T146C | T-C | noncoding | + | − | Reported | 19.5% | 9761 | 1 |
| | CR | Endometriosis | | | | | | | (11.6%) | (8181) | |
| 150 | MT- | Longevity/Cervical | C150T | C-T | noncoding | + | + | Conflicting | 13.4% | 6222 | 3 |
| | CR | Carcinoma/HPV | | | | | | reports | (9.6%) | (7062) | |
| | | infection risk | | | | | | | | | |
| 195 | MT- | BD-associated/ | T195C | T-C | noncoding | + | − | Reported | 19.6% | 9817 | 3 |
| | CR | melanoma pts | | | | | | | (11.7%) | (8990) | |
| 302 | MT- | Higher in melanoma | A302ACC | A-ACC | noncoding | · | · | Reported | 0.3% | 067 | 1 |
| | CP | patient group | | | | | | | (0.0%) | (14) | |
| 309 | MT- | AD-weakly | C309CC | C-CC | noncoding | · | · | Reported | 1.1% | 531 | 1 |
| | CP | associated | | | | | | | (1.3%) | (952) | |
| 310 | MT- | Melanoma patients | T310TC | T-TC | noncoding | · | · | Reported | 0.0% | 0 (0) | 1 |
| | CR | | | | | | | | (0.0%) | | |
| 499 | MT- | Endometriosis | G499A | G-A | noncoding | + | − | Reported | 3.7% | 1832 | 1 |
| | CR | | | | | | | | (1.9%) | (1356) | |
| 547 | MT- | Tubulointerstitial | A547T | A-T | noncoding | + | − | Reported | 0.0% | 0 (0) | 1 |
| | | kidney disease | | | | | | | (0.0%) | | |
| 573 | MT- | Absence of | C573CCC | C-CCC | noncoding | + | − | Reported | 0.0% | 0 (20) | 1 |
| | CR | Endometriosis | | | | | | | (0.0%) | | |
| 3308 | MT | MELAS/DEAF | T3308C | T-C | M-T | − | + | P.M.- | 0.7% | 352 | 15 |
| | ND1 | enhancer/ | | | | | | possibly | (0.0%) | (0) | |
| | | hypertension/LVNC/ | | | | | | synergistic | | | |
| | | putative LHON | | | | | | | | | |
| 3308 | MT- | Sudden Infant Death | T3308G | T-G | M-Term | + | + | Reported | 0.0% | 6 (0) | 1 |
| | ND1 | | | | | | | | (0.0%) | | |
| 3310 | MT- | Diabetes/HCM | C3310T | C-T | P-S | + | + | Reported | 0.0% | 12 (0) | 4 |
| | ND1 | | | | | | | | (0.0%) | | |
| 3316 | MT- | Diabetes/LHON/ | G3316A | G-A | A-T | + | − | Unclear | 1.0% | 513 | 21 |
| | ND1 | PEO | | | | | | | (0.0%) | (0) | |
| 3335 | MT- | LHON | T3335C | T-C | I-T | + | − | Reported | 0.1% | 54 (0) | 1 |
| | ND1 | | | | | | | | (0.0%) | | |
| 3336 | MT- | Carotid | T3336C | T-C | I-I | - | + | Reported | (0.0%) | 26 | 2 |
| | ND1 | atherosclerosis risk | | | | | | | (0.0%) | (0) | |
| 3337 | MT- | Cardiomyophathy | G3337A | G-A | V-M | + | − | Possibly | 0.2% | 79 (0) | 2 |
| | ND1 | | | | | | | synergistic | (0.0%) | | |
| 3340 | MT- | Encephalo- | C3340T | C-T | P-S | + | − | Reported | 0.0% | 3 (0) | 2 |
| | ND1 | neuromyopathy | | | | | | | (0.0%) | | |
| 3376 | MT- | LHON MELAS | G3376A | G-A | E-K | + | + | Cfrm | 0.0% | 0 (0) | 3 |
| | ND1 | overlap | | | | | | | (0.0%) | | |
| 3380 | MT- | MELAS | G3380A | G-A | R-Q | − | + | Reported | 0.0% | 3 (0) | 1 |
| | ND1 | | | | | | | | (0.0%) | | |
| 3388 | MT- | Materally Inherited | C3388A | C-A | L-M | · | · | Reported | 0.0% | 17 (0) | 1 |
| | ND1 | Nonsyndromic | | | | | | | (0.0%) | | |
| | | Deafness | | | | | | | | | |
| 3391 | MT- | LHON | G3391A | G-A | G-S | + | − | Reported | 0.1% | 52 (0) | 1 |
| | ND1 | | | | | | | | (0.0%) | | |
| 3394 | MT- | LHON/Diabetes/ | T3394C | T-C | Y-H | + | − | Reported/ | 1.3% | 633 | 32 |
| | ND1 | CPTdeficiency/high | | | | | | Population | (0.0%) | (0) | |
| | | altitude adaptation | | | | | | dependent | | | |
| 3395 | MT- | LHON/HCM with | A3395G | A-G | Y-C | + | + | Reported | 0.0% | 23 (0) | 3 |
| | ND1 | hearing loss | | | | | | | (0.0%) | | |
| 3396 | MT- | NSHL/MIDD | T3396C | T-C | Y-Y | + | − | Reported/ | 0.3% | 462 | 2 |
| | ND1 | | | | | | | Unclear | (0.0%) | (0) | |
| 3397 | MT- | ADPD/Possibly | A3397G | A-G | M-V | + | − | Reported | 0.3% | 150 | 11 |
| | ND1 | LVNC- | | | | | | | (0.0%) | (0) | |
| | | cardiomyopathy | | | | | | | | | |
| | | associated | | | | | | | | | |
| 3398 | MT- | DMDF+HCM/ | T3398C | T-C | M-T | + | − | Reported | 0.4% | 106 | 3 |
| | ND1 | GDM/ | | | | | | | (0.0%) | (0) | |
| | | possibly LVNC | | | | | | | | | |
| | | cardiomyopathy- | | | | | | | | | |
| | | associated | | | | | | | | | |
| 3399 | MT- | Gestational Diabetes | A3399T | A-T | M-I | + | − | Reported | 0.0% | 25 (0) | 1 |
| | ND1 | (GDM) | | | | | | | (0.0%) | | |
| 3407 | MT- | HCM/Muscle | G3407A | G-A | R-H | + | − | Conflicting | 0.0% | 1 (0) | 3 |
| | ND1 | involvement | | | | | | reports | (0.0%) | | |
| 3418 | MT- | AMegL | A3418G | A-G | N-D | + | − | Reported | 0.0% | 1 (0) | 1 |
| | ND1 | | | | | | | | (0.0%) | | |
| 3421 | MT- | MIDD | G3421A | G-A | V-I | + | − | Reported | 0.1% | 20 (0) | 2 |
| | ND1 | | | | | | | | (0.0%) | | |
| 3460 | MT- | LHON | G3460A | G-A | A-T | + | + | Cfrm | 0.0% | 23 (0) | 160 |
| | ND1 | | | | | | | | (0.0%) | | |
| 3472 | MT- | LHON | T3472C | T-C | F-L | + | + | Reported | 0.0% | 5 (0) | 7 |
| | ND1 | | | | | | | | (0.0%) | | |
| 3481 | MT- | MELAS/Progressive | G3481A | G-A | E-K | − | + | Reported | 0.0% | 0 (0) | 3 |
| | ND1 | Encephalomyopathy | | | | | | | (0.0%) | | |
| 3488 | MT- | LHON | T3488C | T-C | L-P | + | − | Reported | 0.0% | 1 (0) | 1 |
| | ND1 | | | | | | | | (0.0%) | | |
| 3496 | MT- | LHON | G3496T | G-T | A-S | + | − | Reported/ | 0.0% | 11 (0) | 3 |
| | ND1 | | | | | | | Secondary | (0.0%) | | |
| 3497 | MT- | LHON | C3497T | C-T | A-V | + | − | Reported/ | 0.4% | 184 | 5 |
| | ND1 | | | | | | | Secondary | (0.0%) | (0) | |
| 3551 | MT- | LHON | C3551T | C-T | A-V | + | − | Reported | 0.0% | 0 (0) | 1 |
| | ND1 | | | | | | | | (0.0%) | | |
| 3571 | MT- | Possible LHON | C3571T | C-T | L-F | · | · | Reported | 0.2% | 122 | 3 |
| | ND1 | helper mut. | | | | | | | (0.0%) | (0) | |
| 3632 | MT- | LHON | C3632T | C-T | S-F | + | − | Reported | 0.0% | 0 (0) | 1 |
| | ND1 | | | | | | | | (0.0%) | | |
| 3634 | MT- | LHON | A3634G | A-G | S-G | + | − | Reported | 0.0% | 0 (0) | 1 |
| | ND1 | | | | | | | | (0.0%) | | |
| 3635 | MT- | LHON | G3635A | G-A | S-N | + | − | Cfrm | 0.0% | 9 (0) | 11 |
| | ND1 | | | | | | | | (0.0%) | | |
| 3644 | MT- | BD-associated | T3644C | T-C | V-A | · | · | Reported | 9.4% | 207 | 4 |
| | ND1 | | | | | | | | [symbol] | (0) | |
| | | | | | | | | | (0.0%) | | |
| 3667 | MT- | Peripheral | T3667G | T-G | W-G | + | − | Reported | 0.0% | 1 (0) | 1 |
| | ND1 | neuropathy | | | | | | | (0.0%) | | |
| | | of T2 diabetes | | | | | | | | | |
| 3688 | MT- | Leigh Syndrome | G3688A | G-A | A-T | + | − | Reported | 0.0% | 0 (0) | 2 |
| | ND1 | | | | | | | | (0.0%) | | |
| 3697 | MT- | MELAS/LS/LDYT/ | G3697A | G-A | G-S | + | + | Cfrm | 0.0% | 0 (0) | 13 |
| | ND1 | BSN | | | | | | | (0.0%) | | |
| 3700 | MT- | LHON | G3700A | G-A | A-T | + | − | Cfrm | 0.0% | 3 (0) | 5 |
| | ND1 | | | | | | | | (0.0%) | | |
| 3713 | MT- | LHON | T3713C | T-C | V-A | + | − | Reported | 0.0% | 0 (0) | 1 |
| | ND1 | | | | | | | | (0.0%) | | |
| 3733 | MT- | LHON | G3733A | G-A | E-K | + | + | Cfrm | 0.0% | 9 (0) | 8 |
| | ND1 | | | | | | | | (0.0%) | | |
| 3733 | MT- | LHON | G3733C | G-C | E-Q | − | + | Reported | 0.0% | 0 (0) | 1 |
| | ND1 | | | | | | | | (0.0%) | | |
| 3736 | MT- | LHON | G3736A | G-A | V-I | · | · | Reported | 0.2% | 82 (0) | 2 |
| | ND1 | | | | | | | | (0.0%) | | |
| 3745 | MT- | LHON/high altitude | G3745A | G-A | A-T | · | · | Reported/ | 0.2% | 102 | 3 |
| | ND1 | variant | | | | | | Population- | (0.0%) | (0) | |
| | | | | | | | | dependent | | | |
| 3769 | MT- | LHON | C3769G | C-G | L-V | + | − | Reported | 0.0% | 0 (0) | 1 |
| | ND1 | | | | | | | | (0.0%) | | |
| 3781 | MT- | LHON | T3781C | T-C | S-P | + | − | Reported | 0.0% | 0 (0) | 1 |
| | ND1 | | | | | | | | (0.0%) | | |
| 3796 | MT- | Adult-Onset Dystonia | A3796G | A-G | T-A | − | + | Reported | 0.5% | 236 | 4 |
| | ND1 | | | | | | | | (0.0%) | (0) | |
| 3833 | MT- | PEG | T3833A | T-A | L-Q | + | − | Reported | 0.0% | 0 (0) | 2 |
| | ND1 | | | | | | | | (0.0%) | | |
| 3866 | MT- | LHON + limb | T3866C | T-C | I-T | · | · | Reported | 0.3% | 143 | 5 |
| | ND1 | claudication | | | | | | | (0.0%) | (0) | |
| 3890 | MT- | Progressive | G3890A | G-A | R-Q | − | + | Cfrm | 0.0% | 1 (0) | 7 |
| | ND1 | Encephalomyopathy/ | | | | | | | (0.0%) | | |
| | | LS/Optic Atrophy | | | | | | | | | |
| 3902 | MT- | EXIT + myalgia/ | 3902_3908inv | ACCTTGC- | DLA- | − | + | Cfrm | 0.0% | 0 (0) | 3 |
| | ND1 | severe LA + cardiac/3- | | GCAAGGT | GKV | | | | (0.0%) | | |
| | | MGA aciduria | | | | | | | | | |
| 3919 | MT- | LHON | T3919C | T-C | S-P | + | − | Reported | 0.0% | 0 (0) | 1 |
| | ND1 | | | | | | | | (0.0%) | | |
| 3945 | MT- | Leigh-like phenotype | C3945A | C-A | I-M | · | · | Reported | 0.0% | 0 (0) | 1 |
| | ND1 | | | | | | | | (0.0%) | | |
| 3946 | MT- | MELAS | G3946A | G-A | E-K | + | + | Reported | 0.0% | 2 (0) | 7 |
| | ND1 | | | | | | | | (0.0%) | | |
| 3949 | MT- | MELAS | T3949C | T-C | Y-H | − | + | Reported | 0.0% | 1 (0) | 7 |
| | ND1 | | | | | | | | (0.0%) | | |
| 3958 | MT- | LHON | G3958A | G-A | G-S | + | − | Reported | 0.0% | 0 (0) | 1 |
| | ND1 | | | | | | | | (0.0%) | | |
| 3959 | MT- | MELAS | G3959A | G-A | G-D | · | · | Reported | 0.0% | 0 (0) | 1 |
| | ND1 | | | | | | | | (0.0%) | | |
| 3995 | MT- | MELAS | A3995G | A-G | N-S | · | · | Reported | 0.0% | 18 (0) | 2 |
| | ND1 | | | | | | | | (0.0%) | | |
| 4081 | MT- | LHON | T4081C | T-C | F-L | + | − | Reported | 0.0% | 0 (0) | 1 |
| | ND1 | | | | | | | | (0.0%) | | |
| 4123 | MT- | LHON | A4123T | A-T | I-F | + | − | Reported | 0.0% | 0 (0) | 1 |
| | ND1 | | | | | | | | (0.0%) | | |
| 4132 | MT- | NAION-associated | G4132A | G-A | A-T | + | − | Reported | 0.0% | 7 (0) | 2 |
| | ND1 | | | | | | | | (0.0%) | | |
| 4136 | MT- | 112229 | A4136G | A-G | Y-C | + | − | Possibly | 0.1% | 66 (0) | 11 |
| | ND1 | | | | | | | synergistic | (0.0%) | | |
| 4142 | MT- | Developmental delay, | G4142A | G-A | R-Q | − | + | Reported | 0.0% | 0 (0) | 2 |
| | ND1 | seizure, hypotonia | | | | | | | (0.0%) | | |
| 4160 | MT- | LHON/LHON plus | T4160C | T-C | L-P | + | − | Reported | 0.0% | 1 (0) | 12 |
| | ND1 | | | | | | | | (0.0%) | | |
| 4163 | MT- | LHON | T4163C | T-C | M-T | + | − | Reported | 0.0% | 0 (0) | 1 |
| | ND1 | | | | | | | | (0.0%) | | |
| 4171 | MT- | LHON/Leigh-like | C4171A | C-A | L-M | + | + | Cfrm | 0.0% | 2 (0) | 10 |
| | ND1 | phenotype | | | | | | | (0.0%) | | |
| 4216 | MT- | LHON/Insulin | T4216C | T-C | Y-H | + | − | Reported | 9.9% | 4952 | 42 |
| | ND1 | Resistance/possible | | | | | | | (0.0%) | (0) | |
| | | adaptive high altitude | | | | | | | | | |
| | | variant | | | | | | | | | |
| 4633 | MT- | LHON candidate | C4633G | C-G | A-G | + | − | Reported | 0.0% | 0 (0) | 1 |
| | ND2 | | | | | | | | (0.0%) | | |
| 4640 | MT- | LHON/Epilepsy | C4640A | C-A | I-M | + | − | Reported | 0.4% | 177 | 8 |
| | ND2 | | | | | | | | (0.0%) | (0) | |
| 4648 | MT- | PEG | T4648C | T-C | F-S | + | − | Reported | 0.0% | 1 (0) | 2 |
| | ND2 | | | | | | | | (0.0%) | | |
| 4659 | MT- | possible PD risk factor | G4659A | G-A | A-T | + | − | Reported | 0.1% | 65 (0) | 1 |
| | ND2 | | | | | | | | (0.0%) | | |
| 4681 | MT- | Leigh Syndrome | T4681C | T-C | L-P | − | + | Reported | 0.0% | 1 (0) | 3 |
| | ND2 | | | | | | | | (0.0%) | | |
| 4769 | MT- | SZ-associated | A4769A | A-A | M-M | + | − | Reported | 2.3% | 1167 | 2 |
| | ND2 | | | | | | | | (0.0%) | (0) | |
| 4833 | MT- | Diabetes helper | A4833G | A-G | T-A | + | − | Reported | 0.9% | 452 | 3 |
| | ND2 | mutation AD, PD | | | | | | | (0.0%) | (0) | |
| 4852 | MT- | LHON | T4852A | T-A | L-Q | + | − | Reported | 0.0% | 0 (0) | 1 |
| | ND2 | | | | | | | | (0.0%) | | |
| 4883 | MT- | Glaucoma | C4883T | C-T | P-P | + | − | Conflicting | 4.8% | 2393 | 2 |
| | ND2 | | | | | | | reports | (0.0%) | (0) | |
| 4917 | MT- | LHON/Insulin | A4917G | A-G | N-D | + | − | Reported | 4.8% | 2390 | 28 |
| | ND2 | Resistance/AMD/ | | | | | | | (0.0%) | (0) | |
| | | NRTI-PN | | | | | | | | | |
| 5001 | MT- | Developmental delay, | A5001AA | A-AA | frameshift | − | + | Reported | 0.0% | 0 (0) | 2 |
| | ND2 | seizure, | | | | | | | (0.0%) | | |
| | | cardiomyopathy, lactic | | | | | | | | | |
| | | acidosis | | | | | | | | | |
| 5095 | MT- | Proximal muscle | T5095C | T-C | I-T | · | · | Reported | 0.0% | 20 (0) | 1 |
| | ND2 | weakness and atrophy | | | | | | | (0.0%) | | |
| 5133 | MT- | Exercise intolerance | 5133_5134delAA | AA-del | frameshift | · | · | Reported | 0.0% | 0 (0) | 5 |
| | ND2 | (EXIT) | | | | | | | (0.0%) | | |
| 5178 | MT- | Longevity/ | C5178A | C-A | L-M | + | − | Reported | 4.7% | 2370 | 23 |
| | ND2 | Extraversion/diabetes/ | | | | | | | (0.0%) | (0) | |
| | | AMS protection/ | | | | | | | | | |
| | | blood iron metabolism/ | | | | | | | | | |
| | | correlation with | | | | | | | | | |
| | | myocardial infarction/ | | | | | | | | | |
| | | atherosclerosis | | | | | | | | | |
| 5244 | MT- | LHON | G5244A | G-A | G-S | − | + | Reported | 0.0% | 0 (0) | 2 |
| | ND2 | | | | | | | | (0.0%) | | |
| 5452 | MT- | Progressive | C5452T | C-T | T-M | + | − | Reported | 0.0% | 15 (0) | 2 |
| | ND2 | Encephalomyopathy | | | | | | | (0.0%) | | |
| 5460 | MT- | AD/PD | G5460A | G-A | A-T | + | + | Conflicting | 6.5% | 3272 | 9 |
| | ND2 | | | | | | | reports | (0.0%) | (0) | |
| 5460 | MT- | AD | G5460T | G-T | A-S | + | + | Reported | 0.0% | 0 (0) | 5 |
| | ND2 | | | | | | | | (0.0%) | | |
| 5911 | MT- | Prostate Cancer | C5911T | C-T | A-V | + | − | Reported | 0.5% | 248 | 1 |
| | CO1 | | | | | | | | (0.0%) | (0) | |
| 5913 | MT- | Prostate Cancer/ | G5913A | G-A | D-N | + | − | Reported | 1.0% | 482 | 3 |
| | CO1 | hypertension | | | | | | | (0.0%) | (0) | |
| 5920 | MT- | Myoglobinuria/EXIT | G5920A | G-A | W-Ter | − | + | Reported | 0.0% | 0 (0) | 4 |
| | CO1 | | | | | | | | (0.0%) | | |
| 5935 | MT- | Prostate Cancer | A5935G | A-G | N-S | + | − | Reported | 0.0% | 1 (0) | 1 |
| | CO1 | | | | | | | | (0.0%) | | |
| 5973 | MT- | Prostate Cancer | G5973A | G-A | A-T | + | − | Reported | 0.0% | 11 (0) | 1 |
| | CO1 | | | | | | | | (0.0%) | | |
| 6020 | MT- | Motor Neuron Disease | 6020_6024delCG | CGAGC-del | AELGQ- | − | + | Reported | 0.0% | 0 (0) | 1 |
| | CO1 | | AGC | | AGPATer | | | | (0.0%) | | |
| 6081 | MT- | Prostate Cancer | G6081A | G-A | A-T | + | − | Reported | 0.0% | 1 (0) | 1 |
| | CO1 | | | | | | | | (0.0%) | | |
| 6150 | MT- | Prostate Cancer/ | G6150A | G-A | V-I | + | − | Reported | 0.5% | 233 | 2 |
| | CO1 | enriched in POAG | | | | | | | (0.0%) | (0) | |
| | | cohort | | | | | | | | | |
| 6253 | MT- | Prostate Cancer/ | T6253C | T-C | M-T | + | − | Reported | 1.0% | 524 | 3 |
| | CO1 | enriched in POAG | | | | | | | (0.0%) | (0) | |
| | | cohort | | | | | | | | | |
| 6261 | MT- | Prostate Cancer/ | G6261A | G-A | A-T | + | − | Reported | 0.7% | 361 | 3 |
| | CO1 | LHON | | | | | | | (0.0%) | (0) | |
| 6267 | MT- | Prostate Cancer | G6267A | G-A | A-T | + | − | Reported | 0.2% | 77 (0) | 1 |
| | CO1 | | | | | | | | (0.0%) | | |
| 6285 | MT- | Prostate Cancer | G6285A | G-A | V-I | + | − | Reported | 0.2% | 121 | 1 |
| | CO1 | | | | | | | | (0.0%) | (0) | |
| 6307 | MT- | Asthenozoospermic | A6307G | A-G | N-S | · | + | Reported | 0.0% | 2 (0) | 1 |
| | CO1 | infertility | | | | | | | (0.0%) | | |
| 6328 | MT- | EXIT (Exercise | C6328T | C-T | S-F | + | − | Reported | 0.0% | 0 (0) | 2 |
| | CO1 | Intolerance) | | | | | | | (0.0%) | | |
| 6340 | MT- | Prostate Cancer | C6340T | C-T | T-I | + | − | Reported | 0.2% | 82 (0) | 2 |
| | CO1 | | | | | | | | (0.0%) | | |
| 6459 | MT- | Sepsis susceptibility | T6459C | T-C | W-R | + | − | Reported | 0.0% | 0 (0) | 1 |
| | CO1 | | | | | | | | (0.0%) | | |
| 6480 | MT- | Prostate Cancer/ | G6480A | G-A | V-I | + | − | Reported | 0.3% | 146 | 4 |
| | CO1 | enriched in POAG | | | | | | | (0.0%) | (0) | |
| | | cohort | | | | | | | | | |
| 6489 | MT- | CO1 deficiency with | C6489A | C-A | L-I | − | + | Reported | 0.2% | 86 (0) | 3 |
| | CO1 | epilepsia partialis | | | | | | | (0.0%) | | |
| | | continua | | | | | | | | | |
| 6597 | MT- | MELAS-like | C6597A | C-A | Q-K | − | + | Reported | 0.0% | 0 (0) | 1 |
| | CO1 | syndrome | | | | | | | (0.0%) | | |
| 6663 | MT- | Prostate Cancer | A6663G | A-G | I-V | + | − | Reported | 0.3% | 151 | 3 |
| | CO1 | | | | | | | | (0.0%) | (0) | |
| 6698 | MT- | Myopathy | A6698del | A-del | K- | − | + | Reported | 0.0% | 0 (0) | 1 |
| | CO1 | | | | K_frame- | | | | (0.0%) | | |
| | | | | | shift | | | | | | |
| 6708 | MT- | MM & | G6708A | G-A | G-Ter | − | + | Reported | 0.0% | 0 (0) | 1 |
| | CO1 | Rhabdomyolysis | | | | | | | (0.0%) | | |
| 6721 | MT- | Acquired Idiopathic | T6721C | T-C | M-T | − | + | Reported | 0.0% | 0 (0) | 2 |
| | CO1 | Sideroblastic Anemia | | | | | | | (0.0%) | | |
| 6742 | MT- | Acquired Idiopathic | T6742C | T-C | I-T | − | + | Reported | 0.0% | 0 (0) | 2 |
| | CO1 | Sideroblastic Anemia | | | | | | | (0.0%) | | |
| 6860 | MT- | Dilated | A6860C | A-C | K-N | + | − | Reported | 0.0% | 0 (0) | 1 |
| | CO1 | Cardiomyopathy | | | | | | | (0.0%) | | |
| 6930 | MT- | Multisystem Disorder | G6930A | G-A | G-Ter | − | + | Reported | 0.0% | 0 (0) | 3 |
| | CO1 | | | | | | | | (0.0%) | | |
| 6955 | MT- | Mild EXIT and MR | G6955A | G-A | G-D | + | + | Reported | 0.0% | 1 (0) | 1 |
| | CO1 | | | | | | | | (0.0%) | | |
| 6962 | MT- | Possible helper variant | G6962A | G-A | L-L | + | − | Reported | 2.4% | 1206 | 1 |
| | CO1 | for 15927A | | | | | | | (0.0%) | (0) | |
| 7023 | MT- | MELAS-like | G7023A | G-A | V-M | − | + | Reported | 0.0% | 1 (0) | 1 |
| | CO1 | syndrome | | | | | | | (0.0%) | | |
| 7041 | MT- | Prostate Cancer | G7041A | G-A | V-I | + | − | Reported | 0.0% | 6 (0) | 1 |
| | CO1 | | | | | | | | (0.0%) | | |
| 7080 | MT- | Prostate Cancer | T7080C | T-C | F-L | + | − | Reported | 0.1% | 55 (0) | 1 |
| | CO1 | | | | | | | | (0.0%) | | |
| 7083 | MT- | Prostate Cancer | A7083G | A-G | I-V | + | − | Reported | 0.0% | 15 (0) | 1 |
| | CO1 | | | | | | | | (0.0%) | | |
| 7158 | MT- | Prostate Cancer | A7158G | A-G | I-V | + | − | Reported | 0.1% | 36 (0) | 1 |
| | CO1 | | | | | | | | (0.0%) | | |
| 7305 | MT- | Prostate Cancer | A7305C | A-C | M-L | + | − | Reported | 0.0% | 0 (0) | 1 |
| | CO1 | | | | | | | | (0.0%) | | |
| 7402 | MT- | Isolated complex IV | C7402del | C-del | frameshift | − | + | Reported | 0.0% | 0 (0) | 1 |
| | CO1 | deficiency | | | | | | | (0.0%) | | |
| 7443 | MT- | DEAF | A7443G | A-G | Ter-G | + | − | Reported | 0.0% | 1 (0) | 4 |
| | CO1 | | | | | | | | (0.0%) | | |
| 7444 | MT- | LHON/SNHL/ | G7444A | G-A | Ter-K | + | − | Reported | 0.4% | 183 | 26 |
| | CO1 | DEAF | | | | | | | (0.0%) | (0) | |
| 7445 | MT- | DEAF | A7445C | A-C | Ter-S | + | − | Reported | 0.0% | 13 (0) | 2 |
| | CO1 | | | | | | | | (0.0%) | | |
| 7445 | MT- | SNHL | A7445G | A-G | Ter-Ter | + | + | Cfrm | 0.0% | 1 (0) | 32 |
| | CO1 | | | | | | | | (0.0%) | | |
| 7587 | MT- | Mitochondrial | T7587C | T-C | M-T | − | + | Reported | 0.0% | 0 (0) | 2 |
| | CO2 | Encephalomyopathy | | | | | | | (0.0%) | | |
| 7598 | MT- | Possible LHON | G7598A | G-A | A-T | − | + | Reported | 1.2% | 608 | 2 |
| | CO2 | helper variant | | | | | | | (0.0%) | (0) | |
| 7623 | MT- | LHON | C7623T | C-T | T-I | + | − | Reported | 0.0% | 0 (0) | 1 |
| | CO2 | | | | | | | | (0.0%) | | |
| 7630 | MT- | MELAS | T7630del | T-del | frameshift | − | + | Reported | 0.0% | 0 (0) | 1 |
| | CO2 | | | | | | | | (0.0%) | | |
| 7637 | MT- | PD risk factor | G7637A | G-A | E-K | − | + | Reported | 0.0% | 2 (0) | 1 |
| | CO2 | | | | | | | | (0.0%) | | |
| 7671 | MT- | MM | T7671A | T-A | M-K | − | + | Reported | 0.0% | 0 (0) | 2 |
| | CO2 | | | | | | | | (0.0%) | | |
| 7697 | MT- | Possible HCM | G7697A | G-A | V-I | + | − | Reported | 0.5% | 253 | 3 |
| | CO2 | susceptibility | | | | | | | (0.0%) | (0) | |
| 7706 | MT- | Alpers-Huttenlocher- | G7706A | G-A | A-T | | + | Reported | 0.0% | 9 (0) | 1 |
| | CO2 | like | | | | | | | (0.0%) | | |
| 7859 | MT- | Progressive | G7859A | G-A | D-N | + | − | Reported | 0.3% | 150 | 1 |
| | CO2 | Encephalomyopathy | | | | | | | (0.0%) | (0) | |
| 7868 | MT- | LHON | C7868T | C-T | L-F | + | − | Possibly | 0.0% | 17 (0) | 1 |
| | CO2 | | | | | | | synergistic | (0.0%) | | |
| 7877 | MT- | PEG glaucoma | A7877C | A-C | K-Q | + | − | Reported | 0.0% | 0 (0) | 1 |
| | CO2 | | | | | | | | (0.0%) | | |
| 7896 | MT- | Multisystem Disorder | G7896A | G-A | W-Ter | − | + | Reported | 0.0% | 0 (0) | 1 |
| | CO2 | | | | | | | | (0.0%) | | |
| 7965 | MT- | Hepatic failure/COX | T7965C | T-C | F-S | · | + | Reported | 0.0% | 1 (0) | 3 |
| | CO2 | deficiency | | | | | | | (0.0%) | | |
| 7970 | MT- | Encephalopathy | G7970T | G-T | E-Ter | − | + | Reported | 0.0% | 0 (0) | 1 |
| | CO2 | | | | | | | | (0.0%) | | |
| 7989 | MT- | Rhabdomyolysis | T7989C | T-C | L-P | − | + | Reported | 0.0% | 0 (0) | 2 |
| | CO2 | | | | | | | | (0.0%) | | |
| 8010 | MT- | Developmental delay, | T8010C | T-C | V-A | − | + | Reported | 0.0% | 2 (0) | 1 |
| | CO2 | ataxia, seizure, | | | | | | | (0.0%) | | |
| | | hypotonia, lactic | | | | | | | | | |
| | | acidosis | | | | | | | | | |
| 8021 | MT- | Asthenozoospermia | A8021G | A-G | I-V | + | − | Reported | 0.0% | 4 (0) | 1 |
| | CO2 | | | | | | | | (0.0%) | | |
| 8042 | MT- | Lactic Acidosis | 8042_8403delAT | AT-del | frameshift | − | + | Reported | 0.0% | 0 (0) | 1 |
| | CO2 | | | | | | | | (0.0%) | | |
| 8078 | MT- | DEAF | G8078A | G-A | V-I | + | − | Reported | 0.1% | 27 (0) | 2 |
| | CO2 | | | | | | | | (0.0%) | | |
| 8088 | MT- | Mitochondrial | T8088del | T-del | frameshift | − | + | Reported | 0.0% | 0 (0) | 1 |
| | CO2 | myopathy with | | | | | | | (0.0%) | | |
| | | complex IV | | | | | | | | | |
| | | deficiency | | | | | | | | | |
| 8108 | MT- | SNHL | A8108G | A-G | I-V | + | − | Reported | 0.1% | 71 (0) | 1 |
| | CO2 | | | | | | | | (0.0%) | | |
| 8119 | MT- | Biliary atresia | T8119del | T-del | frameshift | − | + | Reported | 0.0% | 0 (0) | 1 |
| | CO2 | | | | | | | | (0.0%) | | |
| 8156 | MT- | Multi-system | G8156del | G-del | frameshift | − | + | Reported | 0.0% | 0 (0) | 1 |
| | CO2 | mitochondrial | | | | | | | (0.0%) | | |
| | | disorder | | | | | | | | | |
| 8241 | MT- | MIDD + | T8241G | T-G | F-C | − | + | Conflicting | 0.0% | 0 (0) | 2 |
| | CO2 | retinopathy | | | | | | reports | (0.0%) | | |
| 8249 | MT- | Mitochondrial | G8249A | G-A | G-Ter | + | − | Reported | 0.0% | 1 (0) | 2 |
| | CO2 | myopathy | | | | | | | (0.0%) | | |
| 8381 | MT- | MIDD/LVNC | A8381G | A-G | T-A | + | − | Reported | 0.0% | 13 (0) | 2 |
| | ATP8 | cardiomyopathy-assoc. | | | | | | | (0.0%) | | |
| 8393 | MT- | Reversible brain | C8393T | C-T | P-S | − | + | Reported | 0.3% | 174 | 2 |
| | ATP8 | pseudoatrophy | | | | | | | (0.0%) | (0) | |
| 8403 | MT- | Episodic weakness and | T8403C | T-C | I-T | + | − | Reported | 0.0% | 3 (0) | 1 |
| | ATP8 | progressive | | | | | | | (0.0%) | | |
| | | neuropathy | | | | | | | | | |
| 8411 | MT- | Severe mitochondrial | A8411G | A-G | M-V | + | − | Reported | 0.0% | 2 (0) | 1 |
| | ATP8 | disorder | | | | | | | (0.0%) | | |
| 8414 | MT- | Increased risk of | C8414T | C-T | L-F | + | − | Reported | 3.9% | 1961 | 1 |
| | ATP8 | T2DM in haplogroup | | | | | | | (0.0%) | (0) | |
| | | D4/Longevity | | | | | | | | | |
| 8481 | MT- | Tetralogy of Fallot | C8481T | C-T | P-L | + | − | Reported | 0.0% | 8 (0) | 1 |
| | ATP8 | patient | | | | | | | (0.0%) | | |
| 8490 | MT- | Peripheral neuropathy | T8490C | T-C | M-T | + | − | Reported | 0.1% | 27 (0) | 4 |
| | ATP8 | of T2DM | | | | | | | (0.0%) | | |
| 8519 | MT- | Susceptibility to | G8519A | G-A | E-K | + | − | Reported | 0.2% | 117 | 1 |
| | ATP8 | bullous pemphigoid | | | | | | | (0.0%) | (0) | |
| 8527 | MT- | Neuromuscular | A8527G | A-G | ATP8:K-K | + | − | Reported | 0.4% | 212 | 1 |
| | ATP8/ | disorder, possible | | | ATP6:M-V | | | | (0.0%) | (0) | |
| | 6 | helper mutation | | | | | | | | | |
| 8528 | MT- | Infantile | T8528C | T-C | ATP8:W-R | + | + | Cfrm | 0.0% | 0 (0) | 3 |
| | ATP8/ | cardiomyopathy | | | ATP6:M-T | | | | (0.0%) | | |
| | 6 | | | | | | | | | | |
| 8529 | MT- | Apical HCM | G8529A | G-A | ATP8:W-R | + | − | Reported | 0.0% | 0 (0) | 1 |
| | ATP8/ | | | | ATP6:M-M | | | | (0.0%) | | |
| | 6 | | | | | | | | | | |
| 8558 | MT- | Possibly LVNC | C8558T | C-T | ATP8:P-S | + | − | Reported | 0.0% | 12 (0) | 1 |
| | ATP8/ | cardiomyopathy- | | | ATP6:A-V | | | | (0.0%) | | |
| | 6 | associated | | | | | | | | | |
| 8561 | MT- | Ataxia w neuropathy, | C8561G | C-G | ATP8:P-A | + | + | Reported | 0.0% | 0 (0) | 1 |
| | ATP8/ | DM, SNHL, and | | | ATP6:P-R | | | | (0.0%) | | |
| | 6 | hypogonadism | | | | | | | | | |
| 8561 | MT- | Ataxia w psychomotor | C8561T | C-T | ATP8:P-S | − | + | Reported | 0.0% | 0 (0) | 1 |
| | ATP8/ | delay | | | ATP6:P-L | | | | (0.0%) | | |
| | 6 | | | | | | | | | | |
| 8611 | MT- | Ataxia, microcephaly, | C8611CC | C-CC | frameshift | − | + | Reported | 0.0% | 0 (0) | 2 |
| | ATP6 | developmental delay, | | | | | | | (0.0%) | | |
| | | intellectual disability | | | | | | | | | |
| 8618 | MT- | NARP | T8618TT | T-TT | frameshift | − | + | Reported | 0.0% | 0 (0) | 1 |
| | ATP6 | | | | | | | | (0.0%) | | |
| 8668 | MT- | LHON | T8668C | T-C | W-R | + | − | Reported | 0.1% | 34 (0) | 1 |
| | ATP6 | | | | | | | | (0.0%) | | |
| 8719 | MT- | Suspected mito | G8719A | G-A | G-Ter | − | + | Reported | 0.0% | 0 (0) | 1 |
| | ATP6 | disease | | | | | | | (0.0%) | | |
| 8741 | MT- | MILS protective | T8741G | T-G | L-R | − | + | Reported | 0.0% | 0 (0) | 1 |
| | ATP6 | factor | | | | | | | (0.0%) | | |
| 8794 | MT- | Exercise Endurance/ | C8794T | C-T | H-Y | + | − | Reported | 2.8% | 1399 | 2 |
| | ATP6 | Coronary | | | | | | | (0.0%) | (0) | |
| | | Atherosclerosis risk | | | | | | | | | |
| 8795 | MT- | MILS protective | A8795G | A-G | H-R | − | + | Reported | 0.0% | 0 (0) | 1 |
| | ATP6 | factor | | | | | | | (0.0%) | | |
| 8821 | MT- | Possible LHON | T8821G | T-G | S-A | · | · | Reported | 0.0% | 0 (0) | 1 |
| | ATP6 | helper variant | | | | | | | (0.0%) | | |
| 8836 | MT- | LHON | A8836G | A-G | M-V | + | − | Reported | 0.3% | 132 | 2 |
| | ATP6 | | | | | | | | (0.0%) | (0) | |
| 8851 | MT- | BSN/Leigh syndrome | T8851C | T-C | W-R | + | + | Cfrm | 0.0% | 3 (0) | 6 |
| | ATP6 | | | | | | | | (0.0%) | | |
| 8890 | MT- | Juvenile-onset | A8890G | A-G | K-E | − | + | Reported | 0.0% | 0 (0) | 1 |
| | ATP6 | metabolic syndrome | | | | | | | (0.0%) | | |
| 8932 | MT- | Prostate Cancer/ | C8932T | C-T | P-S | + | − | Reported | 0.4% | 212 | 3 |
| | ATP6 | Neuromuscular | | | | | | | (0.0%) | (0) | |
| | | disorder | | | | | | | | | |
| 8950 | MT- | LDYT | G8950A | G-A | V-I | + | − | Reported | 0.1% | 74 (0) | 2 |
| | ATP6 | | | | | | | | (0.0%) | | |
| 8959 | MT- | Developmental delay, | G8959A | G-A | E-K | + | + | Reported | 0.0% | 4 (0) | 2 |
| | ATP6 | intellectual disability, | | | | | | | (0.0%) | | |
| | | low citrilline | | | | | | | | | |
| 8969 | MT- | Mitochondrial | G8969A | G-A | S-N | − | + | Cfrm | 0.0% | 0 (0) | 4 |
| | ATP6 | myopathy, lactic | | | | | | | (0.0%) | | |
| | | acidosis and | | | | | | | | | |
| | | sideroblastic anemia | | | | | | | | | |
| | | (MLASA)/IgG | | | | | | | | | |
| | | nephropathy | | | | | | | | | |
| 8993 | MT- | NARP/Leigh Disease/ | T8993C | T-C | L-P | − | + | Cfrm | 0.0% | 2 (0) | 36 |
| | ATP6 | MILS/other | | | | | | | (0.0%) | | |
| 8993 | MT- | NARP/Leigh Disease/ | T8993G | T-G | L-R | + | + | Cfrm | 0.0% | 6 (0) | 114 |
| | ATP6 | MILS/other | | | | | | | (0.0%) | | |
| 9010 | MT- | Unspecified | G9010A | G-A | A-T | − | + | Reported | 0.1% | 27 (0) | 1 |
| | ATP6 | neurological disorder | | | | | | | (0.0%) | | |
| 9016 | MT- | LHON | A9016G | A-G | I-V | − | + | Reported | 0.0% | 13 (0) | 2 |
| | ATP6 | | | | | | | | (0.0%) | | |
| 9017 | MT- | Unspecified | T9017C | T-C | I-T | − | + | Reported | 0.0% | 11 (0) | 1 |
| | ATP6 | neurological disorder | | | | | | | (0.0%) | | |
| 9025 | MT- | Motor neuropathy, | G9025A | G-A | G-S | + | − | Reported | 0.1% | 29 (0) | 1 |
| | ATP6 | LS- | | | | | | | (0.0%) | | |
| | | like, colon cancer | | | | | | | | | |
| 9029 | MT- | LHON-like | A9029G | A-G | H-R | + | + | Reported | 0.0% | 1 (0) | 1 |
| | ATP6 | | | | | | | | (0.0%) | | |
| 9032 | MT- | NARP | T9032C | T-C | L-P | − | + | Reported | 0.0% | 0 (0) | 1 |
| | ATP6 | | | | | | | | (0.0%) | | |
| 9035 | MT- | Ataxia syndromes | T9035C | T-C | L-P | + | + | Cfrm | 0.0% | 0 (0) | 2 |
| | ATP6 | | | | | | | | (0.0%) | | |
| 9055 | MT- | PD protective factor | G9055A | G-A | A-T | + | − | Reported | 4.2% | 2067 | 2 |
| | ATP6 | | | | | | | | (0.0%) | (0) | |
| 9058 | MT- | Possibly LVNC | A9058G | A-G | T-A | + | − | Reported | 0.1% | 28 (0) | 1 |
| | ATP6 | cardiomyopathy- | | | | | | | (0.0%) | | |
| | | associated | | | | | | | | | |
| 9071 | MT- | Potentially functional | C9071T | C-T | S-L | + | − | Reported | 0.0% | 14 (0) | 1 |
| | ATP6 | variant cosegregating | | | | | | | (0.0%) | | |
| | | with LHON3635A | | | | | | | | | |
| 9098 | MT- | Predisposition to anti- | T9098C | T-C | I-T | + | − | Reported | 0.1% | 52 (0) | 1 |
| | ATP6 | retroviral mito disease | | | | | | | (0.0%) | | |
| 9101 | MT- | LHON | T9101C | T-C | I-T | + | − | Reported | 0.1% | 37 (0) | 2 |
| | ATP6 | | | | | | | | (0.0%) | | |
| 9127 | MT- | NARP | 9127_9128delAT | AT-del | IL-PTer | − | + | Reported | 0.0% | 0 (0) | 1 |
| | ATP6 | | | | | | | | (0.0%) | | |
| 9134 | MT- | Hypotonia, lactic | A9134G | A-G | E-G | · | · | Reported | 0.0% | 0 (0) | 1 |
| | ATP6 | acidosis, HCM, IUGR | | | | | | | (0.0%) | | |
| 9139 | MT- | 112229 | G9139A | G-A | A-T | + | − | Reported | 0.1% | 40 (0) | 1 |
| | ATP6 | | | | | | | possibly | (0.0%) | | |
| | | | | | | | | synergistic | | | |
| 9155 | MT- | MIDD, renal | A9155G | A-G | Q-R | − | + | Cfrm | 0.0% | 0 (0) | 3 |
| | ATP6 | insufficiency | | | | | | | (0.0%) | | |
| 9155 | MT- | Developmental delay, | A9155T | A-T | Q-L | + | + | Reported | 0.0% | 0 (0) | 1 |
| | ATP6 | intellectual disability, | | | | | | | (0.0%) | | |
| | | low citrilline | | | | | | | | | |
| 9176 | MT- | FBSN/Leigh Disease | T9176C | T-C | L-P | + | + | Cfrm | 0.0% | 3 (0) | 21 |
| | ATP6 | | | | | | | | (0.0%) | | |
| 9176 | MT- | Leigh Disease/ | T9176G | T-G | L-R | + | + | Cfrm | 0.0% | 1 (0) | 9 |
| | ATP6 | Spastic Paraplegia | | | | | | | (0.0%) | | |
| 9185 | MT- | Leigh Disease/Ataxia | T9185C | T-C | L-P | + | + | Cfrm | 0.0% | 3 (0) | 16 |
| | ATP6 | syndromes/NARP- | | | | | | | (0.0%) | | |
| | | like disease | | | | | | | | | |
| 9191 | MT- | Leigh Disease | T9191C | T-C | L-P | − | + | Reported | 0.0% | 0 (0) | 1 |
| | ATP6 | | | | | | | | (0.0%) | | |
| 9205 | MT- | Encephalopathy/ | 9205_9206delTA | TA-del | Ter-M | + | − | Cfrm | 0.0% | 0 (0) | 7 |
| | ATP6 | Seizures/ | | | | | | | (0.0%) | | |
| | | Lacticacidemia | | | | | | | | | |
| 9267 | MT- | MIDD | G9267C | G-C | A-P | − | + | Reported | 0.0% | 0 (0) | 1 |
| | CO3 | | | | | | | | (0.0%) | | |
| 9379 | MT- | MM w lactic acidosis | G9379A | G-A | W-Ter | − | + | Reported | 0.0% | 0 (0) | 1 |
| | CO3 | | | | | | | | (0.0%) | | |
| 9387 | MT- | Asthenozoospermia | G9387A | G-A | V-M | − | + | Reported | 0.0% | 0 (0) | 1 |
| | CO3 | | | | | | | | (0.0%) | | |
| 9438 | MT- | LHON/gout | G9438A | G-A | G-S | + | − | Conflicting | 1.1% | 559 | 14 |
| | CO3 | | | | | | | reports | (0.0%) | (0) | |
| 9478 | MT- | Leigh Disease | T9478C | T-C | V-A | − | + | Reported | 0.0% | 18 (0) | 2 |
| | CO3 | | | | | | | | (0.0%) | | |
| 9480 | MT- | Myoglobinuria | 9480_9494del15 | TTTTTCTTCGCA | FFFAG- | − | + | Reported | 0.0% | 0 (0) | 5 |
| | CO3 | | | GGA-del (SEQ ID | del | | | | (0.0%) | | |
| | | | | NO: 6) | | | | | | | |
| 9487 | MT- | Myoglobinuria | 9487_9501del15 | TCGCAGGATTT | FFAGFF- | − | + | Reported | 0.0% | 0 (0) | 1 |
| | CO3 | | | TTCT-del (SEQ | del | | | (alt loc) | (0.0%) | | |
| | | | | ID NO: 7) | | | | | | | |
| 9490 | MT- | Gout | C9490T | C-T | A-V | + | − | Reported | 0.0% | 22 (0) | 1 |
| | CO3 | | | | | | | | (0.0%) | | |
| 9537 | MT- | Leigh Disease | C9537CC | C-CC | frameshift | + | − | Reported | 0.0% | 0 (0) | 2 |
| | CO3 | | | | | | | | (0.0%) | | |
| 9544 | MT- | Sporadic bilateral | G9544A | G-A | G-E | · | · | Reported | 0.0% | 0 (0) | 1 |
| | CO3 | optic neuropathy | | | | | | | (0.0%) | | |
| 9559 | MT- | Rhabdomyolysis | C9559del | C-del | frameshift | − | + | Reported | 0.0% | 0 (0) | 1 |
| | CO3 | | | | | | | | (0.0%) | | |
| 9660 | MT- | LHON | A9660C | A-C | M-L | + | − | Reported | 0.0% | 0 (0) | 1 |
| | CO3 | | | | | | | | (0.0%) | | |
| 9738 | MT- | LHON | G9738T | G-T | A-S | + | − | Reported | 0.0% | 0 (0) | 1 |
| | CO3 | | | | | | | | (0.0%) | | |
| 9789 | MT- | Myopathy | T9789C | T-C | S-P | − | + | Reported | 0.0% | 0 (0) | 1 |
| | CO3 | | | | | | | | (0.0%) | | |
| 9804 | MT- | LHON | G9804A | G-A | A-T | + | − | Reported | 0.3% | 149 | 10 |
| | CO3 | | | | | | | | (0.0%) | (0) | |
| 9856 | MT- | LVNC | T9856C | T-C | I-T | + | − | Reported | 0.0% | 17 (0) | 2 |
| | CO3 | cardiomyopathy/gout | | | | | | | (0.0%) | | |
| 9861 | MT- | AD | T9861C | T-C | F-L | + | − | Reported | 0.2% | 101 | 1 |
| | CO3 | | | | | | | | (0.0%) | (0) | |
| 9952 | MT- | Mitochondrial | G9952A | G-A | W-Ter | − | + | Reported | 0.0% | 0 (0) | 1 |
| | CO3 | Encephalopathy | | | | | | | (0.0%) | | |
| 9957 | MT- | PEM/MELAS/ | T9957C | T-C | F-L | − | + | Reported | 0.1% | 41 (0) | 8 |
| | CO3 | NAION/HCM/gout | | | | | | | (0.0%) | | |
| 9966 | MT- | LHON possible | G9966A | G-A | V-I | · | · | Reported | 0.7% | 346 | 1 |
| | CO3 | helper variant | | | | | | | (0.0%) | (0) | |
| 9972 | MT- | EXIT & APS2 - | A9972C | A-C | I-L | − | + | Reported | 0.0% | 1 (0) | 1 |
| | CO3 | possible link | | | | | | | (0.0%) | | |
| 10086 | MT- | Hypertensive end- | A10086G | A-G | N-D | + | − | Reported | 0.8% | 422 | 6 |
| | ND3 | stage renal disease | | | | | | | (0.0%) | (0) | |
| 10158 | MT- | Leigh Disease/ | T10158C | T-C | S-P | + | + | Cfrm | 0.0% | 0 (0) | 27 |
| | ND3 | MELAS | | | | | | | (0.0%) | | |
| 10191 | MT- | Leigh Disease/Leigh- | T10191C | T-C | S-P | − | + | Cfrm | 0.0% | 0 (0) | 25 |
| | ND3 | like Disease/ESOC | | | | | | | (0.0%) | | |
| 10197 | MT- | Leigh Disease/ | G10197A | G-A | A-T | + | + | Cfrm | 0.0% | 4 (0) | 20 |
| | ND3 | Dystonia/Stroke/ | | | | | | | (0.0%) | | |
| | | LDYT | | | | | | | | | |
| 10237 | MT- | LHON | T10237C | T-C | I-T | + | − | Reported | 0.2% | 82 (0) | 3 |
| | ND3 | | | | | | | | (0.0%) | | |
| 10254 | MT- | Leigh Disease | G10254A | G-A | D-N | − | + | Reported | 0.0% | 0 (0) | 1 |
| | ND3 | | | | | | | | (0.0%) | | |
| 10398 | MT- | Invasive Breast Cancer | A10398A | A-A | T-T | + | − | Reported; | 55.7% | 2792 | 19 |
| | ND3 | risk factor AD PD BD | | | | | | lineage | (0.0%) | 9 (0) | |
| | | lithium response Type | | | | | | N marker | | | |
| | | 2 DM | | | | | | except | | | |
| | | | | | | | | hg IJK | | | |
| 10398 | MT- | PD protective factor/ | A10398G | A-G | T-A | + | − | Reported; | 44.3% | 2223 | 34 |
| | ND3 | longevity/altered cell | | | | | | lineage | (0.0%) | 9 (0) | |
| | | pH/metabolic | | | | | | L & M | | | |
| | | syndrome/breast | | | | | | marker, | | | |
| | | cancer risk/LS risk/ | | | | | | also hg | | | |
| | | ADHD/cognitive | | | | | | IJK | | | |
| | | decline/SCA2 age of | | | | | | | | | |
| | | onset | | | | | | | | | |
| 10543 | MT- | LHON | A10543G | A-G | H-R | − | + | Reported | 0.0% | 0 (0) | 1 |
| | ND4L | | | | | | | | (0.0%) | | |
| 10591 | MT- | LHON | T10591G | T-G | F-C | − | + | Reported | 0.0% | 0 (0) | 1 |
| | ND4L | | | | | | | | (0.0%) | | |
| 10652 | MT- | BD/MDD-associated | T10652C | T-C | I-I | − | + | Reported | 0.1% | 53 (0) | 1 |
| | ND4L | | | | | | | | (0.0%) | | |
| 10663 | MT- | LHON | T10663C | T-C | V-A | + | − | Cfrm | 0.0% | 1 (0) | 13 |
| | ND4L | | | | | | | | (0.0%) | | |
| 10680 | MT- | LHON/synergistic | G10680A | G-A | A-T | + | − | Reported/ | 0.0% | 18 (0) | 4 |
| | ND4L | combo 10680A + | | | | | | possibly | (0.0%) | | |
| | | 12033G + 14258A | | | | | | synergistic | | | |
| 11042 | MT- | Biliary atresia | T11042C | T-C | Y-H | − | + | Reported | 0.0% | 0 (0) | 1 |
| | ND4 | | | | | | | | (0.0%) | | |
| 11048 | MT- | Biliary atresia | T11048del | T-del | frameshift | − | + | Reported | 0.0% | 0 (0) | 1 |
| | ND4 | | | | | | | | (0.0%) | | |
| 11084 | MT- | AD, PD MELAS | A11084G | A-G | T-A | + | + | Conflicting | (0.0%) | 202 | 7 |
| | ND4 | | | | | | | reports | | (0) | |
| 11232 | MT- | CPEO | T11232C | T-C | L-P | − | + | Reported | 0.0% | 0 (0) | 4 |
| | ND4 | | | | | | | | (0.0%) | | |
| 11240 | MT- | Leigh Syndrome | C11240T | C-T | L-F | − | + | Reported | 0.0% | 0 (0) | 2 |
| | ND4 | | | | | | | | (0.0%) | | |
| 11251 | MT- | Reduced risk of PD | A11251G | A-G | L-L | · | · | Reported | 9.3% | 4669 | 2 |
| | ND4 | | | | | | | | (0.0%) | (0) | |
| 11253 | MT- | LHON PD | T11253C | T-C | I-T | + | − | Reported | (0.0%) | 252 | 7 |
| | ND4 | | | | | | | | | (0) | |
| 11365 | MT- | found in 1 HCM | T11365C | T-C | A-A | + | − | Reported | (0.0%) | 110 | 1 |
| | ND4 | patient | | | | | | | | (0) | |
| 11375 | MT- | found in 1 sCJD | A11375C | A-C | K-Q | + | − | Reported | 0.0% | 0 (0) | 1 |
| | ND4 | patient | | | | | | | (0.0%) | | |
| 11467 | MT- | Altered brain pH/ | A11467G | A-G | L-L | + | − | Reported | 12.4% | 6234 | 3 |
| | ND4 | sCJD patients | | | | | | | (0.0%) | (0) | |
| 11470 | MT- | MELAS | A11470C | A-C | K-N | − | + | Reported | 0.0% | 0 (0) | 1 |
| | ND4 | | | | | | | | (0.0%) | | |
| 11621 | MT- | CPEO, exercise | 11621_11622del | TA-del | frameshift | − | + | Reported | 0.0% | 0 (0) | 1 |
| | ND4 | intolerance | TA | | | | | | (0.0%) | | |
| 11696 | MT- | LHON/LDYT/ | G11696A | G-A | V-I | + | + | Reported - | 0.6% | 299 | 16 |
| | ND4 | DEAF/hypertension | | | | | | possibly | (0.0%) | (0) | |
| | | helper mut. | | | | | | synergistic | | | |
| 11777 | MT- | Leigh Disease | C11777A | C-A | R-S | − | + | Cfrm | 0.0% | 0 (0) | 12 |
| | ND4 | | | | | | | | (0.0%) | | |
| 11778 | MT- | LHON/Progressive | G11778A | G-A | R-H | + | + | Cfrm | 0.2% | 326 | 301 |
| | ND4 | Dystonia | | | | | | | (0.0%) | (0) | |
| 11832 | MT- | EXIT/oncocytoma | G11832A | G-A | W-Ter | − | + | Reported | 0.0% | 0 (0) | 6 |
| | ND4 | | | | | | | | (0.0%) | | |
| 11874 | MT- | LBON | C11874A | C-A | T-N | + | − | Reported | 0.0% | 0 (0) | 2 |
| | ND4 | | | | | | | | (0.0%) | | |
| 11919 | MT- | Thyroid Cancer Cell | C11919T | C-T | S-F | + | − | Reported | 0.0% | 0 (0) | 2 |
| | ND4 | Line | | | | | | | (0.0%) | | |
| 11984 | MT- | Leigh Syndrome | T11984C | T-C | Y-H | + | − | Reported | 0.1% | 51 (0) | 1 |
| | ND4 | | | | | | | | (0.0%) | | |
| 11994 | MT- | Oligoasthenoteratozoo | C11994T | C-T | T-I | + | − | Conflicting | 0.0% | 0 (0) | 3 |
| | ND4 | spermia (OAT) | | | | | | reports | (0.0%) | | |
| 12015 | MT- | Atypical MELAS | T12015C | T-C | L-P | − | + | Reported | 0.0% | 2 (0) | 2 |
| | ND4 | | | | | | | | (0.0%) | | |
| 12026 | MT- | DM | A12026G | A-G | I-V | + | − | Reported | 0.5% | 245 | 4 |
| | ND4 | | | | | | | | (0.0%) | (0) | |
| 12027 | MT- | SZ-associated | T12027C | T-C | I-T | · | · | Reported | 0.0% | 2 (0) | 2 |
| | ND4 | | | | | | | | (0.0%) | | |
| 12033 | MT- | LHON synergistic | A12033G | A-G | N-S | + | − | Reported: | 0.0% | 21 (0) | 1 |
| | ND4 | combo 10680A + | | | | | | individually | (0.0%) | | |
| | | 12033G + 14258A | | | | | | neutral | | | |
| | | | | | | | | variants | | | |
| | | | | | | | | causing | | | |
| | | | | | | | | LHON in | | | |
| | | | | | | | | combination | | | |
| 12338 | MT- | DEAF 1555 increased | T12338C | T-C | M-T | + | − | Conflicting | 0.3% | 174 | 11 |
| | ND5 | penetrance/LHON | | | | | | reports | (0.0%) | (0) | |
| 12361 | MT- | Non-alcoholic fatty | A12361G | A-G | T-A | + | − | Reported | 0.5% | 235 | 2 |
| | ND5 | liver disease | | | | | | | (0.0%) | (0) | |
| 12372 | MT- | Altered brain pH/ | G12372A | G-A | L-L | + | − | Reported | 13.4% | 6742 | 3 |
| | ND5 | sCJD patients | | | | | | | (0.0%) | (0) | |
| 12397 | MT- | PD, early onset | A12397G | A-G | T-A | + | − | Reported | 6.7% | 335 | 3 |
| | ND5 | | | | | | | | (0.0%) | (0) | |
| 12414 | MT- | EXIT | T12414del | T-del | frameshift | · | · | Reported | 0.0% | 0 (0) | 1 |
| | ND5 | | | | | | | | (0.0%) | | |
| 12425 | MT- | Mitochondrial | A12425del | A-del | frameshift | − | + | Reported | 0.0% | 2 (0) | 1 |
| | ND5 | Myopathy & Renal | | | | | | | (0.0%) | | |
| | | Failure | | | | | | | | | |
| 12477 | MT- | possible HCM | T12477C | T-C | S-S | + | − | Reported | 0.5% | 263 | 1 |
| | ND5 | susceptibility | | | | | | | (0.0%) | (0) | |
| 12622 | MT- | Leigh Disease | G12622A | G-A | V-I | + | + | Significance | 0.0% | (0 (0) | 2 |
| | ND5 | | | | | | | unclear | (0.0%) | | |
| 12631 | MT- | found in 2 sCJD | T12631A | T-A | S-T | + | − | Reported | 0.0% | 0 (0) | 2 |
| | ND5 | patients | | | | | | | (0.0%) | | |
| 12634 | MT- | Thyroid Cancer Cell | A12634G | A-G | I-V | + | − | Reported | 0.3% | 141 | 3 |
| | ND5 | Line | | | | | | | (0.0%) | (0) | |
| 12686 | MT- | Dilated | T12686A | T-A | F-Y | + | − | Reported | 0.0% | 0 (0) | 1 |
| | ND5 | Cardiomyopathy | | | | | | | (0.0%) | | |
| 12706 | MT- | Leigh Disease | T12706C | T-C | F-L | − | + | Cfrm | 0.0% | 0 (0) | 10 |
| | ND5 | | | | | | | | (0.0%) | | |
| 12770 | MT- | MELAS | A12770G | A-G | E-G | − | + | Reported | 0.0% | 1 (0) | 3 |
| | ND5 | | | | | | | | (0.0%) | | |
| 12778 | MT- | Dilated | G12778C | G-C | G-R | + | − | Reported | 0.0% | 0 (0) | 1 |
| | ND5 | Cardiomyopathy | | | | | | | (0.0%) | | |
| 12782 | MT- | LHON | T12782G | T-G | I-S | − | + | Reported | 0.0% | 0 (0) | 1 |
| | ND5 | | | | | | | | (0.0%) | | |
| 12811 | MT- | Possible LHON factor | T12811C | T-C | Y-H | + | − | Reported | 1.3% | 633 | 9 |
| | ND5 | | | | | | | | (0.0%) | (0) | |
| 12848 | MT- | LHON | C12848T | C-T | A-V | − | + | Reported | 0.0% | 0 (0) | 3 |
| | ND5 | | | | | | | | (0.0%) | | |
| 13042 | MT- | Optic neuropathy/ | G13042A | G-A | A-T | − | + | Cfrm | 0.0% | 1 (0) | 7 |
| | ND5 | retinopathy/LD | | | | | | | (0.0%) | | |
| 13045 | MT- | MELAS/LHON/ | A13045C | A-C | M-L | − | + | Reported | 0.0% | 0 (0) | 4 |
| | ND5 | Leigh overlap | | | | | | | (0.0%) | | |
| | | syndrome | | | | | | | | | |
| 13046 | MT- | LHON/MELAS | T13046C | T-C | M-T | − | + | Reported | 0.0% | 0 (0) | 1 |
| | ND5 | overlap syndrome | | | | | | | (0.0%) | | |
| 13051 | MT- | LHON | G13051A | G-A | G-S | + | − | Cfrm | 0.0% | 0 (0) | 2 |
| | ND5 | | | | | | | | (0.0%) | | |
| 13063 | MT- | Adult-onset | G13063A | G-A | V-I | − | + | Reported | 0.0% | 7 (0) | 3 |
| | ND5 | Encephalopathy/ | | | | | | | (0.0%) | | |
| | | Ataxia | | | | | | | | | |
| 13084 | MT- | MELAS/Leigh | A13084T | A-T | S-C | − | + | Reported | 0.0% | 0 (0) | 4 |
| | ND5 | Disease | | | | | | | (0.0%) | | |
| 13094 | MT- | Ataxia + PEO/ | T13094C | T-C | V-A | + | + | Cfrm | 0.0% | 1 (0) | 2 |
| | ND5 | MELAS, LD, LHON, | | | | | | | (0.0%) | | |
| | | myoclonus, fatigue | | | | | | | | | |
| 13135 | MT- | possible HCM | G13135A | G-A | A-T | + | − | Reported | 0.9% | 463 | 2 |
| | ND5 | susceptibility | | | | | | | (0.0%) | (0) | |
| 13204 | MT- | Peripheral neuropathy | G13204A | G-A | V-I | + | − | Reported | 0.1% | 40 (0) | 4 |
| | ND5 | of T2 diabetes | | | | | | | (0.0%) | | |
| 13271 | MT- | Exercise intolerance | T13271C | T-C | L-P | − | + | Reported | 0.0% | 1 (0) | 2 |
| | ND5 | (EXIT) | | | | | | | (0.0%) | | |
| 13276 | MT- | MIDD + retinopathy | A13276G | A-G | M-V | + | − | Conflicting | 3.3% | 1673 | 2 |
| | ND5 | | | | | | | Reports | (0.0%) | (0) | |
| 13379 | MT- | LHON | A13379C | A-C | H-P | + | − | Reported | 0.0% | 0 (0) | 1 |
| | ND5 | | | | | | | | (0.0%) | | |
| 13511 | MT- | Leigh-like syndrome | A13511T | A-T | K-M | − | + | Reported | 0.0% | 0 (0) | 3 |
| | ND5 | | | | | | | | (0.0%) | | |
| 13513 | MT- | Leigh Disease/ | G13513A | G-A | D-N | − | + | Cfrm | 0.0% | 1 (0) | 41 |
| | ND5 | MELAS/LHON- | | | | | | | (0.0%) | | |
| | | MELAS Overlap | | | | | | | | | |
| | | Syndrome/negative | | | | | | | | | |
| | | association w Carotid | | | | | | | | | |
| | | Atherosclerosis | | | | | | | | | |
| 13514 | MT- | Leigh Disease/ | A13514G | A-G | D-G | − | + | Cfrm | 0.0% | 0 (0) | 15 |
| | ND5 | MELAS/Ca2 + | | | | | | | (0.0%) | | |
| | | downregulation | | | | | | | | | |
| 13528 | MT- | LHON-like, LHON, | A13528G | A-G | T-A | + | − | Reported | 0.1% | 49 (0) | 5 |
| | ND5 | MELAS | | | | | | | (0.0%) | | |
| 13580 | MT- | Thyroid Cancer | C13580G | C-G | A-G | − | + | Reported | 0.0% | 0 (0) | 1 |
| | ND5 | | | | | | | | (0.0%) | | |
| 13637 | MT- | Possible LHON factor | A13637G | A-G | Q-R | + | − | Reported | 0.8% | 382 | 4 |
| | ND5 | | | | | | | | (0.0%) | (0) | |
| 13708 | MT- | LHON/Increased MS | G13708A | G-A | A-T | + | − | Conflicting | 7.1% | 3563 | 49 |
| | ND5 | risk/higher freq in | | | | | | reports | (0.0%) | (0) | |
| | | PD-ADS | | | | | | | | | |
| 13730 | MT- | LHON | G13730A | G-A | G-E | − | + | Reported | 0.0% | 0 (0) | 7 |
| | ND5 | | | | | | | | (0.0%) | | |
| 13831 | MT- | Thyroid Cancer Cell | C13831A | C-A | L-M | − | + | Reported | 0.0% | 3 (0) | 2 |
| | ND5 | Line | | | | | | | (0.0%) | | |
| 13849 | MT- | MELAS | A13849C | A-C | N-H | + | − | Reported - | 0.0% | 1 (0) | 2 |
| | ND5 | | | | | | | possible | (0.0%) | | |
| | | | | | | | | secondary | | | |
| 13967 | MT- | Possible LHON factor | C13967T | C-T | T-M | + | − | Reported | 0.3% | 063 | 4 |
| | ND5 | | | | | | | | (0.0%) | (0) | |
| 14063 | MT- | Potentially functional | T14063C | T-C | I-T | + | − | Reported | 0.1% | 27 (0) | 2 |
| | ND5 | variant cosegregating | | | | | | | (0.0%) | | |
| | | withLHON3635A | | | | | | | | | |
| 14091 | MT- | Developmental delay, | A14091T | A-T | K-N | − | + | Reported | 0.0% | 0 (0) | 2 |
| | ND5 | seizure, hearing loss, | | | | | | | (0.0%) | | |
| | | diabetes | | | | | | | | | |
| 14163 | MT- | Possible deafness | C14163T | C-T | A-T | + | − | Conflicting | 0.0% | 13 (0) | 3 |
| | ND6 | factor | | | | | | reports | (0.0%) | | |
| 14258 | MT- | LHON synergistic | G14258A | G-A | P-L | + | − | Reported: | 0.0% | 25 (0) | 1 |
| | ND6 | combo 10680A + | | | | | | individually | (0.0%) | | |
| | | 12033G + 14258A | | | | | | neutral | | | |
| | | also combo 14258A + | | | | | | variants | | | |
| | | 14582G | | | | | | causing | | | |
| | | | | | | | | LHON in | | | |
| | | | | | | | | combination | | | |
| 14279 | MT- | LHON | G14279A | G-A | S-L | + | − | Reported | 0.0% | 6 (0) | 3 |
| | ND6 | | | | | | | | (0.0%) | | |
| 14319 | MT- | PD, early onset | T14319C | T-C | N-D | + | − | Reported | 0.1% | 65 (0) | 3 |
| | ND6 | | | | | | | | (0.0%) | | |
| 14325 | MT- | LHON | T14325C | T-C | N-D | + | − | Reported | 0.1% | 52 (0) | 3 |
| | ND6 | | | | | | | | (0.0%) | | |
| 14340 | MT- | SNHL | C14340T | C-T | V-M | + | − | Reported | 0.0% | 23 (0) | 2 |
| | ND6 | | | | | | | | (0.0%) | | |
| 14430 | MT- | Thyroid Cancer | A14430G | A-G | W-R | + | − | Reported | 0.0% | 0 (0) | 1 |
| | ND6 | | | | | | | | (0.0%) | | |
| 14439 | MT- | Mitochondrial | G14439A | G-A | P-S | + | − | Reported | 0.0% | 0 (0) | 2 |
| | ND6 | Respiratory Chain | | | | | | | (0.0%) | | |
| | | Disorder | | | | | | | | | |
| 14441 | MT- | Leigh-like phenotype | T14441C | T-C | Y-C | · | · | Reported | 0.0% | 0 (0) | 1 |
| | ND6 | | | | | | | | (0.0%) | | |
| 14453 | MT- | MELAS/Leigh | G14453A | G-A | A-V | − | + | Reported | 0.0% | 0 (0) | 6 |
| | ND6 | Disease | | | | | | | (0.0%) | | |
| 14459 | MT- | LDYT/Leigh Disease/ | G14459A | G-A | A-V | + | + | Cfrm | 0.0% | 3 (0) | 32 |
| | ND6 | dystonia/carotid | | | | | | | (0.0%) | | |
| | | atherosclerosis risk | | | | | | | | | |
| 14482 | MT- | LHON | C14482A | C-A | M-I | + | + | Cfrm | 0.0% | 2 (0) | 13 |
| | ND6 | | | | | | | | (0.0%) | | |
| 14482 | MT- | LHON | C14482G | C-G | M-I | + | + | Cfrm | 0.0% | 0 (0) | 6 |
| | ND6 | | | | | | | | (0.0%) | | |
| 14484 | MT- | 2222203 | T14484C | T-C | M-V | + | + | Cfrm | 0.1% | 57 (0) | 170 |
| | ND6 | | | | | | | | (0.0%) | | |
| 14487 | MT- | Dystonia/Leigh | T14487C | T-C | M-V | − | + | Cfrm | 0.0% | 0 (0) | 26 |
| | ND6 | Disease/ataxia/ | | | | | | | (0.0%) | | |
| | | ptosis/epilepsy | | | | | | | | | |
| 14495 | MT- | LHON | A14495G | A-G | L-S | − | + | Cfrm | 0.0% | 2 (0) | 8 |
| | ND6 | | | | | | | | (0.0%) | | |
| 14498 | MT- | LHON | T14498C | T-C | Y-C | + | + | Reported | 0.0% | 0 (0) | 4 |
| | ND6 | | | | | | | | (0.0%) | | |
| 14502 | MT- | LHON | T14502C | T-C | I-V | + | − | Reported - | 0.4% | 186 | 7 |
| | ND6 | | | | | | | possibly | (0.0%) | (0) | |
| | | | | | | | | synergistic | | | |
| 14536 | MT- | DMDF | C14535CC | C-CC | frameshift | · | · | Reported | 0.0% | 0 (0) | 1 |
| | ND6 | | | | | | | | (0.0%) | | |
| 14568 | MT- | LHON | C14568T | C-T | G-S | + | − | Cfrm | 0.0% | 6 (0) | 10 |
| | ND6 | | | | | | | | (0.0%) | | |
| 14577 | MT- | MIDM | T14577C | T-C | I-V | − | + | Reported | 0.8% | 411 | 1 |
| | ND6 | | | | | | | | (0.0%) | (0) | |
| 14582 | MT- | LHON synergistic | A14582G | A-G | V-A | + | − | Reported: | 0.5% | 252 | 1 |
| | ND6 | combo 14258A + | | | | | | individually | (0.0%) | (0) | |
| | | 14582G | | | | | | neutral | | | |
| | | | | | | | | variants | | | |
| | | | | | | | | causing | | | |
| | | | | | | | | LHON in | | | |
| | | | | | | | | combination | | | |
| 14596 | MT- | LHON | A14596T | A-T | I-M | + | − | Reported | 0.0% | 0 (0) | 5 |
| | ND6 | | | | | | | | (0.0%) | | |
| 14600 | MT- | Leigh Disease w/optic | G14600A | G-A | P-L | + | + | Reported | 0.0% | 0 (0) | 3 |
| | ND6 | atrophy | | | | | | | (0.0%) | | |
| 14668 | MT- | Depressive Disorder | C14668T | C-T | M-M | + | − | Reported | 4.1% | 2059 | 1 |
| | ND6 | associated | | | | | | | (0.0%) | (0) | |
| 14787 | MT- | PD/MELAS | 14787_14790del | TTAA-del | frameshift | − | + | Reported | 0.0% | 0 (0) | 1 |
| | CYB | | TTAA | | | | | | (0.0%) | | |
| 14831 | MT- | LHON | G14831A | G-A | A-T | + | − | Reported | 0.2% | 104 | 2 |
| | CYB | | | | | | | | (0.0%) | (0) | |
| 14841 | MT- | LHON helper mut. | A14841G | A-G | N-S | − | + | Reported | 0.0% | 21 (0) | 1 |
| | CYB | | | | | | | | (0.0%) | | |
| 14846 | MT- | EXIT/possibly | G14846A | G-A | G-S | − | + | Reported | 0.0% | 0 (0) | 9 |
| | CYB | antiatherogenic, poss. | | | | | | | (0.0%) | | |
| | | myocardial infarction | | | | | | | | | |
| | | association | | | | | | | | | |
| 14849 | MT- | EXIT/Septo-Optic | T14849C | T-C | S-P | − | + | Cfrm | 0.0% | 0 (0) | 3 |
| | CYB | Dysplasia | | | | | | | (0.0%) | | |
| 14864 | MT- | MELAS | T14864C | T-C | C-R | − | + | Cfrm | 0.0% | 2 (0) | 1 |
| | CYB | | | | | | | | (0.0%) | | |
| 14894 | MT- | LHON | T14894C | T-C | F-L | · | · | Reported | 0.0% | 8 (0) | 1 |
| | CYB | | | | | | | | (0.0%) | | |
| 15024 | MT- | Possible DEAF | G15024A | G-A | C-Y | + | − | Reported | 0.1% | 32 (0) | 1 |
| | CYB | modifier | | | | | | | (0.0%) | | |
| 15043 | MT- | MDD-associated | G15043A | G-A | G-G | + | − | Reported | (0.0%) | 1183 | 2 |
| | CYB | | | | | | | | | 7 (0) | |
| 15059 | MT- | MM/carotid | G15059A | G-A | G-Ter | − | + | Reported | 0.0% | 0 (0) | 2 |
| | CYB | atherosclerosis risk/ | | | | | | | (0.0%) | | |
| | | essential hypertension | | | | | | | | | |
| 15077 | MT- | DEAF | G15077A | G-A | E-K | + | − | Reported | 0.2% | 102 | 2 |
| | CYB | | | | | | | | (0.0%) | (0) | |
| 15084 | MT- | EXIT | G15084A | G-A | W-Ter | − | + | Reported | 0.0% | 0 (0) | 2 |
| | CYB | | | | | | | | (0.0%) | | |
| 15092 | MT- | MELAS | G15092A | G-A | G-S | − | + | Reported | 0.0% | 0 (0) | 1 |
| | CYB | | | | | | | | (0.0%) | | |
| 15150 | MT- | EXIT | G15150A | G-A | W-Ter | − | + | Reported | 0.0% | 0 (0) | 1 |
| | CYB | | | | | | | | (0.0%) | | |
| 15153 | MT- | Suspected mito disease | G15153A | G-A | G-D | − | + | Reported | 0.0% | 6 (0) | 1 |
| | CYB | | | | | | | | (0.0%) | | |
| 15158 | MT- | Suspected mito disease | A15158G | A-G | M-V | − | + | Reported | 0.0% | 0 (0) | 1 |
| | CYB | | | | | | | | (0.0%) | | |
| 15168 | MT- | EXIT | G15168A | G-A | W-Ter | − | + | Reported | 0.0% | 0 (0) | 2 |
| | CYB | | | | | | | | (0.0%) | | |
| 15170 | MT- | EXIT | G15170A | G-A | G-Ter | − | + | Reported | 0.0% | 0 (0) | 1 |
| | CYB | | | | | | | | (0.0%) | | |
| 15197 | MT- | EXIT | T15197C | T-C | S-P | − | + | Reported | 0.0% | 0 (0) | 2 |
| | CYB | | | | | | | | (0.0%) | | |
| 15209 | MT- | Prader-Willi syndrome | T15209C | T-C | Y-H | + | − | Reported | 0.0% | 4 (0) | 1 |
| | CYB | | | | | | | | (0.0%) | | |
| 15234 | MT- | Leigh stroke-like | G15234A | G-A | W-Ter | · | · | Reported | 0.0% | 0 (0) | 1 |
| | CYB | leukodystrophy | | | | | | | (0.0%) | | |
| 15237 | MT- | Potentially functional | T15237C | T-C | I-T | + | − | Reported | 0.0% | 6 (0) | 1 |
| | CYB | variant cosegregating | | | | | | | (0.0%) | | |
| | | with LHON3635A | | | | | | | | | |
| 15242 | MT- | Mitochondrial | G15242A | G-A | G-Ter | − | + | Reported | 0.0% | 0 (0) | 2 |
| | CYB | Encephalomyopathy | | | | | | | (0.0%) | | |
| 15243 | MT- | HCM | G15243A | G-A | G-E | − | + | Reported | 0.0% | 0 (0) | 2 |
| | CYB | | | | | | | | (0.0%) | | |
| 15256 | MT- | Peripheral neuropathy | A15256G | A-G | V-V | + | − | Reported | 0.0% | 4 (0) | 1 |
| | CYB | of T2 diabetes | | | | | | | (0.0%) | | |
| 15257 | MT- | LHON | G15257A | G-A | D-N | + | − | Conflicting | (0.0%) | 763 | 45 |
| | CYB | | | | | | | reports | [symbol] | (0) | |
| | | | | | | | | | (0.0%) | | |
| 15287 | MT- | Possible DEAF | T15287C | T-C | F-L | − | + | Reported; | 0.2% | 80 (0) | 1 |
| | CYB | helper mut. | | | | | | hg I6a | (0.0%) | | |
| | | | | | | | | & H10c | | | |
| | | | | | | | | marker | | | |
| 15395 | MT- | Possible LHON factor | A15395G | A-G | K-E | + | − | Reported | 0.0% | 2 (0) | 1 |
| | CYB | | | | | | | | (0.0%) | | |
| 15453 | MT- | Isolated complex III | T15453C | T-C | L-P | + | − | Reported | 0.0% | 10 (0) | 1 |
| | CYB | deficiency | | | | | | | (0.0%) | | |
| 15497 | MT- | EXIT/Obesity | G15497A | G-A | G-S | + | − | Reported | 0.4% | 217 | 5 |
| | CYB | | | | | | | | (0.0%) | (0) | |
| 15498 | MT- | EXIT | 15498_15521del | 24bp_deletion | GDPDNY | − | + | Reported | 0.0% | 0 (0) | 2 |
| | CYB | | 24 | | TL-del | | | | (0.0%) | | |
| 15498 | MT- | DEAF/Infantile | G15498A | G-A | G-D | − | + | Reported | 0.0% | 13 (0) | 2 |
| | CYB | histiocytoid | | | | | | | (0.0%) | | |
| | | cardiomyopathy | | | | | | | | | |
| 15579 | MT- | Multisystem Disorder, | A15579G | A-G | Y-C | − | + | Cfrm | 0.0% | 0 (0) | 4 |
| | CYB | EXIT | | | | | | | (0.0%) | | |
| 15615 | MT- | EXIT/Antimycin | G15615A | G-A | G-D | − | + | Reported | 0.0% | 0 (0) | 3 |
| | CYB | resistance | | | | | | | (0.0%) | | |
| 15620 | MT- | Leigh Syndrome | C15620A | C-A | L-I | − | + | Reported | 0.0% | 0 (0) | 1 |
| | CYB | helper mut | | | | | | | (0.0%) | | |
| 15635 | MT- | Polyvisceral failure | T15635C | T-C | S-P | + | − | Reported | 0.0% | 2 (0) | 1 |
| | CYB | | | | | | | | (0.0%) | | |
| 15649 | MT- | Multisystem Disorder, | 15649_15666del | 18bp_deletion | ILAMIP- | − | + | Reported | 0.0% | 0 (0) | 1 |
| | CYB | EXIT | 18 | | del | | | | (0.0%) | | |
| 15662 | MT- | Complex | A15662G | A-G | I-V | + | + | Reported | 0.4% | 188 | 1 |
| | CYB | mitochondriopathy- | | | | | | | (0.0%) | (0) | |
| | | associated | | | | | | | | | |
| 15674 | MT- | LHON | T15674C | T-C | S-P | + | − | Reported | 0.3% | 146 | 2 |
| | CYB | | | | | | | | (0.0%) | (0) | |
| 15693 | MT- | Possibly LVNC | T15693C | T-C | M-T | + | − | Reported | 4.2% | 589 | 1 |
| | CYB | cardiomyopathy- | | | | | | | (0.0%) | (0) | |
| | | associated | | | | | | | | | |
| 15699 | MT- | Muscle Weakness | G15699C | G-C | R-P | − | + | Reported | 0.0% | 0 (0) | 2 |
| | CYB | SNHL and Migraine | | | | | | | (0.0%) | | |
| 15723 | MT- | EXIT | G15723A | G-A | W-Ter | − | + | Reported | 0.0% | 0 (0) | 1 |
| | CYB | | | | | | | | (0.0%) | | |
| 15761 | MT- | MM | G15761A | G-A | G-Ter | | + | Reported | 0.0% | 0 (0) | 1 |
| | CYB | | | | | | | | (0.0%) | | |
| 15762 | MT- | MM | G15762A | G-A | G-E | − | + | Reported | 0.0% | 0 (0) | 1 |
| | CYB | | | | | | | | (0.0%) | | |
| 15773 | MT- | LHON | G15773A | G-A | V-M | + | − | Possibly | 0.1% | 59 (0) | 1 |
| | CYB | | | | | | | synergistic | (0.0%) | | |
| 15784 | MT- | POAG - potential for | T15784C | T-C | P-P | + | − | Reported | 3.5% | 1756 | 3 |
| | CYB | association | | | | | | | (0.0%) | (0) | |
| 15800 | MT- | EXIT/Myopathy | C15800T | C-T | Q-Ter | − | + | Reported | 0.0% | 0 (0) | 2 |
| | CYB | | | | | | | | (0.0%) | | |
| 15804 | MT- | Fibromyalgia | T15804C | T-C | V-A | + | − | Reported | 0.1% | 27 (0) | 1 |
| | CYB | | | | | | | | (0.0%) | | |
| 15812 | MT- | LHON | G15812A | G-A | V-M | + | − | Reported/ | 0.9% | 466 | 20 |
| | CYB | | | | | | | Secondary | (0.0%) | (0) | |
| 16081 | MT- | Cyclic Vomiting | A16081G | A-G | noncoding | − | + | Reported | 0.0% | 1 (31) | 1 |
| | CR | Syndrome | | | | | | | (0.0%) | | |
| 16093 | MT- | Cyclic Vomiting | T16093C | T-C | noncoding | − | + | Reported | 5.7% | 2869 | 2 |
| | CR | Syndrome | | | | | | | (0.4%) | (4721) | |
| 16129 | MT- | Cyclic Vomiting | G16129A | G-A | noncoding | − | + | Reported | 13.2% | 6605 | 1 |
| | CR | Syndrome with | | | | | | | (15.7%) | (11486) | |
| | | Migraine | | | | | | | | | |
| 16176 | MT- | Cyclic Vomiting | C16176T | C-T | noncoding | − | + | Reported | 0.6% | 303 | 1 |
| | CR | Syndrome with | | | | | | | (0.8%) | (337) | |
| | | Migraine | | | | | | | | | |
| 16183 | MT- | Melanoma patients | A16183C | A-C | noncoding | · | · | Reported | 13.6% | 0812 | 1 |
| | CR | | | | | | | | (15.2%) | (11124) | |
| 16189 | MT- | Diabetes/ | T16189C | T-C | noncoding | + | − | Reported | 25.95 | 1297 | 34 |
| | CR | Cardiomyopathy/ | | | | | | | (26.1%) | 9 | |
| | | cancer risk/mtDNA | | | | | | | | (19118) | |
| | | copy nbr/Metabolic | | | | | | | | | |
| | | Syndrome/Melanoma | | | | | | | | | |
| | | patients | | | | | | | | | |
| 16192 | MT- | Melanoma patients | C16192T | C-T | noncoding | · | · | Reported | 4.2% | 2699 | 1 |
| | CR | | | | | | | | (4.3%) | (3183) | |
| 16217 | MT- | Endometriosis | T16217C | T-C | noncoding | + | − | Reported | 7.3% | 3659 | 1 |
| | CR | | | | | | | | (6.5%) | (4250) | |
| 16270 | MT- | Melanoma patients | C16270T | C-T | noncoding | · | · | Reported | 4.6% | 2317 | 1 |
| | CR | | | | | | | | (3.2%) | (2348) | |
| 16300 | MT- | BD-associated | A16300G | A-G | noncoding | + | − | Reported | 0.6% | 261 | 2 |
| | CR | | | | | | | | (0.2%) | (491) | |
| 16318 | MT- | Non-alcoholic | A16318C | A-C | noncoding | · | · | Reported | 0.2% | 94 | 1 |
| | CR | steatohepatitis - | | | | | | | (0.1%) | (069) | |
| | | potential for | | | | | | | | | |
| | | association | | | | | | | | | |
| 16390 | MT- | POAG - potential for | G16390A | G-A | noncoding | + | − | Reported | 5.9% | 2947 | 3 |
| | CR | association | | | | | | | (6.1%) | (4159) | |
| 16519 | MT- | Cyclic Vomiting | T16519T | T-T | noncoding | + | − | Reported | 36.9% | 1853 | 4 |
| | CR | Syndrome with | | | | | | | (0.0%) | 1 (0) | |
| | | Migraine/metastasis | | | | | | | | | |

Column Heading Key: A: Position; B: Locus; C: Disease; D: Allele; E: RNA; F: Homoplasmy; G: Heteroplasmy; H: Status; I: MitoTip; J: GB Freq FL (CR); K: GB Seqs FL (CR); L: Reference

TABLE 4
“Top 19” Primary Leber's Hereditary Optic Neuropathy (LHON) mutations,
the first 3 mutations listed (in boldface) represent approximately 95% of all cases. The
remaining mutations are listed in nucleotide order.

| | | | AA | % | % | | Penetrance [symbol] | Penetrance [symbol] | % | |
|---|---|---|---|---|---|---|---|---|---|---|
| Mutation | NT Δ | AA Δ | Cons [symbol] | Patients | Controls | Het. [symbol] | % Relatives | % Males | Recovery^{d} | Refs. |
| m.11778G>A | G-A | R340H | 100% | 69 | 0 | +/− | 33-60 | 82 | 4 | (27) |
| ND4 | | | | | | | | | | |
| m.3460G>A | G-A | A52T | 91% | 13 | 0 | +/− | 14-75 | 40-80 | 22 | (10, 16) |
| ND1 | | | | | | | | | | |
| m.14484T>C | T-C | M64V | 31% | 14 | 0 | +/− | 27-80 | 68 | 37-65 | (2, 13, |
| ND6 | | | | | | | | | | 18) |
| m.3376G>A | G-A | E24K | 98% | Rare | 0 | +/+ | NA | NA | NA | (35, 34, |
| ND1 | | | | | | | | | | 35) |
| m.3635G>A | G-A | SI ION | 93% | Rare | 0 | +/− | 29 | 54 | Low | (3) |
| ND1 | | | | | | | (range 11-64) | (range 25- | | |
| | | | | | | | | 100) | | |
| m.3697G>A | G-A | G131S | 100% | Rare | 0 | +/+ | NA | NA | NA | (32) |
| ND1 | | | | | | | | | | |
| m.3700G>A | G-A | A112T | 93% | Rare | 0 | − | NA | NA | UN | (1a, 7) |
| ND1 | | | | | | | | | | |
| m.3733G>A | G-A | E143K | 100% | Rare | 0 | +/− | 24-30 | 36-44 | Yes | (1a, 26) |
| ND1 | | | | | | | | | | |
| m.4171C>A | C-A | L289M | 93% | Rare | 0 | +/− | 46 | 47 | Yes | (20) |
| ND1 | | | | | | | | | | |
| m,10197G>A | G-A | A47T | 96% | Rare | 4/42616 | +/+ | NA | NA | NA | (36) |
| ND3 | | | | | | | | | | |
| m.10663T>C | T-C | V65A | 89% | Rare | 0 | +/− | 56 | 60 | UN | (1a, 1b) |
| ND4L | | | | | | | | | | |
| m.13051G>A | G-A | G239S | 98% | Rare | 0 | − | 56 | 63 | UN | (5b, 14) |
| ND5 | | | | | | | | | | |
| m.13094T>C | T-C | V253A | 100% | Rare | 0 | + | NA | NA | Yes | (5c, 23 |
| ND5 | | | | | | | | | | b) |
| m.14459G>A | G-A | A72V | 89% | Rare | 0 | + | NA | NA | Low | (3, 19, |
| ND6 | | | | | | | | | | 24) |
| m.14482C>A | C-A | M64I | 31% | Rare | 0 | +/− | NA | 89 | Yes | (1a, 25) |
| ND6 | | | | | | | | | | |
| m.14482C>G | C-G | M64I | 31% | Rare | 0 | − | NA | NA | UN | (11) |
| ND6 | | | | | | | | | | |
| m.14495A>G | A-G | L60S | 100% | Rare | 0 | + | NA | NA | Low | (4) |
| ND6 | | | | | | | | | | |
| m.14502T>C | T-C | 158V | 78% | Rare | 0 | − | 14502:10% | 14502:11% | UN | (1a, 30, |
| ND6 | | | | | | | 14502 + | 14502 + | | 31) |
| | | | | | | | 11778:37% | 11778:47% | | |
| m.14568C>T | C-T | G36S | 87% | Rare | 0 | − | NA | NA | UN | (6, 28) |
| ND6 | | | | | | | | | | |

^{a}Conservation calculated using Mitomaster with the species set shown here
^{b}Het. = Heteroplasmy; + = detected, − = not detected.
^{c}NA = not applicable; UN = unknown; penetrance values are rough estimates.
^{d}Low = anecdotal low degree of vision recovery; Yes = anecdotal moderate to high degree of vision recovery; UN = unknown; NA = not applicable [symbol]
indicates data missing or illegible when filed

TABLE 5
Other candidate LHON mutations found as single family or singleton cases.

| | | | AA | # | | | | |
|---|---|---|---|---|---|---|---|---|
| Mutation | NT Δ | AA Δ | Cons [symbol] | Patients | # Controls | Het. [symbol] | Recovery [symbol] | References |
| m.3472T>C | T-C | F56L | 96% | 1 case | 3 | − | UN | (22b) |
| ND1 | | | | | | | | |
| m.4025C>T | C-T | T240M | 33% | 1 family; 3 cases | 0 | − | UN | (15) |
| ND1 | | | | | | | | |
| m.4160T>C | T-C | L285P | 100% | 1 family; 9 cases | 1 | − | UN | (13) |
| ND1 | | | | | | | | |
| m.4640C>A | C-A | I57M | 27% | 1 family; 4 cases | 0 | − | UN | (3) |
| ND2 | | | | | | | | |
| m.5244G>A | G-A | G259S | 100% | 1 case | 0 | + | UN | (1b) |
| ND2 | | | | | | | | |
| m.9101T>C | T-C | I192T | 13% | 1 case | 0 | − | UN | (21) |
| ATP6 | | | | | | | | |
| m.9804G>A | G-A | A200T | 93% | Multiple unrelated | 0 | − | UN | (14, 17) |
| CO3 | | | | singleton cases | | | | |
| m,10237T>C | T-C | I60T | 100% | 1 family; 2 cases | 0 | − | UN | (9) |
| ND3 | | | | | | | | |
| m,11253T>C | T-C | I165T | 42% | 1 case | 0 | − | Yes | (22) |
| ND4 | | | | | | | | |
| m.11696G>A (ND4) & | G-A | V312I | 7% | 1 family; 11 cases | 0 | + | UN | (5) |
| m.14596A>T (ND6) | A-T | I26M | 84% | | | | | |
| m.12811T>C | T-C | Y159H | 56% | 1 family; 2 cases | 0 | − | UN | (15) |
| ND5 | | | | | | | | |
| m.12848C>T | C-T | A171V | 98% | 1 case | 0 | + | UN | (23) |
| ND5 | | | | | | | | |
| m.13637A>G | A-G | Q434R | 62% | 1 family; 3 cases | 0 | − | UN | (15) |
| ND5 | | | | | | | | |
| m.13730G>A | G-A | G465E | 100% | 1 case | 0 | + | Yes | (12) |
| ND5 | | | | | | | | |
| m.14279G>A | G-A | S132L | 47% | 1 family; 2 cases | 0 | − | UN | (29) |
| ND6 | | | | | | | | |
| m.14325T>C | T-C | N117D | 18% | 1 case | 0 | − | UN | (14) |
| ND6 | | | | | | | | |
| m.14498T>C | T-C | Y59C | 98% | 1 case | 0 | +/− | UN | (28) |
| ND6 | | | | | | | | |
| m.14831G>A | G-A | A29T | 42% | 1 case | 0 | − | UN | (7) |
| CytB | | | | | | | | |

^{a}Conservation calculated using Mitomaster with the species set shown here
^{b}Het. = Heteroplasmy; + = detected, − = not detected.
^{c}NA = not applicable; UN = unknown; penetrance values are rough estimates.
^{d}Low = anecdotal low degree of vision recovery; Yes = anecdotal moderate to high degree of vision recovery; UN = unknown; NA = not applicable [symbol]
indicates data missing or illegible when filed

Other databases and/or tools that can be used to identify and/or characterize a mtDNA mutation in a mtDNA sequence can include PhyloTree (www.phylotree.org), Haplogrep (https://haplogrep.i-med.ac.at), MSeqDR (https://mseqdr.org/MITO/genes), AmtDB (https://amtdb.org), HmtDB (https://www.hmtdb.uniba.it), PON tRNA (http://structure.bmc.lu.se/PON-mt-tRNA/), MitImpact (http://mitimpact.css-mendel.it), HvrBase++ (http://hyrbase.cibiv.univie.ac.at), GiiB-JST mtSNP (http://mtsnp.tmig.orjp/mtsnp/index_e.shtml), HmtVar (https://www.hmtvar.uniba.it), mt-DNA Server (https://mtdna-server.uibk.ac.at/index.html), EMPOP CR (empop.online), Mitominer (http://mitominer.mrc-mbu.cam.ac.uk/release-4.0/begin.do), POLG Pathogenicity Server (https://www.mitomap.org/polg/), MitoWheel (https://www.mitomap.org/MITOMAP), POLG @NIEHS (https://tools.niehs.nih.gov//polg/), MitoBreak (http://mitobreak.portugene.com/cgi-bin/Mitobreak_home.cgi), MitoAge (http://www.mitoage.info), Mamit-tRNA/mitotRNAdb (http://mttrna.bioinf.uni-leipzig.de/mtDataOutput/), MitoFit (https://www.mitofit.org/index.php/MitoFit), Misynpat (http://misynpat.org/misynpat/).

### Cells and Cell Populations

In some embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof. In some embodiments, the cell or cell population can be or include one or more circulating mononuclear cell(s) and wherein the cell signature comprises a circulating mononuclear cell signature. In some embodiments, the one or more circulating mononuclear cells comprise lymphocyte(s), monocyte(s), dendritic cell(s) or a combination thereof. In some embodiments, the one or more cells can be or include one or more peripheral blood mononuclear cells. In some embodiments, the one or more cells can be an immune cell. In some embodiments, the one or more circulating mononuclear cells comprise T cell(s), B cell(s), natural killer cell(s) or a combination thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, CD 25+, effector Th, memory Th, regulatory Th, CD4+/CD8+ thymocytes, CD4—/CD8— thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used herein, “B cell” refers to any number of a diverse population of similar types of white blood cell. B cells may be recognised, for example, by function, by phenotype and/or by gene expression pattern, particularly by cell surface phenotype. B cells can be professional antigen presenting cells, which can express both MHC I and MHC II molecules. B cells can also be identified by the expression of a Pre-B cell Receptor or a B cell receptor. In some embodiments, the B cell expresses a B cell receptor. In some embodiments, a B cell can be identified by its ability to secrete antibodies.

As used throughout this specification “macrophage” refers to a heterogenous population of leukocytes specialized and capable of detecting, phagocytosing, attacking, and/or destroying bacteria and other harmful organisms, pathogens, and other cells that can be differentiated from monocytes. Macrophages can be professional antigen presenting cells and can express MHC I and MHC II molecules. Macrophages can release cytokines and thus can stimulate inflammatory processes in other cells. Macrophages can express pathogen recognition molecules such as Toll-like receptors, which can bind specifically to different pathogenic and non-pathogenic components, such as sugars (e.g. lipopolysaccharide), RNA, DNA, and extracellular proteins and peptides. Macrophages exist in nearly all tissues and are differentiated from monocytes. The type of macrophage depends upon the type(s) of cytokines that the monocytes are exposed to during differentiation. Both macrophages and monocytes (specifically defined elsewhere herein) can both non-specific defense (innate immunity) as well as to help initiate specific defense mechanisms (adaptive immunity) of vertebrates. They also can stimulate lymphocytes and other immune cells to respond to pathogens.

As used throughout this specification, “monocyte” may refer to a type of white blood cells capable of dividing and differentiating into and hence replenishing or producing macrophages and dendritic cells, e.g., under normal states or in response to inflammation signals. Monocytes are typically identified in stained smears by their large bilobate nucleus. Monocytes are further typified by expression of CD14 and can also show expression of one or more of following surface markers such as 1251-WVH-1, Adipophilin, CB12, CD11a, CD11b, CD15, CD54, CD163, cytidine deaminase, or FLT1. Monocytes encompass previously known subtypes, such as the ‘classical’ monocyte, the ‘non-classical’ monocyte and the ‘intermediate’ monocyte, which are present in human tissues such as blood. ‘Classical’ monocytes are typified by high level expression of CD14 (CD14⁺⁺ monocyte) and ‘non-classical’ monocytes display low level expression of CD14 and additional co-expression of CD16 (CD14⁺CD16⁺⁺ monocyte). ‘Intermediate’ monocytes show a phenotype intermediate between the aforementioned types in terms of CD14 and CD16 expression (CD14⁺⁺CD16⁺ monocyte).

As used herein, “T cell” refers to a lymphocyte produced and/or processed by the thymus gland and can actively participate in the immune response. T cells can include ithymocytes, Th or Tc; Th1, Th2, Th17, Th9, Tfh, Thαβ, CD4+, CD8+, CD 25+, effector Th, memory Th, regulatory Th, CD4+/CD8+ thymocytes, CD4−/CD8− thymocytes, γδ T cells, natural killer T cells, etc. T cells can express a T cell receptor.

As used herein, “circulating mononuclear cells” refers to a mononuclear cell that can be found in the bloodstream, lymph, and/or cerebrospinal fluid. “Circulating mononuclear cells” include peripheral blood mononuclear cells. peripheral blood mononuclear cells include any peripheral blood cell having a round nucleus. Peripheral blood mononuclear cells include, for example, T cells, B cells, and natural killer cells.

Samples

In some embodiments, the sample is a bodily fluid, a bodily excretion, a bodily secretion, bodily excretion, a tissue, a cell or cell population, or a combination thereof. In some embodiments, the sample has one or more mitochondria. Bodily fluids include, but are not limited to, blood, saliva, semen, vaginal fluids, mucus, urine, breast milk, sweat, tears and otic fluids, cerebrospinal fluid, lymph, gastric juices, synovial fluid, pleural fluid, pericardial fluid, peritoneal fluid, amniotic fluid, combinations thereof, and components thereof. As used herein, “bodily secretions” refers to endogenous substances produced through the activity of cells, glands, tissues, organs, and/or organ systems. As used herein, “bodily excretion” refers to any product from a cell, gland, tissue, organ, and/or organ system that is eliminated from the body. In some embodiments, the sample is blood or component thereof. The sample can be processed, preserved, and/or otherwise prepared for analysis by one or more of the methods described herein by any suitable method.

### Methods of Detecting Mitochondrial Diseases and Uses Thereof

Also described herein are methods of detecting mitochondrial diseases. As used herein “mitochondrial diseases” refers to any disease, disorder, syndrome, condition, or a symptom thereof that is caused, directly or indirectly, by mitochondrial dysfunction. In some embodiments, the mitochondrial dysfunction can be caused, in part or in whole, by one or more mtDNA mutations. In some embodiments, the one or more mtDNA mutations can be one or more mutations set forth in any one or more of Tables 1-5. In some embodiments, the mitochondrial disease is any disease set forth in any one or more of Tables 1-5.

In some embodiments, detecting a mitochondrial disease can include detecting mitochondrial DNA (mtDNA) heteroplasmy and cell type and/or cell state in a cell or cell population, wherein detecting includes detecting, in a sample comprising the cell or cell population, a cell signature in the cell or cell population, and detecting mtDNA heteroplasmy in the cell or cell population, where the cell signature and/or mtDNA heteroplasmy indicates at least a cell type and/or a cell state.

Methods of Diagnosing, Prognosing, and/or Monitoring Mitochondrial Diseases.

Detection of mitochondrial diseases can be used to diagnose, prognose, and/or monitor diseases. Also described herein are methods of diagnosing, prognosing a mitochondrial disease.

In some embodiments, methods of diagnosing, prognosing, and/or monitoring a mitochondrial disease can include detecting mitochondrial DNA (mtDNA) heteroplasmy and cell type and/or cell state in a cell or cell population, wherein detecting can include detecting, in a sample comprising the cell or cell population, a cell signature in the cell or cell population, and detecting mtDNA heteroplasmy in the cell or cell population, where the cell signature and/or mtDNA heteroplasmy indicates at least cell type and/or cell state; and optionally repeating detecting mtDNA heteroplasmy and cell type and/or cell state one or more times over a period of time. In some embodiments, detecting mtDNA heteroplasmy can be repeated 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 times or more. In some embodiments, the period of time can range from 1 to 10 minutes, days, weeks, months, or years, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 minutes, days, weeks, months, or years.

As used herein, “diagnosing” encompasses detecting, analyzing, measuring, and/or determining the existence, nature, stage, and/or characteristic of a disease, disorder, condition, syndrome, or a symptom thereof in a subject. As understood by those skilled in the art, a diagnosis does not necessarily indicate that it is certain that a subject certainly has the disease, but rather that it is very likely that the subject has the disease. It will be appreciated that in some cases, the diagnosis is a certainty that a subject has a particular disease, disorder, condition, syndrome, or a symptom thereof. A diagnosis can be provided with varying levels of certainty, such as indicating that the presence of the disease is 90% likely, 95% likely, 98%, 99%, or 100% likely, for example. The term diagnosis, as used herein also encompasses determining the severity and probable outcome of disease or episode of disease or prospect of recovery, which is generally referred to as prognosis. The term diagnosis, as used herein, also encompasses determining a stage and/or other characteristic of a disease.

As used herein, “prognosis”, “prognose”, or “prognosing” refer to a prediction of a probability, course, or outcome. Specifically, “prognosing an mitochondrial disease” refers to the prediction that a subject has a mitochondrial disease or a symptom thereof or that a subject will develop a mitochondrial disease or a symptom thereof. For example, the prognostic methods of the instant invention provide for determining whether a subject exhibits specific characteristics (e.g. a specific signature, such as any of those described herein, mtDNA heteroplasmy, mtDNA mutation, or any combination thereof), which can be used to predict whether a subject in need thereof has or will develop a mitochondrial disease or a symptom thereof. The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity, genotype, mtDNA heteroplasmy, or phenotype of the cells and/or mitochondria in the subject compared to a control subject having normal mitochondria status or not having a disease comprising a mtDNA or mtDNA heterplasmy component indicates that the subject has an impaired mitochondria status and/or has a disease comprising an mtDNA, mitochondria dysfunction, and/or mtDNA heteroplasmy component or would benefit from a therapy targeting the mitochondria, cell, mtDNA mutation, or a combination thereof.

Hence, the methods may rely on comparing the quantity, quality, sequence, heteroplasmy, of cells, mitochondria, mtDNA, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

As used herein, “monitoring” refers to evaluating the development (or non-development) and/or progression (or non-progression or regression) of a disease or a symptom thereof or an indicator (e.g., a biomarker, signature, and the like) in a subject over a period of time.

In some embodiments, the cell signature comprises a chromatin accessibility signature, a gene expression signature, a protein expression signature, an epigenetic state signature, a cell surface marker expression signature, a cell activity signature, a phenotypic profile, a cell landscape, or a combination thereof. Signatures are discussed in greater detail elsewhere herein.

In some embodiments, detecting the signature and/or detecting mtDNA heteroplasmy is/are determined by a sequencing method. Suitable sequencing methods are described in greater detail elsewhere herein. In some embodiments, the sequencing method includes or is single cell RNA sequencing and/or mitochondrial DNA single cell ATAC-seq (mtscATAC-seq).

In some embodiments, detecting a cell signature comprises measuring a change in a distance in gene expression or accessible fragment space between two or more cell states. In some embodiments, the gene expression and/or accessible fragment space comprises 1 to 1000 or more accessible genes and/or accessible fragments, such as 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 or more genes and/or accessible fragments. In some embodiments, the gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments. In some embodiments, the distance in gene expression and/or accessible fragment space is measured by a Euclidean distance, Pearson coefficient, Spearman coefficient, or combination thereof.

In some embodiments, detecting mtDNA heteroplasmy comprises detecting one or more mutations in the mtDNA. In some embodiments, at least one of the one or more mutations are pathogenic. In some embodiments, the at least one of the one or more mtDNA mutations is selected from the group of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), one or more mutations as set forth in any one or more of Tables 1-5, or any combination thereof.

In some embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof. In some embodiments, the cell or cell population comprises one or more circulating mononuclear cell(s) and the cell signature comprises a circulating mononuclear cell signature. In some embodiments, the one or more circulating mononuclear cells comprise one or more peripheral blood mononuclear cells. In some embodiments, the one or more circulating mononuclear cells comprise lymphocyte(s), monocyte(s), dendritic cell(s) or a combination thereof. In some embodiments, the one or more circulating mononuclear cells comprise T cell(s), B cell(s), natural killer cell(s) or a combination thereof.

In some embodiments, the sample is a bodily fluid, a bodily excretion, a bodily secretion, a tissue, a cell or cells, or a combination thereof. In some embodiments, the sample is blood.

In some embodiments, the mitochondrial disease is a maternally inherited mitochondrial disease. In some embodiments, the mitochondrial disease is a heteroplasmic mitochondrial disease. In some embodiments, the mitochondrial disease is MELAS (mitochondrial myopathy encephalopathy, and lactic acidosis and stroke-like episodes), CPEO/PEO (chronic progressive external opthalmoplegia syndrome/progressive external opthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease set forth in any one or more of Tables 1-5, or any combination thereof.

Methods of Treating and/or Preventing Mitochondrial Diseases

Also described herein are methods of treating and/or preventing a mitochondrial disease or a symptom thereof in a subject in need thereof that can include diagnosing, prognosing, and/or monitoring a mitochondrial disease or a symptom thereof in the subject in need thereof as as previously described elsewhere herein, where the sample is from the subject in need thereof, and; administering one or more agent(s) or formulations thereof and or therapies to the subject in need thereof effective to treat and/or prevent the mitochondrial disease or symptom thereof.

In some embodiments, methods of diagnosing, prognosing, and/or monitoring a mitochondrial disease can include detecting mitochondrial DNA (mtDNA) heteroplasmy and cell type and/or cell state in a cell or cell population, wherein detecting can include detecting, in a sample comprising the cell or cell population, a cell signature in the cell or cell population, and detecting mtDNA heteroplasmy in the cell or cell population, where the cell signature and/or mtDNA heteroplasmy indicates at least cell type and/or cell state; and optionally repeating detecting mtDNA heteroplasmy and cell type and/or cell state one or more times over a period of time. In some embodiments, detecting mtDNA heteroplasmy can be repeated 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 times or more. In some embodiments, the period of time can range from 1 to 10 minutes, days, weeks, months, or years, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 minutes, days, weeks, months, or years.

In some embodiments, detecting mtDNA heteroplasmy and cell type and/or cell state one or more times over a period of time can allow for disease monitoring over that time, response to a treatment, and/or any other changes in a subject disease state, progress, and/or symptoms of the disease.

In some embodiments, the cell signature and/or mtDNA heteroplasmy detected by a method described herein can be compared to a where the cell signature and/or mtDNA heteroplasmy obtained from the same subject at a different time and/or a where the cell signature and/or mtDNA heteroplasmy obtained from a healthy or non-diseased subject.

In some embodiments, the cell signature comprises a chromatin accessibility signature, a gene expression signature, a protein expression signature, an epigenetic state signature, a cell surface marker expression signature, a cell activity signature, a phenotypic profile, a cell landscape, or a combination thereof. Signatures are discussed in greater detail elsewhere herein.

In some embodiments, detecting the signature and/or detecting mtDNA heteroplasmy is/are determined by a sequencing method. Suitable sequencing methods are described in greater detail elsewhere herein. In some embodiments, the sequencing method includes or is single cell RNA sequencing and/or mitochondrial DNA single cell ATAC-seq (mtscATAC-seq).

In some embodiments, detecting a cell signature comprises measuring a change in a distance in gene expression or accessible fragment space between two or more cell states. In some embodiments, the gene expression and/or accessible fragment space comprises 1 to 1000 or more accessible genes and/or accessible fragments, such as 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 or more genes and/or accessible fragments. In some embodiments, the gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments. In some embodiments, the distance in gene expression and/or accessible fragment space is measured by a Euclidean distance, Pearson coefficient, Spearman coefficient, or combination thereof.

In some embodiments, detecting mtDNA heteroplasmy comprises detecting one or more mutations in the mtDNA. In some embodiments, at least one of the one or more mutations are pathogenic. In some embodiments, the at least one of the one or more mtDNA mutations is selected from the group of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), one or more mutations as set forth in any one or more of Tables 1-5, or any combination thereof.

In some embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof. In some embodiments, the cell or cell population comprises one or more circulating mononuclear cell(s) and the cell signature comprises a circulating mononuclear cell signature. In some embodiments, the one or more circulating mononuclear cells comprise one or more peripheral blood mononuclear cells. In some embodiments, the one or more circulating mononuclear cells comprise lymphocyte(s), monocyte(s), dendritic cell(s) or a combination thereof. In some embodiments, the one or more circulating mononuclear cells comprise T cell(s), B cell(s), natural killer cell(s) or a combination thereof.

In some embodiments, the sample is a bodily fluid, a bodily excretion, a bodily secretion, a tissue, a cell or cells, or a combination thereof. In some embodiments, the sample is blood.

In some embodiments, the mitochondrial disease is a maternally inherited mitochondrial disease. In some embodiments, the mitochondrial disease is a heteroplasmic mitochondrial disease. In some embodiments, the mitochondrial disease is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease set forth in any one or more of Tables 1-5, or a combination thereof.

In some embodiments, the treatment can include administering a cell having a healthy or normal mitochondrial to a subject in need thereof. In some embodiments, the cell is an autologous cell that has had one or more of its mitochondria modified to change one or more pathologic mtDNA mutations from a pathologic to normal or non-pathologic sequence. The mtDNA can be modified ex vivo or in vivo. The mtDNA can be modified using any suitable polynucleotide modification method or technique. Suitable techniques include any polynucleotide guided nuclease system (e.g., any CRISPR-Cas System or IscB system).

Suitable polynucleotide modification techniques and systems (including guided nuclease systems) are known in the art. In general, In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molce1.2015.10.008. In some embodiments, the CRISPR-Cas system is capable of base editing or prime editing. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems. See e.g., Komor et al. 2016. Nature. 533:420-424; Nishida et al. 2016. Science. 353; and Li et al. Nat. Biotech. 36:324-327; Rees and Liu. 2018. Nat. Rev. Genet. 19(12): 770-788; Nishimasu et al. Cell. 156:935-949; Gaudeli et al. 2017. Nature. 551:464-471; International Patent Publication Nos. WO 2016/106236; WO 2018/213708, WO 2018/213726, WO 2019/005884, WO 2019/005886, and WO 2019/071048; and International Patent Applications No. PCT/US2018/067207, PCT/US2018/067225, PCT/US2018/05179 and PCT/US2018/067207 and PCT/US2018/067307, Anzalone et al. 2019. Nature. 576: 149-157, each of which is incorporated herein by reference.

In some embodiments, the polynucleotide modification system is a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

Generally, IscB systems include IscB proteins, which contain one or more domains capable of modifying a nucleic acid and can complex with hRNA. In some embodiments, the nucleic acid-guided nucleases herein may be IscB proteins. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide, and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g. spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

In some embodiments, the treatment or prevention is a mitochondrial replacement therapy. In some embodiments, the subject in need thereof can receive mitochondrial replacement therapy. Mitochondrial replacement therapy (MRT) refers to the replacement or the addition of mitochondria in one or more cells. In some embodiments, MRT can prevent or treat a disease or disorder. In some embodiments, MRT can partially or wholly restore normal function to a cell and/or tissue.

In some embodiments, the mitochondria administered to a subject in need thereof can be autologous. In some embodiments, the autologous mitochondria are unmodified prior to delivery. In some embodiments, the autologous mitochondria carry one or more modifications to mtDNA as compared to unmodified autologous mitochondria. In some embodiments, the modification(s) correct one or more pathologic mutations such that they are no longer associated with a pathologic condition. In some embodiments, the pathologic (or pathogenic) mutation(s) that can be corrected is/are any one or more of those listed in any one or more of Tables 1-5. In some embodiments, modification of mitochondria occurs ex vivo. The mtDNA can be modified in any suitable manner, including a polynucleotide guided nuclease (e.g., a CRISPR-Cas system or IscB system). In some embodiments, the cell having mitochondria to be modified is a somatic cell.

In some embodiments, the mitochondria administered to a subject in need thereof can be allogenic. In some embodiments, the allogenic mitochondria do not contain at least one pathologic mutation that is in the mitochondria of the subject in need thereof that the allogenic mitochondria are replacing.

In some embodiments, the replacement mitochondria can be delivered to a recipient cell or cells via any suitable method. Suitable delivery methods can include, but are not limited to, microinjection techniques. In some embodiments, the replacement mitochondria can be delivered to a somatic cell.

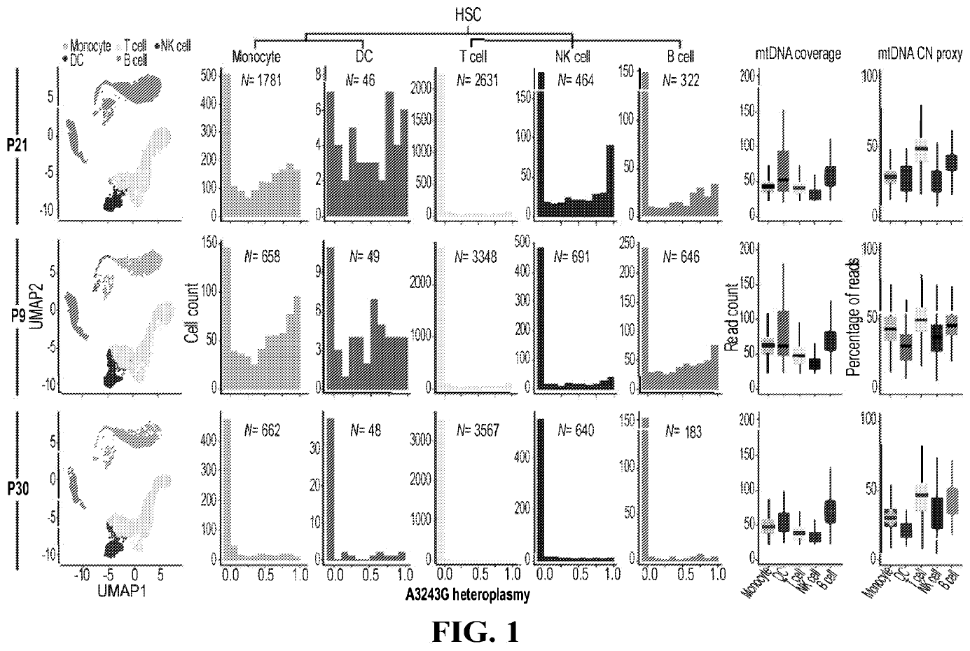

In some embodiments, a female can be homo or heteroplasmic for one or more mtDNA mutations that is/are pathologic. In some embodiments, it can be desirable not to pass the mutated mitochondria on to offspring. Thus, in some embodiments, an oocyte can be modified such that it contains nuclear material from the female having one or more pathologic mtDNA mutations and either modified autologous mitochondria that lack at least one of those pathologic mutations or healthy mitochondria that are native to the oocyte. In some embodiments, the one or more pathologic mutation(s) is/are any one or more from any one or more of Tables 1-5. As used in this context, “healthy” refers to unmodified mitochondria that lack at least one of those pathologic mutation such that the mitochondria of the recipient oocyte are normal in comparison to the mitochondria from female donating the nucleus or nuclear material. MRT for reproductive therapy is known. There are currently three primary procedures for accomplishing this daunting task; metaphase II spindle-chromosome complex (MII-SCC) transfer, pronuclear (PN) transfer, and germinal vesicular (GV) transfer (See e.g., FIG. 1 from Fogleman et al. 2016. Am J Stem Cells. 5(2): 39-52 and associated discussion). In MII-SCC transfer, the mature oocyte containing mutant mtDNA is progressed to metaphase II where the chromosomal material is arranged along the metaphase plate. Subsequently it can be harvested and implanted into a healthy, enucleated donor oocyte (See FIG. 1A from Fogleman et al. 2016. Am J Stem Cells. 5(2): 39-52 and associated discussion). This technique allows for the newly constructed oocyte to be fertilized by a viable sperm after the transfer occurs, but due to the nebulous nature of the spindle complex, carries the risk of extracting more cytoplasm and increasing the amount of mutated mtDNA that is concomitantly transferred (Tachibana et al. Nature. 2013; 493:627-631). PN transfer is the process by which the pronuclei, the nuclei of the sperm and oocyte before they fuse inside the oocyte, are removed from the parent zygote and are placed in a donor zygote that was previously fertilized and subsequently enucleated (Craven et al. Nature. 2010; 13:878-890) (See FIG. 1A from Fogleman et al. 2016. Am J Stem Cells. 5(2): 39-52 and associated discussion). This technique allows for the extraction of the two, well-defined pronuclei after the sperm has been introduced into the oocyte, potentially reducing the amount of cytoplasm that is transferred with the pronuclei and decreasing the carryover of mutated mtDNA (Craven et al. 2010).

In some embodiments, mitochondria having one or more pathologic mutations in the mtDNA in an oocyte can be modified using an appropriate mtDNA modification technique. In some embodiments, the mtDNA modification technique can be a polynucleotide guided nuclease system (e.g., a CRISPR-Cas system or an IscB system). In some embodiments, the oocyte can be modified ex vivo prior to an in vitro fertilization procedure. In some embodiments, the oocyte is from a non-human primate. In some embodiments, the oocyte is from a mammal. In some embodiments, the oocyte is from a human. In some embodiments, the oocyte is from a non-human animal.

In some embodiments, one or more mitochondria that have or are suspected of having pathologic mtDNA mutations can be removed from a cell prior to adding modified or unmodified replacement mitochondria to the cell.

### Screening for Modulating/Remodeling Agents

Also described herein are methods of screening for agents capable of modulating, modifying, and/or remodeling a mitochondria and/or mtDNA. Such agents can then be used treat and/or prevent a mtDNA disease or symptom thereof, such as any one or more of those described in greater detail elsewhere herein. Generally, screening for such agents can include exposing a subject, a cell, mitochondria and/or mtDNA (such as one having a mtDNA disease or a symptom thereof, and/or one or more mtDNA mutations described elsewhere herein) to a candidate or test agent and, after exposure, determining if modification, modulation, and/or remodeling of the cell, mitochondria, and/or mtDNA occurred in response to the exposure. A modulating (or modifying or remodeling) agent is identified as one that results in a change in mitochondria function and/or activity, a change in the mtDNA sequence, a change in cell function or activity related to mitochondrial activity or function, and/or a combination thereof. In some embodiments, the modulating (or modifying or remodeling) agent results in modification of a pathogenic mtDNA mutation such that it is non-pathogenic. In some embodiments, the modulating (or modifying or remodeling) agent results in modification in mtDNA heteroplasmy.

In some embodiments, the disclosed methods can be used to screen chemical libraries for agents that modulate chromatin architecture epigenetic profiles, and/or relationships thereof. By exposing cells, or fractions thereof, tissues, or even whole animals, to different members of the chemical libraries, and performing the methods described herein, different members of a chemical library can be screened for their effect on, e.g., mitochondria, mtDNA heteroplasmy, mtDNA disease, mtDNA and/or relationships thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Test agents can include any chemical or biological molecule or system or component thereof. In some embodiments, the test agent is a nucleic acid guided gene-editing system, such as a CRISPR-Cas or IscB system, or a component thereof (such as a guided nucleic acid modifying enzyme or guide polynucleotide).

In some embodiments, a method for identifying an agent capable of modulating, modifying and/or remodeling a mtDNA, mtDNA heteroplasmy, mitochondrial function, or a combination thereof of a cell or cell population as disclosed herein, comprising: a) applying a candidate agent to the cell or cell population, mitochondria, and/or mtDNA; b) detecting modulation of one or more phenotypic aspects of the mtDNA, mitochondria, cell and/or cell population by the candidate agent, thereby identifying the agent. The phenotypic aspects of the cell or cell population that is modulated can be a mitochondria and/or cell signature (e.g., a gene and/or protein expression signature) mitochondria and/or cell activity or function, and/or mtDNA heteroplasmy or sequence)).

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

### Kits

Any of the compounds, compositions, formulations, particles, cells, described herein or a combination thereof can be presented as a combination kit, such as a kit for determining segregation dynamics of mitochondrial DNA, detecting, diagnosing, prognosing, monitoring, treating and/or preventing a mtDNA disease, or a symptom thereof. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof can be in need of a treatment and/or prevention for a mitochondrial disease or a symptom thereof. In some embodiments, the mitochondrial disease is a disease as set forth in any one or more of Tables 1-5. In some embodiments, the instructions provide that the subject in need thereof to which the compounds, compositions, formulations, particles, cells, etc. or combinations thereof described herein or a combination thereof can be administered has one or more mtDNA mutations, such as any one or more of those set forth in any one or more of Tables 1-5.

Described herein are kits for use in diagnosing, prognosing, and/or monitoring a mitochondrial disease and/or determining segregation dynamics of mitochondrial DNA (mtDNA) that can include: a collection vessel configured to collect and/or contain a sample that can include a cell or cell population obtained from a body of a subject, where the sample is a bodily fluid, a bodily excretion, a bodily secretion, a tissue, a cell or cell population, or a combination thereof instructions fixed in a tangible medium of expression that provides direction to collect the sample in the collection vessel and determine
a) segregation dynamics of mtDNA,
b) a diagnosis of a mitochondrial disease,
c) a prognosis of a mitochondrial disease, or
d) a combination thereof,

and optionally monitor any one or more of (a)-(d) by a method that can include detecting mitochondrial DNA (mtDNA) heteroplasmy and cell type and/or cell state in the cell or cell population, where detecting can include detecting cell signature in the cell or cell population, and detecting mtDNA heteroplasmy in the cell or cell population, where the cell signature and/or mtDNA heteroplasmy indicates at least cell type and/or cell state; and optionally repeating detecting mtDNA heteroplasmy and cell type and/or cell state in the cell or cell population one or more times over a period of time.

In some embodiments, the cell signature comprises a chromatin accessibility signature, gene expression signature, protein expression signature, epigenetic state signature, a cell surface marker expression signature, a cell activity signature, a phenotypic profile, a cell landscape, or a combination thereof. In some embodiments, detecting the cell signature and/or detecting mtDNA heteroplasmy is/are determined by a single cell sequencing method. In some embodiments, the single cell sequencing method can include single cell RNA sequencing and/or mitochondrial DNA single cell ATAC-seq (mtscATAC-seq).

In some embodiments, detecting a cell signature comprises measuring a change in a distance in gene expression space and/or accessible fragment space between two or more cell states. In some embodiments, the gene expression and/or accessible fragment space comprises 1 to 1000 or more accessible genes and/or accessible fragments, such as 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 or more genes and/or accessible fragments.

In some embodiments, the gene expression and/or accessible fragment space comprises 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments. In some embodiments, the distance in gene expression and/or accessible fragment space is measured by a Euclidean distance, Pearson coefficient, Spearman coefficient, or combination thereof.

In some embodiments, detecting mtDNA heteroplasmy comprises detecting one or more mutations the mtDNA. In some embodiments, at least one of the one or more mutations are pathogenic. In some embodiments, the at least one of the one or more mtDNA mutations is selected from the group of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 1) repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and combinations thereof.

In some embodiments, the cell or cell population comprises one or more cells from a bodily fluid, bodily excretion, a bodily secretion, muscle, liver, kidney, lung, heart, brain, intestine, stomach, pancreas, bladder, skin, or a combination thereof. In some embodiments, the cell or cell population comprises one or more circulating mononuclear cell(s) and the cell signature is a circulating mononuclear cell signature. In some embodiments, the one or more circulating mononuclear cells comprise one or more peripheral blood mononuclear cells. In some embodiments, the one or more circulating mononuclear cells comprise lymphocyte(s), monocyte(s), dendritic cell(s) or a combination thereof. In some embodiments, the one or more circulating mononuclear cells comprise T cell(s), B cell(s), natural killer cell(s) or a combination thereof.

In some embodiments, the sample is a bodily fluid, a bodily excretion, a bodily secretion, a tissue, a cell or cells, or a combination thereof. In some embodiments, the sample is blood. In some embodiments, the mitochondrial disease is a maternally inherited mitochondrial disease. In some embodiments, the mitochondrial disease is a heteroplasmic mitochondrial disease.

In some embodiments, the mitochondrial disease is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease as set forth in any one or more of Tables 1-5, or a combination thereof.

In some embodiments, the collection vessel comprises a reagent effective to prepare and/or preserve the sample. In some embodiments, the collection vessel comprises a reagent effective to prepare and/or preserve the sample for detecting the cell signature and/or mtDNA heteroplasmy. In some embodiments, the collection vessel is physically and/or chemically configured to preserve and/or prepare the sample for detecting the circulating mononuclear cell signature and/or mtDNA heteroplasmy.

Further embodiments are illustrated in the following Examples which are given for illustrative purposes only and are not intended to limit the scope of the invention.

### EXAMPLES

### Example 1—Case Reports

Patient P21 is a 35-year-old man with MELAS, characterized by stroke-like episodes, failure to thrive, and steatohepatitis in whom clinical molecular testing identified the A3243G mutation without quantification of heteroplasmy. Patient P9 is a 29-year-old man with MELAS, characterized by sensorineural hearing loss (SNHL), migraine, epilepsy, ptosis, and stroke-like episodes. Based on clinical long-range polymerase chain reaction (PCR) and next-generation sequencing, this patient has A3243G heteroplasmy of 39% in whole blood. Patient P30 is a 60-year-old man with MELAS and associated SNHL, ptosis, stroke-like episodes, diabetes mellitus, skeletal myopathy with ragged red fibers, and cardiomyopathy with 77% A3243G heteroplasmy in skeletal muscle based on long-range PCR and next-generation sequencing.

### Example 2—Single Cell Analysis of Chromatin Accessibility and mtDNA in PBMCS

Using mtscATAC-seq, high quality sequencing libraries were generated to simultaneously evaluate cell type and heteroplasmy in thousands of individual cells per patient. From patient P21, we sequenced 6,687 cells (median of 7,045 nuclear fragments/cell); from patient P9, 6,003 cells (median of 6,672 nuclear fragments/cell); and from patient P30, 7,176 cells (median 8,146 nuclear fragments/cell) passing quality control (see Example 4).

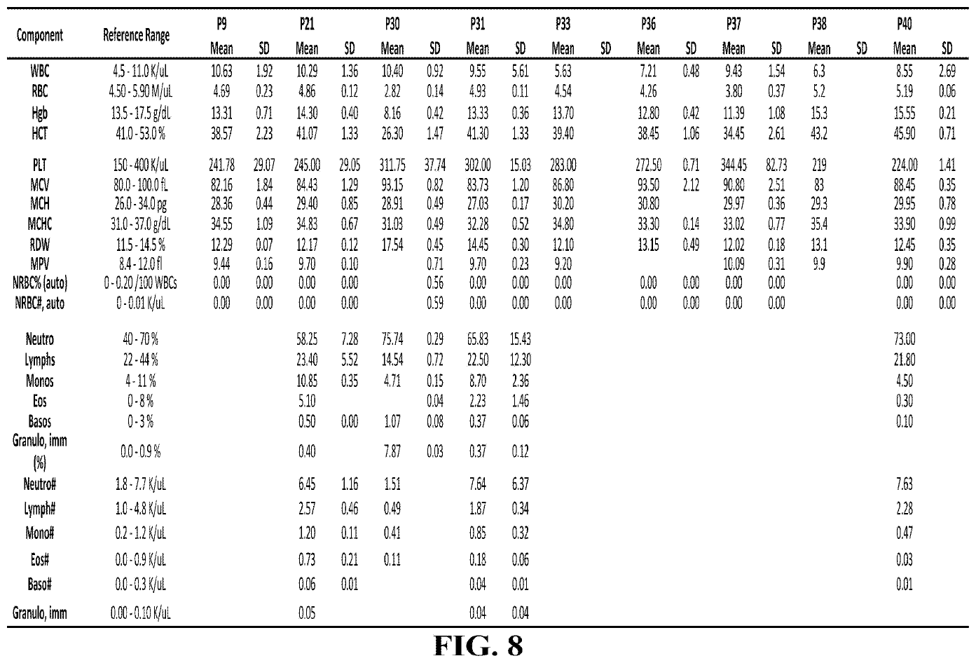

Using accessible chromatin signatures derived from nuclear genomic reads, cell states were defined using a latent semantic indexing (LSI) projection of each patient dataset onto a single-cell reference map of healthy donor PBMCs generated through a similar scATAC-seq protocol¹⁶. The clusters generated by each analysis were remarkably similar and had accessible chromatin profiles characteristic of canonical PBMC cell types (FIG. 1). The overall distributions of PBMC types identified by this protocol were similar for our patients compared to previously reported healthy donor PBMC datasets²¹. Furthermore, all patients showed normal representation of blood cell types on clinical CBCs (FIG. 8). Clinical heteroplasmy testing results for indicated tissue specimens are summarized in Table 6 (data shown where available).

Together, these results indicated no major perturbation in lineage frequencies in these patients.

TABLE 6
Clinical testing results and phenotypes of patients

| | | | | Oral | Skeletal | |
|---|---|---|---|---|---|---|
| ID | Age | Sex | Blood | Rinse | Muscle | Phenotype |
| P9 | 29 y | m | 39% | | | stroke, epilepsy, SNHL, urinary dysfunction, |
| | | | | | | cardiomyopathy, HA, ptosis, fatigue |
| P21 | 35 y | m | + | | | stroke, FTT, steatohepatitis |
| P30 | 60 y | m | | | 77% | stroke, cardiomyopathy, ptosis, bilateral SNHL, |
| | | | | | | DM, myopathy |
| P31 | 47 y | f | | 25% | | SNHL, HA, possible GI dysmotility, autonomic |
| | | | | | | dysfunction, fatigue |
| P33 | 65 y | f | | 22.5% | | mild myopathy, ptosis, GI dysmotility, |
| | | | | | | deafness, DM, fatigue, exercise intolerance, HA |
| P36 | 53 y | f | 20% | | | GI dysmotility, HA, burning mouth syndrome, |
| | | | | | | SNHL, fatigue, autonomic dysfunction, |
| | | | | | | myopathy, ptosis |
| P37 | 19 y | f | 46% | | | seizures, lactate peak on MRS, cardiomyopathy |
| P38 | 33 y | m | + | | | DM, hearing loss |
| P40 | 35 y | m | + | | | myoclonus, hearing loss |

The notation “+” denotes presence of the A3243G mutation by restriction-enzyme based molecular blood testing, without heteroplasmy quantification. Patient clinical phenotypes are summarized. Abbreviations include: m = male, f = female, SNHL = sensorineural hearing loss, HA = headache, FTT = failure to thrive, DM = diabetes mellitus, GI = gastrointestinal, MRS = magnetic resonance spectroscopy.

### Example 3—Cell Type Specific Heteroplasmy Determination

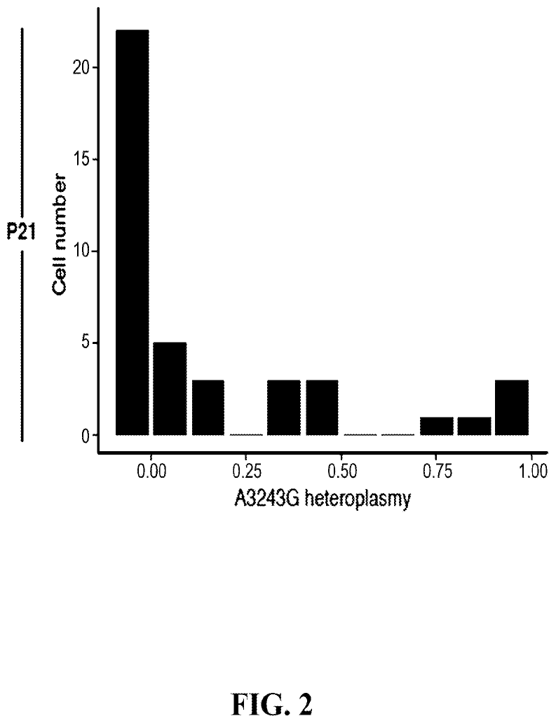

Heteroplasmy was examined across PBMC cell types, restricting the analyses to those cells with at least 20× coverage at position m.A3243. All cell types exhibited a broad spectrum of heteroplasmy, ranging from no A3243G alleles detected to exclusively A3243G mutations detected within each lineage, even in patients with low (<10%) bulk heteroplasmy (FIG. 1). This observation holds true even upon restricting to 100λ coverage at m.3243 in patient P21, where we still observe cells with exclusively wildtype or with exclusively mutant alleles (FIG. 2).

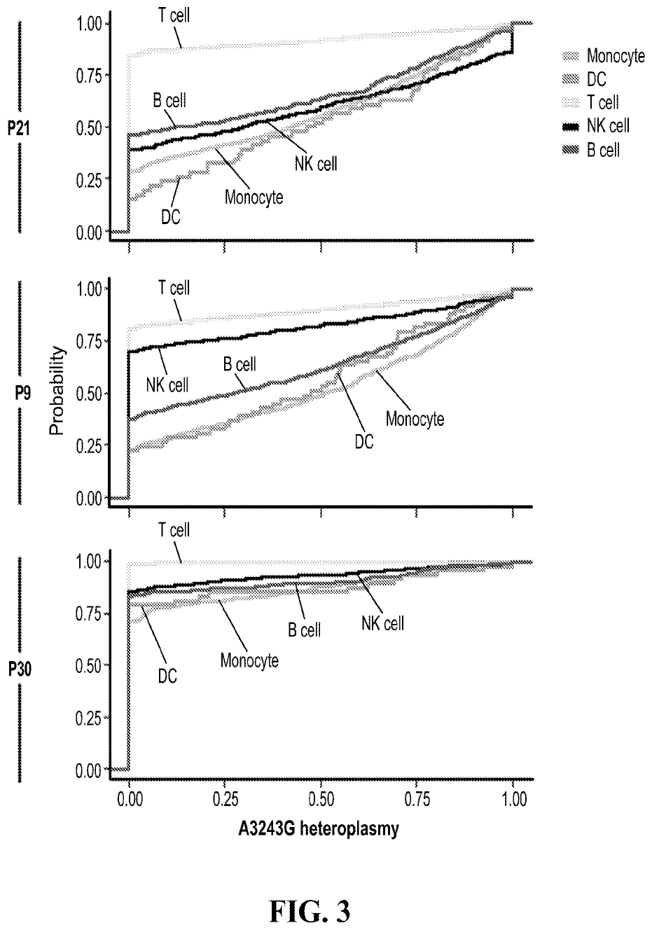

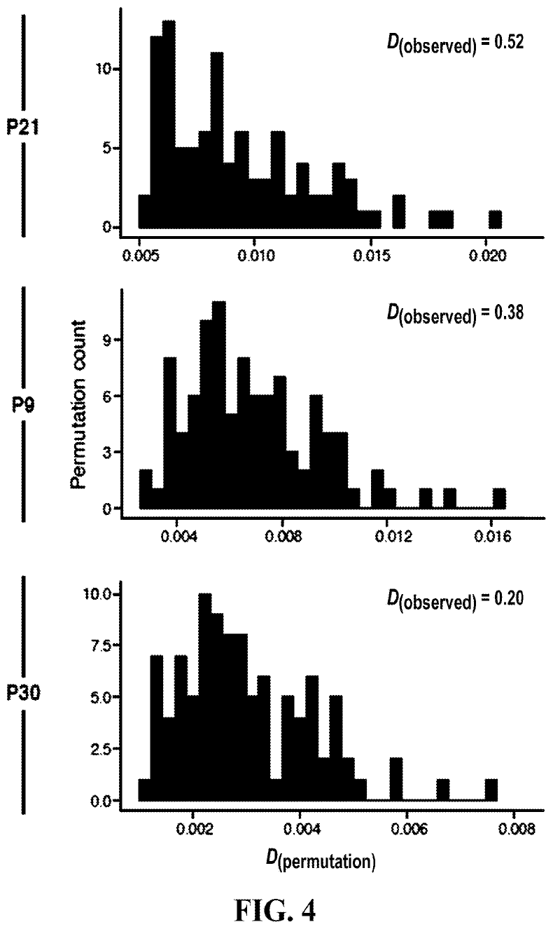

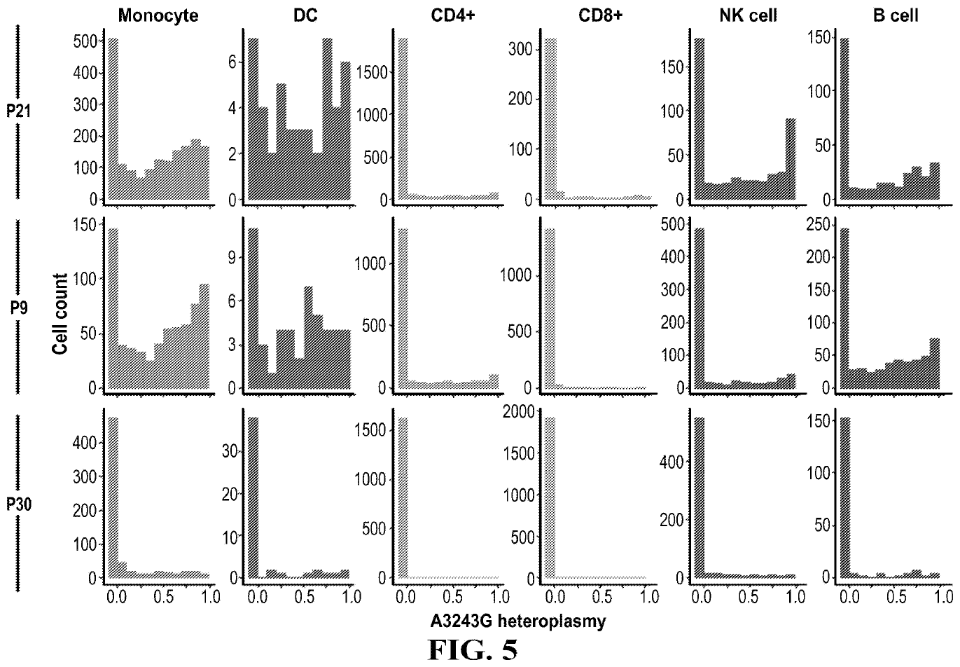

However, in T cell lineages, heteroplasmy values were significantly lower than in cells of other lineages (FIG. 1). The distribution of heteroplasmy for the T cells versus all lineages was compared (FIG. 3) and a statistically significantly left shifted distribution was observed based on a two sample Kolmogorov-Smirnov (K-S) D-statistic. The D-statistic comparing T cells to total PBMCs was 0.52 (D_{α}=0.03 for α=0.05), 0.38 (D_{α}=0.03 for α=0.05), 0.20 (D_{α}=0.03 for α=0.05) for P21, P9, P30, respectively. The large, non-zero D statistic values observed indicate that the distributions of A3243G heteroplasmy in T cells is not identical to the distribution of heteroplasmy in PBMCs. In all three subjects, the observed D was significant based on empirical permutation testing (P<0.01, FIG. 4). In cumulative distribution frequency plots of A3243G heteroplasmy by cell type, the T cell A3243G heteroplasmy frequency distribution is consistently the most left-shifted. This pattern holds when cells were further subdivided into specific subsets, with CD4+ and CD8⁺ T cell clusters each demonstrating lower median heteroplasmy compared to other populations (FIG. 5).

The surprising result of reduced heteroplasmy in the T cell lineage was validated and extended with traditional bulk heteroplasmy analysis (Table 7) of these and additional patients. In these validation studies, T cells were purified using either of two methods (FACS or bead-based negative selection) and assessed heteroplasmy by PCR amplification of the m.3243 region and next generation sequencing. First, using these orthogonal methods, the findings of reduced T cell heteroplasmy in two of the tested subjects for whom additional blood was available (P9, P30) were validated. These methods were then used to compare heteroplasmy in T cells versus total PBMCs in six additional patients who had heteroplasmic A3243G disease, but have not experienced stroke-like episodes (clinical testing and presentations summarized in Table 6). In all six additional cases, T cell populations demonstrated lower heteroplasmy (Table 7). Table 7 shows a validation of reduced A3243G heteroplasmy in T cells by bulk sequencing. Hence, these observations of reduced heteroplasmy appear to be robust across multiple methodologies.

TABLE 7
Bulk Heteroplasmy Measurements

| | Age | Sex | Total | T cell- | |
|---|---|---|---|---|---|
| Subject ID | (years) | (M/F) | PBMCs | depleted | T cells |
| P9 | 29 | M | 28.8% | PBMCs | 9.9% |
| P30 | 60 | M | 10% | 9% | 1% |
| P31 | 47 | F | | 5% | 1% |
| P33 | 65 | F | 6% | 6% | 1% |
| P36 | 53 | F | 16.3% | | 5.9% |
| P37 | 19 | F | 42.1% | | 24.8% |
| P38 | 33 | M | 46.1% | | 32% |
| P40 | 35 | M | 7.9% | | 3.2% |

Percent A3243G heteroplasmy was measured for total PBMCs, flow sorted T cell-depleted PBMCs, and T cells purified by negative selection as measured by next generation sequencing of a PCR amplicon encompassing the m.3243 position. Due to insufficient sample availability, bulk sequencing was not performed for patient

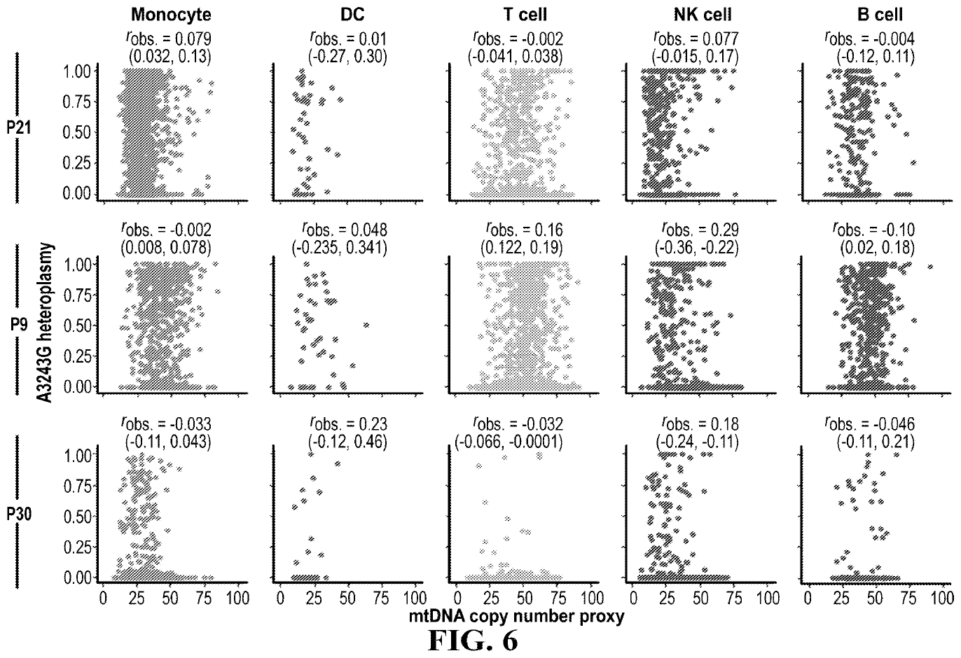

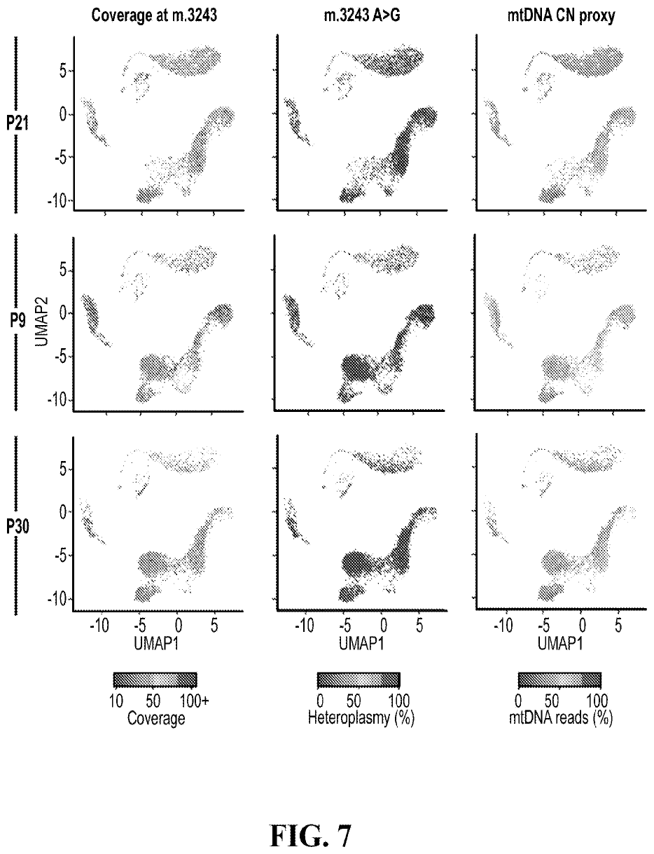

Next it was examined if differences in mtDNA copy number might account for the observed T cell-specific depletion of the heteroplasmic mutation. T cell activation induces mitochondrial biogenesis^{22,23}, and in worms, regulation of mtDNA copy number is associated with mtDNA surveillance²⁴. While a proxy for mtDNA copy number varied by cell type (FIG. 1), it did not show a relationship to heteroplasmy within any cell type (FIGS. 6-7).

Heteroplasmic dynamics is one of the most clinically challenging and scientifically fascinating aspects of mtDNA disease. Bulk heteroplasmy measurements across tissue types and kindreds have failed to explain the origin, transmission, variability, and pathogenic mechanisms of pathologic mtDNA heteroplasmy. Blood heteroplasmy, however, has long shown several peculiarities, including lower bulk heteroplasmy compared to other tissues^{1,25,7,8,9}, a weaker direct association with disease severity compared to urine sediment (another clinically tested biospecimen)^{1,7,25}, and a tendency to decline with age (e.g., ^{7,8,26,27,28}). At present, the mechanisms governing these complex dynamics are not known, but prior studies predict the existence of genetic factors that influence tissue-specific heteroplasmy^{1,2,29}.

Single cell analysis of heteroplasmy holds promise to elucidate mechanisms regulating mtDNA heteroplasmic dynamics, but patient studies to date have largely been restricted to the study of one cell type at a time (typically oocytes) at limited scale. Previous reports examined heteroplasmy in 82 oocytes¹⁴ and 8 pancreatic beta cells³⁰ in a single A3243G patient each. Similarly, studies of T8993G heteroplasmy have reported restriction enzyme based analysis in cells from single donors, including 87 oocytes¹¹, 2 blastomeres¹², and 30 lymphocytes¹³.

Emerging single cell technologies facilitated the study of heteroplasmy at massive scale and high-throughput¹⁵ and allowed the demonstration of A3243G heteroplasmy in thousands of individual cells representing multiple lineages arising from a common blood stem/progenitor pool in three unrelated patients as presented herein.

By investigating single cell heteroplasmy on this scale, the Examples herein demonstrate an unexpected observation about A3243G heteroplasmy across somatic lineages. In each patient and cell type studied, irrespective of median heteroplasmy in bulk, it was possible to identify individual cells spanning a broad range of heteroplasmy, from those devoid of detectable mutant allele to cells in which we only detected mutant alleles. This distribution, however, is dramatically left-shifted and tends to be significantly lower in T cell lineages. In the Examples herein, in all 3 of 3 patients investigated by mtscATAC-seq (FIG. 1), as well as all 6 of 6 additional patients investigated by bulk heteroplasmy analysis (Table 7), reduced heteroplasmy in T cells relative to all PBMCs was observed. This observation is not consistent with purely random segregation of the A3243G mutation.

Without being bound by theory, these observations may reflect the action of purifying selection against the pathogenic mtDNA allele in the T cell lineage. Given that the common lymphoid progenitor is the final branch point between T cell, B cell and NK cell lineages, selection against higher heteroplasmy T cells would be expected to be distal to this developmental stage. The A3243G mutation is known to cause a deficiency in the activity of complex I of the electron transport chain^{31,32}, and multiple previous studies in mouse models have shown that complete knockouts of nuclear encoded mitochondrial proteins in the whole organism^{33,34}, at specific developmental phases³⁵, or selectively in T cells³⁶ can impair T cell development, homeostasis, and/or immune function. Thus, a cell-intrinsic or T cell-specific process in the bone marrow, the thymus, or in the periphery may select against high heteroplasmy, with features unique to T cell biology being important candidates. Developmentally, A3243G-related mitochondrial dysfunction might, for example, present an insurmountable barrier in positive thymic selection or serve as a trigger for elimination during negative selection. Alternatively, immune mechanisms may be in place that actively surveil protein products of mutant mtDNA molecules and eliminate such cells in the T cell lineage. For example, mutations in the MT-ND1 gene have been shown to produce a peptide that is recognized by cytotoxic T cells in mice³⁷. This may also represent a compensatory mechanism to ensure that T cells with dysfunctional mitochondria do not activate inflammatory responses³⁸.

Understanding heteroplasmy dynamics within blood lineages has important clinical implications. First, these data can suggest that the lower heteroplasmy detected in blood may arise specifically from T cells and has implications for understanding the role of the immune system in the pathogenesis of mitochondrial disease, whose triggers often include infections. Second, this work can have implications for the diagnosis and monitoring of patients with heteroplasmic disease. Presently, clinical sequencing of blood to diagnose mtDNA disorders is controversial in part because of the longstanding observation of reduced heteroplasmy in the blood²⁶. Aspects of these Examples can at least demonstrate an approach to improve clinical detection of the heteroplasmic A3243G allele, namely, clinical sequencing of defined and purified lineage.

### Example 4—Methods for Examples 1-3

### Single Cell Accessible Chromatin and Mitochondrial Genotyping

Patient venous blood was collected at clinical baseline and purified peripheral blood mononuclear cells (PBMCs). Cells were stained for viability and applied anti-h (human) CD45 antibodies prior to fixation and performed Fluorescence-Activated Cell Sorting (FACS) to exclude dead and non-leukocyte cells (CD45^{neg}). MtscATAC-seq libraries were generated using a 10× Chromium Controller and a modified Chromium Single Cell ATAC Library & Gel Bead Kit protocol, followed by paired-end sequencing using an Illumina NextSeq 500 platform (2× 72 base pair reads).

Additional Details. Venous blood was collected from additional patients at clinical baseline using sodium heparin CPT tubes (BD Biosciences #362753) and peripheral blood mononuclear cells (PBMCs) were purified per manufacturer instructions. PBMCs were cryopreserved prior to use. Upon thawing, cells were stained with a fixable viability (Zombie Green, Biolegend #423111) and APC-conjugated anti-hCD45 (Biolegend #304012) stains. After washing, PBMCs were fixed in 1% formaldehyde (FA; ThermoFisher #28906) in PBS for 10 min at RT, quenched with glycine solution to a final concentration of 0.125M before washing cells once with PBS supplemented with 0.4% bovine serum albumin, and subsequent in PBS alone via centrifugation at 400 g, 5 min, 4 degrees C. Fluorescence-Activated Cell Sorting (FACS) was then performed to exclude dead and non-leukocyte cells.

MtscATAC-seq libraries were generated using the 10× Chromium Controller and the Chromium Single Cell ATAC Library & Gel Bead Kit (#1000111) according to the manufacturer's instructions (CG000169-Rev C; CG000168-Rev B) but with the following modifications: 1.5 ml-2 ml DNA LoBind tubes (Eppendorf) were used to wash PBMCs in PBS and downstream processing steps. Cells were subsequently treated with lysis buffer (10 mM Tris-HCL pH 7.4, 10 mM NaCl, 3 mM MgCl₂, 0.1% NP40, 1% BSA) for 3 min on ice, followed by adding 1 ml of chilled wash buffer and inversion (10 mM Tris-HCL pH 7.4, 10 mM NaCl, 3 mM MgCl₂, 1% BSA) before centrifugation at 500 g, 5 min, 4 degrees C. The supernatant was discarded, and cells were diluted in 1× Diluted Nuclei buffer (10× Genomics) before counting using Trypan Blue and a Countess II FL Automated Cell Counter. If large cell clumps were observed a 40 μm Flowmi cell strainer was used prior to processing cells according to the Chromium Single Cell ATAC Solution user guide with no additional modifications. Briefly, after tagmentation, the cells were loaded on a Chromium controller Single-Cell Instrument to generate single-cell Gel Bead-In-Emulsions (GEMs) followed by linear polymerase chain reaction (PCR) as described in the 10× User Guide. After breaking the GEMs, the barcoded tagmented DNA was purified and further amplified to enable sample indexing and enrichment of scATAC-seq libraries. The final libraries were quantified using a Qubit dsDNA HS Assay kit (Invitrogen) and a High Sensitivity DNA chip run on a Bioanalyzer 2100 system (Agilent). Paired-end sequencing performed using an Illumina NextSeq platform using 150 base pair reads.

### Data Analysis.

Raw sequencing reads were demultiplexed and aligned to the hg19 reference genome using the CellRanger-ATAC v1.0 software. Cells were identified as barcodes that met the following criteria: (1) ≥1,000 unique fragments mapping to the nuclear genome; (2) ≥40% of nuclear fragments overlapping a previously-established chromatin accessibility peak set in the hematopoietic system¹⁶; and (3) mean mtDNA coverage of ≥20× at position 3243 in the mtDNA genome. From the output of the CellRanger-ATAC call, we quantified mtDNA using the mgatk package¹⁵.

Cell types were computationally identified based on chromatin accessibility. Briefly, cells were reprocessed from a healthy individual¹⁷ to define axes of variation using Latent Sematic Indexing (LSI) and Uniform Manifold Approximation and Projection (UMAP). Next, projected patient-derived cells were projected onto this reduced-dimension space using the LSI/UMAP loadings as previously described¹⁸. k-nearest neighbors (k=20) was used to generate twelve data-driven clusters via Louvain community detection, which were mapped onto five major expected cell types in PBMCs (monocytes, dendritic cells (DCs), T cells, B cells, and natural killer (NK) cells). The clustering was robust to the choice of k_(see Additional Details below). All cell types were classified in patient samples by LSI projection and minimum distance to cluster medoids. For visualization, two dimensional representations of patient PBMC data were produced by projecting the 25 LSI dimensions onto the pre-trained UMAP model as previously reported¹⁸.

All cells used in these analyses were filtered to exclude cells with <20× coverage at position m.3243. Outliers with m.3243 coverage of >1.5 interquartile ranges above the third quartile were also excluded to avoid inclusion of artefactual sequencing multiplets. The fraction of total read fragments aligning to the mitochondrial genome were calculated in each cell as a proxy for mtDNA copy number (CN).

To compare the distribution of heteroplasmy in T cells versus all PBMCs, we employed a Kolmogorov-Smirnov two-sample test statistic, D, which defined as the maximum difference between cumulative distributions at any given point and is expected to approach zero for identical distributions and as high as 1 when very shifted. To evaluate the significance of the observed test statistic, empirical permutation testing was used. Briefly, for a given patient, the cell type label (i.e., T cell or not T cell, preserving the proportion of T cells observed in that patient) was permutated. Then the two-sample K-S test statistic was computed using the permuted data, and this procedure was repeated 100 times. As a measure of statistical significance, the fraction of K-S statistics calculated on permuted data that exceeded the observed K-S test statistic for the real data was counted. The R base and stats package version 3.5.1 and base version 3.5.1 was used to perform these computations. Data analyses and visualization were also conducted using R.

Additional Details. Raw sequencing reads were demultiplexed and aligned to the hg19 reference genome using the CellRanger-ATAC v1.0 software. Cells were identified as barcodes that met the following criteria: (1) presence of at least 1,000 unique fragments mapping to the nuclear genome; (2) at least 40% of nuclear fragments overlapping a previously-established chromatin accessibility peak set in the hematopoietic system¹⁶, and (3) had a mean mtDNA coverage of at least 20× at position 3243 in the mtDNA genome. From the output of the CellRanger-ATAC call, we quantified heteroplasmy at all loci, including A3243G, in the mitochondrial genome using the mgatk package, which is available at https://github.com/caleblareau/mgatk. Outliers with m.3243 coverage of >1.5 interquartile ranges above the third quartile were also excluded to avoid artefactual sequencing multiplets.

A computational strategy was applied to identify cell types independent of possible alterations in chromatin accessibility caused by the pathogenic allele. This was achieved by first defining axes of variation in a healthy individual and then projecting new (patient) cells onto this existing space, utilizing Latent Sematic Indexing (LSI) and Uniform Manifold Approximation and Projection (UMAP) as previously described¹⁸. Specifically, a binarized matrix of chromatin accessibility peaks was generated for about 10,000 PBMCs derived from a healthy donor¹⁷ were reduced into 25 dimensions via LSI and those were subsequently reduced to 2 dimensions via UMAP for visualization. Using the 25 dimensions in LSI space a k nearest neighbors graph (k=20) was constructed, and twelve data-driven clusters were obtained by a Louvian community clustering on this graph, which were annotated by five major cell types expected in PBMCs.

The selection of k=20 was chosen as it serves as a default value consistently used in common single-cell analyses tools, including the statistical frameworks used herein^{18,41}. To verify that the results are not sensitive to this choice of parameter, the Adjusted Rand Index (ARI) for values of k=10, 15, 20, 25, and 30 was computed to compare the clustering results under variable choice of this parameter. An ARI value of 0 is indicative of no concordance between clusters (random) whereas a value of 1 represents perfect concordance. When analyzing these in the context of our data, we found that for all values of k, the ARI to the definitions used in the manuscript exceed 0.9, reflective of very robust results irrespective of the choice of parameter for this value.

Next, all patient cell types were classified by projecting chromatin accessibility data onto this 25-dimensional space and assigning cell types based on minimum distance to cluster medoids. Finally, two dimensional representations of patient data were produced by projecting the 25 LSI dimensions onto the pre-trained UMAP model as previously reported¹⁸. In the assignment of cells to their closest reference cluster, the minimum Euclidean distance between the reference medoid and the individual cell in the reduced dimension space defined by the LSI components was used. While a minimum distance for the classification was not required, a mean 2-fold distance between the individual cells and closest reference cluster medoid (0.011) compared to the second closest cluster medoid (0.025) was observed. These results support that the classification was robust in this high-dimensional space.

To test for correlations between A3243G heteroplasmy and the proxy of mtDNA copy number (the ratio of reads aligning to the mitochondrial and nuclear genomes), Spearman rank correlation coefficients were calculated for each dataset in R using cor.test (Package stats version 3.5.1 Index). 95% confidence intervals were estimated from the distributions of the test statistic from 10,000 datasets generated from the observed dataset by bootstrapping with replacement. These computations were performed using the boot function (Package boot version 1.3-23) and the boot.ci function, basic 95% confidence intervals (Package boot version 1.3-23). We calculated critical values (rₛ) for Spearman rank correlation coefficients for α=0.05 as follows: rₛ=+z/(\sqrt{n−1}).

### Bulk Sequencing and Heteroplasmy Analysis

PBMCs were stained with antibodies against hCD45 and hCD56 and used FACS to purify T cell and T cell-depleted PBMC populations from which DNA was extracted. Small amplicons centered on m.3243 were generated by polymerase chain reaction (PCR) and sequenced on an Illumina MiSeq platform. Reads were aligned using BWA¹⁹ and analyzed them with Samtools²⁰. T cells were additionally purified using magnetic bead negative selection kits. DNA from purified T cells and total PBMCs was extracted and forwarded to generation of m.3243 region PCR amplicons for Sanger sequencing.

Additional Details. Cryopreserved PMBCs were stained with anti-human CD45-APC (Biolegend #304012), OKT3 anti human CD3e-FITC Ab (Biolegend #317305), and Pacific Blue™ anti-human CD56 clone HCD56 (Biolegend #318325). FACS was then used to purify T cell and T cell-depleted PBMC populations from which DNA was extracted (Qiagen #69504). Small amplicons containing the m.3243 locus and surrounding region were generated by (PCR) and used to generate libraries for sequencing on an Illumina MiSeq platform. Heteroplasmy was called from this data using Samtools²⁰. The m.3243 region was amplified by PCR and Sanger sequencing performed by conventional methods (Genewiz). Primer sequences were 5′-CGCCTTCCCCCGTAAATGA-3′ (SEQ ID NO: 8) (forward), 5′-GGGGCCTTTGCGTAGTTGT-3′ (SEQ ID NO: 9) (reverse) for amplicon amplification and next generation sequencing.

### REFERENCES FOR EXAMPLES

1. Pickett S J, Grady J P, Ng Y S, et al. Phenotypic heterogeneity in m.3243A>G mitochondrial disease: The role of nuclear factors. Ann Clin Transl Neurol 2018;
2. Jenuth J P, Peterson A C, Shoubridge E A. Tissue-specific selection for different mtDNA genotypes in heteroplasmic mice. Nat Genet 1997;
3. Manwaring N, Jones M M, Wang J J, et al. Population prevalence of the MELAS A3243G mutation. Mitochondrion 2007;
4. Elliott H R, Samuels D C, Eden J A, Relton C L, Chinnery P F. Pathogenic Mitochondrial DNA Mutations Are Common in the General Population. Am J Hum Genet 2008;
5. Goto Y I, Nonaka I, Horai S. A mutation in the tRNALeu(UUR) gene associated with the MELAS subgroup of mitochondrial encephalomyopathies. Nature 1990;
6. Hirano M, Ricci E, Richard Koenigsberger M, et al. MELAS: An original case and clinical criteria for diagnosis. Neuromuscul Disord 1992;
7. Grady J P, Pickett S J, Ng Y S, et al. mtDNA heteroplasmy level and copy number indicate disease burden in m.3243A>G mitochondrial disease. EMBO Mol Med 2018;
8. De Laat P, Koene S, Van Den Heuvel L P W J, Rodenburg R J T, Janssen M C H, Smeitink J A M. Clinical features and heteroplasmy in blood, urine and saliva in 34 Dutch families carrying the m.3243A>G mutation. J Inherit Metab Dis 2012;
9. Maeda K, Kawai H, Sanada M, et al. Clinical phenotype and segregation of mitochondrial 3243A>G mutation in 2 pairs of monozygotic twins. JAMA Neurol 2016;
10. Hyslop L A, Blakeley P, Craven L, et al. Towards clinical application of pronuclear transfer to prevent mitochondrial DNA disease. Nature 2016;
11. Blok R B, Gook D A, Thorburn D R, Dahl H H M. Skewed segregation of the mtDNA nt 8993 (T→G) mutation in human oocytes. Am J Hum Genet 1997;
12. Steffann J, Frydman N, Gigarel N, et al. Analysis of mtDNA variant segregation during early human embryonic development: A tool for successful NARP preimplantation diagnosis. J Med Genet 2006;
13. Gigarel N, Ray P F, Burlet P, et al. Single cell quantification of the 8993T>G NARP mitochondrial DNA mutation by fluorescent PCR. Mol Genet Metab 2005;
14. Brown D T, Samuels D C, Michael E M, Turnbull D M, Chinnery P F. Random genetic drift determines the level of mutant mtDNA in human primary oocytes. Am J Hum Genet 2001;
15. Caleb A. Lareau, Leif S. Ludwig, Christoph Muus, Satyen H. Gohil, Tongtong Zhao, Zachary Chiang, Karin Pelka, Jeffrey M. Verboon, Wendy Luo, Elena Christian, Daniel Rosebrock, Gad Getz, Genevieve M. Boland, Fei Chen, Jason D. Buenrostro, Nir Hacohen, Cath V G S. Massively parallel joint single-cell mitochondrial DNA genotyping and chromatin profiling reveals properties of human clonal variation. Nat Biotechnol 2020;
16. Ulirsch J C, Lareau C A, Bao E L, et al. Interrogation of human hematopoiesis at single-cell and single-variant resolution. Nat Genet 2019;
17. Satpathy A T, Granja J M, Yost K E, et al. Massively parallel single-cell chromatin landscapes of human immune cell development and intratumoral T cell exhaustion. Nat Biotechnol 2019;
18. Granja J M, Klemm S, McGinnis L M, et al. Single-cell multiomic analysis identifies regulatory programs in mixed-phenotype acute leukemia. Nat. Biotechnol. 2019;
19. H L, R D. Fast and accurate short read alignment with Burrows-Wheeler Transform. Bioinformatics 2009;
20. Li H, Handsaker B, Wysoker A, et al. The Sequence Alignment/Map format and SAMtools. Bioinformatics 2009;
21. Ludwig L S, Lareau C A, Bao E L, et al. Transcriptional States and Chromatin Accessibility Underlying Human Erythropoiesis. Cell Rep 2019;
22. Ron-Harel N, Santos D, Ghergurovich J M, et al. Mitochondrial Biogenesis and Proteome Remodeling Promote One-Carbon Metabolism for T Cell Activation. Cell Metab 2016;
23. Filograna R, Koolmeister C, Upadhyay M, et al. Modulation of mtDNA copy number ameliorates the pathological consequences of a heteroplasmic mtDNA mutation in the mouse. Sci Adv 2019;
24. Haroon S, Li A, Weinert J L, et al. Multiple Molecular Mechanisms Rescue mtDNA Disease in C. elegans. Cell Rep 2018;
25. Fayssoil A, Laforet P, Bougouin W, et al. Prediction of long-term prognosis by heteroplasmy levels of the m.3243A>G mutation in patients with the mitochondrial encephalomyopathy, lactic acidosis and stroke-like episodes syndrome. Eur J Neurol 2017;
26. Rahman S, Poulton J, Marchington D, Suomalainen A. Decrease of 3243 A→G mtDNA Mutation from Blood in MELAS Syndrome: A Longitudinal Study. Am J Hum Genet 2002;
27. Pyle A, Taylor R W, Durham S E, et al. Depletion of mitochondrial DNA in leucocytes harbouring the 3243A→G mtDNA mutation. J Med Genet 2007;
28. Mehrazin M, Shanske S, Kaufmann P, et al. Longitudinal changes of mtDNA A3243G mutation load and level of functioning in MELAS. Am J Med Genet Part A 2009;
29. Jokinen R, Marttinen P, Sandell H K, et al. Gimap3 regulates tissue-specific mitochondrial DNA segregation. PLoS Genet 2010;
30. Lynn S, Borthwick G M, Charnley R M, Walker M, Turnbull D M. Heteroplasmic ratio of the A3243G mitochondrial DNA mutation in single pancreatic beta cells. Diabetologia 2003;
31. Shinozawa K, Nishizawa M, Tanaka K, Atsumi T, Ohama E. A mitochondrial encephalomyopathy: a case of a defect of complex I in the electron transport chain. Clin Neurol 1987;
32. Tanaka M, Nishikimi M, Suzuki H, et al. Deficiency of subunits of complex I or I V in mitochondrial myopathies: Immunochemical and immunohistochemical study. J Inherit Metab Dis 1987;
33. Cabon L, Bertaux A, Brunelle-Navas M N, et al. AIF loss deregulates hematopoiesis and reveals different adaptive metabolic responses in bone marrow cells and thymocytes. Cell Death Differ 2018;
34. Ramstead A G, Wallace J A, Lee S H, et al. Mitochondrial Pyruvate Carrier 1 Promotes Peripheral T Cell Homeostasis through Metabolic Regulation of Thymic Development. Cell Rep 2020;
35. Simula L, Pacella I, Colamatteo A, et al. Drp1 Controls Effective T Cell Immune-Surveillance by Regulating T Cell Migration, Proliferation, and cMyc-Dependent Metabolic Reprogramming. Cell Rep 2018;
36. Tarasenko T N, Pacheco S E, Koenig M K, et al. Cytochrome c Oxidase Activity Is a Metabolic Checkpoint that Regulates Cell Fate Decisions During T Cell Activation and Differentiation. Cell Metab 2017;
37. Loveland B, Wang C R, Yonekawa H, Hermel E, Lindahl K F. Maternally transmitted histocompatibility antigen of mice: A hydrophobic peptide of a mitochondrially encoded protein. Cell 1990;
38. Desdin-Mico G, Soto-Heredero G, Aranda J F, et al. T cells with dysfunctional mitochondria induce multimorbidity and premature senescence. Science 2020;
39. Parikh S, Goldstein A, Koenig M K, et al. Diagnosis and management of mitochondrial disease: A consensus statement from the Mitochondrial Medicine Society. Genet. Med. 2015;
40. Regev A, Teichmann S, Lander E, et al. Science Forum: The Human Cell Atlas. Elife 2017;
41. Stuart T, Butler A, Hoffman P, et al. Comprehensive Integration of Single-Cell Data. Cell 2019.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of determining segregation dynamics of mitochondrial DNA (mtDNA) comprising:
detecting mtDNA heteroplasmy and cell type, cell state, or both in a cell or cell population, wherein detecting comprises,
detecting, in a sample comprising the cell or cell population, a cell signature in the cell or cell population, and
detecting mtDNA heteroplasmy in the cell or cell population,
wherein the cell signature and/or mtDNA heteroplasmy indicates at least cell type, cell state, or both.

2. The method of claim 1, wherein the cell signature comprises a chromatin accessibility signature, a gene expression signature, a protein expression signature, an epigenetic state signature, a cell surface marker expression signature, a cell activity signature, a phenotypic profile, a cell landscape, or a combination thereof.

3. The method of claim 1, wherein detecting the cell signature and/or detecting mtDNA heteroplasmy is/are determined by a sequencing method.

4. The method of claim 3, wherein the sequencing method comprises single cell RNA sequencing and/or mitochondrial DNA single cell ATAC-seq (mtscATAC-seq).

5. The method of claim 1, wherein detecting a cell signature comprises measuring a change in a distance in gene expression space between two or more cell states and/or measuring a change in a distance in accessible fragment space between two or more cell states.

6. The method of claim 5, wherein the gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

7. The method of claim 5, where the distance in gene expression and/or accessible fragment space is measured by a Euclidean distance, Pearson coefficient, Spearman coefficient, or combination thereof.

8. The method of claim 1, wherein detecting mtDNA heteroplasmy comprises detecting one or more mutations of the mtDNA.

9. The method of claim 8, wherein at least one of the one or more mutations are pathogenic.

10. The method of claim 8, wherein the at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and any combination thereof.

11. The method of claim 1, wherein the cell or cell population comprises one or more cells from a bodily fluid, bodily excretion, a bodily secretion, muscle, liver, kidney, lung, heart, brain, intestine, stomach, pancreas, bladder, skin, or a combination thereof.

12. The method of claim 1, wherein the cell or cell population comprises one or more circulating mononuclear cell(s) and wherein the cell signature comprises a circulating mononuclear cell signature.

13. The method of claim 12, wherein the one or more circulating mononuclear cells comprise one or more peripheral blood mononuclear cells.

14. The method of claim 12, wherein the one or more circulating mononuclear cells comprise lymphocyte(s), monocyte(s), dendritic cell(s) or any combination thereof.

15. The method of claim 12, wherein the one or more circulating mononuclear cells comprise T cell(s), B cell(s), natural killer cell(s) or any combination thereof.

16. The method of claim 1, wherein the sample is a bodily fluid, a bodily excretion, a bodily secretion, a tissue, a cell or cell population, or a combination thereof.

17. The method of claim 16, wherein the sample is blood.

18. A method of diagnosing, prognosing, and/or monitoring a mitochondrial disease comprising:
detecting mitochondrial DNA (mtDNA) heteroplasmy and cell type, cell state, or both in a cell or cell population, wherein detecting comprises
detecting, in a sample comprising the cell or cell population, a cell signature in the cell or cell population, and
detecting mtDNA heteroplasmy in the cell or cell population,
wherein the cell signature and/or mtDNA heteroplasmy indicates at least cell type, cell state, or both; and
optionally repeating detecting mtDNA heteroplasmy and cell type, cell state, or both one or more times over a period of time.

19. The method of claim 18, wherein the cell signature comprises a chromatin accessibility signature, a gene expression signature, a protein expression signature, an epigenetic state signature, a cell surface marker expression signature, a cell activity signature, a phenotypic profile, a cell landscape, or a combination thereof.

20. The method of claim 18, wherein detecting the signature and/or detecting mtDNA heteroplasmy is/are determined by a sequencing method.

21. The method of claim 20, wherein the sequencing method comprises single cell RNA sequencing and/or mitochondrial DNA single cell ATAC-seq (mtscATAC-seq).

22. The method of claim 18, wherein detecting a cell signature comprises measuring a change in a distance in gene expression or accessible fragment space between two or more cell states.

23. The method of claim 22, wherein the gene expression and/or accessible fragment space comprises, 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

24. The method of claim 22, where the distance in gene expression and/or accessible fragment space is measured by a Euclidean distance, Pearson coefficient, Spearman coefficient, or combination thereof.

25. The method of claim 18, wherein detecting mtDNA heteroplasmy comprises detecting one or more mutations the mtDNA.

26. The method of claim 25, wherein at least one of the one or more mutations are pathogenic.

27. The method of claim 25, wherein the at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and any combination thereof.

28. The method of claim 18, wherein the cell or cell population comprises one or more cells from a bodily fluid, bodily excretion, a bodily secretion, muscle, liver, kidney, lung, heart, brain, intestine, stomach, pancreas, bladder, skin, or a combination thereof.

29. The method of claim 18, wherein the cell or cell population comprises one or more circulating mononuclear cell(s) and the cell signature comprises a circulating mononuclear cell signature.

30. The method of claim 29, wherein the one or more circulating mononuclear cells comprise one or more peripheral blood mononuclear cells.

31. The method of claim 29, wherein the one or more circulating mononuclear cells comprise lymphocyte(s), monocyte(s), dendritic cell(s) or any combination thereof.

32. The method of claim 29, wherein the one or more circulating mononuclear cells comprise T cell(s), B cell(s), natural killer cell(s) or any combination thereof.

33. The method of claim 18, wherein the sample is a bodily fluid, a bodily excretion, a bodily secretion, a tissue, a cell or cells, or a combination thereof.

34. The method of claim 33, wherein the sample is blood.

35. The method of claim 18, wherein the mitochondrial disease is a maternally inherited mitochondrial disease.

36. The method of claim 18, wherein the mitochondrial disease is a heteroplasmic mitochondrial disease.

37. The method of claim 18, wherein the mitochondrial disease is MELAS (mitochondrial myopathy encephalopathy, and lactic acidosis and stroke-like episodes), CPEO/PEO (chronic progressive external ophthalmoplegia syndrome/progressive external opthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease as set forth in any one or more of Tables 1-5, or any combination thereof.

38. A method of treating and/or preventing a mitochondrial disease or a symptom thereof in a subject in need thereof comprising:
diagnosing, prognosing, and/or monitoring a mitochondrial disease or a symptom thereof in the subject in need thereof as in any of claims 18-37, wherein the sample is from the subject in need thereof, and;
administering one or more agent(s) or formulations thereof to the subject in need thereof effective to treat and/or prevent the mitochondrial disease or symptom thereof.

39. A kit for diagnosing, prognosing, and/or monitoring a mitochondrial disease and/or determining segregation dynamics of mitochondrial DNA (mtDNA) comprising:
a collection vessel configured to collect and/or contain a sample comprising a cell or cell population obtained from a body of a subject, wherein the sample is a bodily fluid, a bodily excretion, a bodily secretion, a tissue, a cell or cell population, or a combination thereof;
instructions fixed in a tangible medium of expression that provides direction to collect the sample in the collection vessel and determine
a) segregation dynamics of mtDNA,
b) a diagnosis of a mitochondrial disease,
c) a prognosis of a mitochondrial disease, or
d) a combination thereof,
and optionally monitor any one or more of a)-d) by a method comprising:
detecting mitochondrial DNA (mtDNA) heteroplasmy and cell type, cell state, or both in the cell or cell population, wherein detecting comprises
detecting cell signature in the cell or cell population, and
detecting mtDNA heteroplasmy in the cell or cell population,
wherein the cell signature and/or mtDNA heteroplasmy indicates at least cell type, cell state, or both; and
optionally repeating detecting mtDNA heteroplasmy and cell type, cell state, or both in the cell or cell population one or more times over a period of time.

40. The kit of claim 39, wherein the cell signature comprises a chromatin accessibility signature, gene expression signature, protein expression signature, epigenetic state signature, a cell surface marker expression signature, a cell activity signature, a phenotypic profile, a cell landscape, or a combination thereof.

41. The kit of claim 39, wherein detecting the cell signature and/or detecting mtDNA heteroplasmy is/are determined by a single cell sequencing method.

42. The kit of claim 41, wherein the single cell sequencing method comprises single cell RNA sequencing and/or mitochondrial DNA single cell ATAC-seq (mtscATAC-seq).

43. The kit of claim 39, wherein detecting a cell signature comprises measuring a change in a distance in gene expression space and/or accessible fragment space between two or more cell states.

44. The kit of claim 43, wherein the gene expression and/or accessible fragment space comprises 1 or more genes and/or accessible fragments, 10 or more genes and/or accessible fragments, 20 or more genes and/or accessible fragments, 30 or more genes and/or accessible fragments, 40 or more genes and/or accessible fragments, 50 or more genes and/or accessible fragments, 100 or more genes and/or accessible fragments, 500 or more genes and/or accessible fragments, or 1000 or more genes and/or accessible fragments.

45. The kit of claim 43, where the distance in gene expression and/or accessible fragment space is measured by a Euclidean distance, Pearson coefficient, Spearman coefficient, or combination thereof.

46. The kit of claim 39, wherein detecting mtDNA heteroplasmy comprises detecting one or more mutations the mtDNA.

47. The kit of claim 46, wherein at least one of the one or more mutations are pathogenic.

48. The kit of claim 46, wherein the at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), a mutation as set forth in any one or more of Tables 1-5, and any combination thereof.

49. The kit of claim 39, wherein the cell or cell population comprises one or more cells from a bodily fluid, bodily excretion, a bodily secretion, muscle, liver, kidney, lung, heart, brain, intestine, stomach, pancreas, bladder, skin, or a combination thereof.

50. The kit of claim 39, wherein the cell or cell population comprises one or more circulating mononuclear cell(s) and the cell signature is a circulating mononuclear cell signature.

51. The kit of claim 50, wherein the one or more circulating mononuclear cells comprise one or more peripheral blood mononuclear cells.

52. The kit of claim 50, wherein the one or more circulating mononuclear cells comprise lymphocyte(s), monocyte(s), dendritic cell(s) or a combination thereof.

53. The kit of claim 50, wherein the one or more circulating mononuclear cells comprise T cell(s), B cell(s), natural killer cell(s) or a combination thereof.

54. The kit of claim 39, wherein the sample is a bodily fluid, a bodily excretion, a bodily secretion, a tissue, a cell or cells, or a combination thereof.

55. The kit of claim 54, wherein the sample is blood.

56. The kit of claim 39, wherein the mitochondrial disease is a maternally inherited mitochondrial disease.

57. The kit of claim 39, wherein the mitochondrial disease is a heteroplasmic mitochondrial disease.

58. The kit of any one of claims 39-57, wherein the mitochondrial disease is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), a cardiomyopathy, an encephalomyopathy, Pearson's syndrome, a disease as set forth in any one or more of Tables 1-5, or any combination thereof.

59. The kit of claim 39, wherein the collection vessel comprises a reagent effective to prepare and/or preserve the sample.

60. The kit of claim 39, wherein the collection vessel comprises a reagent effective to prepare and/or preserve the sample for detecting the cell signature and/or mtDNA heteroplasmy.

61. The kit of claim 39, wherein the collection vessel is physically and/or chemically configured to preserve and/or prepare the sample for detecting the circulating mononuclear cell signature and/or mtDNA heteroplasmy.
